(19) United States
(12) Patent Application Publication
(10) Pub. No.: US 2020/0165675 A1
(43) Pub. Date: May 28, 2020

(54) IDENTIFICATION OF EPIGENETIC SIGNATURES INDICATING CEREBRAL PALSY

(71) Applicants: Genome Profiling, LLC, Wayne, PA (US); The Nemours Foundation, Jacksonville, FL (US)

(72) Inventors: Adam G. Marsh, Lewes, DE (US); Robert E. Akins, JR., West Chester, PA (US); Erin L. Crowgey, Lincoln University, PA (US); Karyn G. Robinson, Wilmington, DE (US)

(73) Assignees: Genome Profiling, LLC, Wayne, PA (US); The Nemours Foundation, Jacksonville, FL (US)

(21) Appl. No.: 16/616,640

(22) PCT Filed: May 31, 2018
(86) PCT No.: PCT/US2018/035335

§ 371 Date: Nov. 25, 2019

Related U.S. Application Data
(60) Provisional application No. 62/603,486, filed on Jun. 1, 2017.

(51) Int. Cl. C12Q 1/6883

(52) U.S. Cl. CPC C12Q 1/6883; C12Q 2600/154

(57) ABSTRACT

In one aspect, the invention provides a method of detecting cerebral palsy in a subject. The method includes: determining a methylation state for each of a plurality of cytosine-guanine dinucleotide (CpG) sites in a sample obtained from the subject, calculating a differential methylation level based on the methylation states of the plurality of CpG sites, and comparing the differential methylation level to a predetermined reference level. When the differential methylation level deviates from the predetermined reference level, the presence of cerebral palsy is indicated in the patient.

### CROSS REFERENCE TO RELATED APPLICATIONS

The application claims priority to U.S. Provisional Patent Application No. 62/603,486, filed Jun. 1, 2017, which is incorporated herein by reference in its entirety.

### BACKGROUND OF THE INVENTION

Cerebral Palsy (CP) refers to a group of movement disorders with symptoms including poor coordination, stiff or weak muscles and tremors. There is currently no biomarker-based test to confirm or rule out CP or sub-classifications of CP.

### SUMMARY OF THE INVENTION

In one aspect, the invention provides a method of detecting cerebral palsy in a subject. The method includes: determining a methylation state for each of a plurality of cytosine-guanine dinucleotide (CpG) sites in a sample obtained from the subject, calculating a differential methylation level based on the methylation states of the plurality of CpG sites, and comparing the differential methylation level to a predetermined reference level. When the differential methylation level deviates from the predetermined reference level, the presence of cerebral palsy is indicated in the patient.

In various embodiments, the plurality of CpG sites comprises at least one selected from the CpG sites listed in Table 4, Table 5, Table 6 or Table 7.

In various embodiments, the plurality of CpG sites comprises at least five selected from the CpG sites listed in Table 4, Table 5, Table 6 or Table 7.

In various embodiments, the plurality of CpG sites comprises at least ten selected from the CpG sites listed in Table 4, Table 5, Table 6 or Table 7.

In various embodiments, the plurality of CpG sites comprises at least m % selected from the top n most predictive CpG sites listed in Table 4, Table 5, Table 6 or Table 7. wherein: m is selected from the group consisting of: 50, 60, 70, 80, 90, 95, and 99; and n is selected from the group consisting of: 100, 500, 1,000, 2,000, and 2,500.

In various embodiments, the method further includes providing treatment for cerebral palsy to the patient when cerebral palsy is indicated. In various embodiments, the treatment for cerebral palsy includes the administration of medication, therapy or surgery.

In various embodiments, the plurality of CpG sites includes a plurality of up sites and a plurality of down sites. Methylation at the up sites indicates that the likelihood the patient has cerebral palsy is increased. Methylation at the down sites indicates that the likelihood the patient has cerebral palsy is decreased. Calculating a differential methylation level includes adding in a linear weighted summation values based on the methylation states of the up sites from a value based on the methylation states of the down sites.

In various embodiments, the sample is selected from the group consisting of a peripheral blood mononuclear cell sample, a blood sample, a muscle sample or a satellite cell sample. The blood sample may either be a whole blood sample or a buffy coat sample.

### BRIEF DESCRIPTION OF THE DRAWINGS

For a fuller understanding of the nature and desired objects of the present invention, reference is made to the following detailed description taken in conjunction with the accompanying figures.

FIG. 1 depicts sample profiles for % MET as a frequency distribution (histogram) overlaid with a probability density distribution function (filled logistic curve). The data reveal bulk profiles of methylation that were scored in the analysis described herein.

FIG. 2 is a series of graphs which depict a high-level breakdown of methylation load (ΔML) among functional class and gene structure categories to identify most active regions of methylation changes between non-CP vs CP. Differential methylation load is used as a scoring metric for considering how total methylation intensity for a given gene or gene region differs between sample groups.

FIG. 3 depicts epigenetic discrimination of methylation patterns. Using an ordinate analysis technique of non-metric multidimensional scaling (NMDS), CpG methylation profiles were compared among individuals to isolate patterns conserved within groups while also differing between groups (non-CP vs CP). The first two component axes are plotted to locate the individual sample points in a 2D plane. Ellipses drawn represent 90% confidence intervals around the position of the true group means.

FIG. 4 depicts the frequency distribution of the coefficient of variation for each CpG(i) site among sample replicates within both non-CP and CP cohorts plotted for each pairwise comparison. This reveals the level of variance among replicate measures in the analysis. A 10% reference line is shown. The inset shows the P-value significance level of the top 5,000 CpG sites scored in the pairwise analysis. Sample comparisons with low variance have highly significant differences.

FIG. 5 depicts the top 1,000 differentially methylated CpG sites in each pairwise comparison clustered with a visual heatmap of % Met scores (yellow=0% to blue=100%). These individual CpG sites are then tested for significance with a false-discovery rate correction. The resolution between non-CP and CP samples reveals quantitative separation among distinct subgroups of CpG sites.

FIG. 6 is a bar graph that depicts the P-value of groups of CpG sites by P-value cutoff.

FIG. 7 depicts a hierarchical clustering of methylation patterns among samples to identify those sites or domains with correlated shifts in methylation.

FIG. 8 is a smear plot which depicts CpG response distribution from likelihood ratio test in FIG. 7. NS=Not Significant; PVAL=P-value; FDR=False Discovery Rate.

FIG. 9 is a volcano plot that depicts CpG fold-change vs. P-value. NS=Not Significant; PVAL=P-value; FDR=False Discovery Rate.

FIG. 10 depicts differential methylation load with top 10 CpG Sites. Circular ideograms are presented with a mean subtraction of CpG methylation scores (Grp1 minus Grp2) to calculate a summation methylation load (ΔML) score across chromosomal domains. These data are presented as a scatterplot with red and green background to accentuate areas where they are most different. The correlative association between top 10 CpG sites are shown as red arcs that track the first highest correlation for each CpG site while orange arcs show the second highest. Gene labels indicate the positions of the top 60 CpG sites, with green indicating higher methylation in Grp1 and red indicating higher methylation in Grp2.

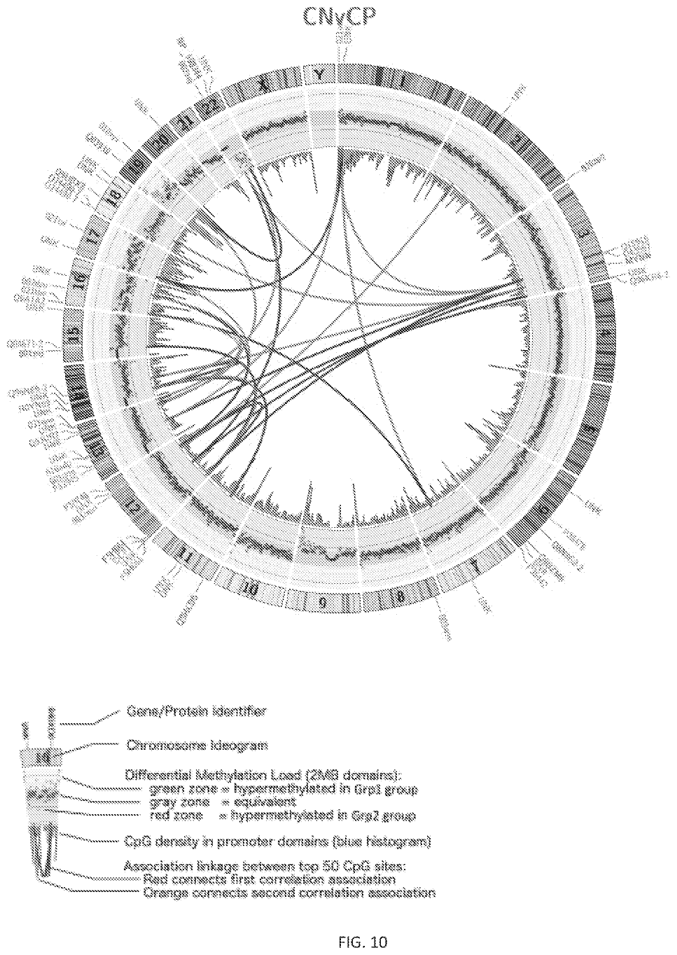

FIG. 11 contrasts overall CpG site methylation between CP and non-CP subjects. Significantly different (sigdiff) differential methylation was defined as p<0.05, was observed at 4,694 sites in muscle cells. Overall, CpG sites in CP muscle samples are more unmethylated than in Controls.

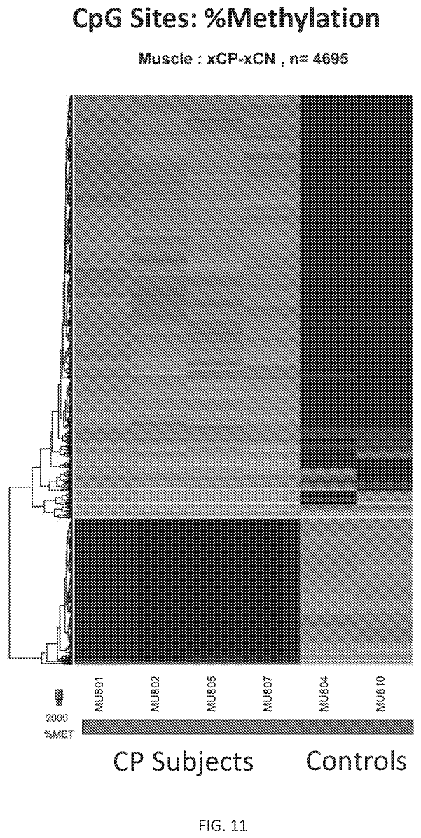

FIG. 12 depicts transcriptional start site (TSS) methylation load score in muscle cell samples. There are significantly different differential methylation load scores between CP and non-CP subjects based on CpG sites in the promoter regions of 120 annotated genes. There is no prominent overall methylation loss or gain between CP and Control in TSS domains.

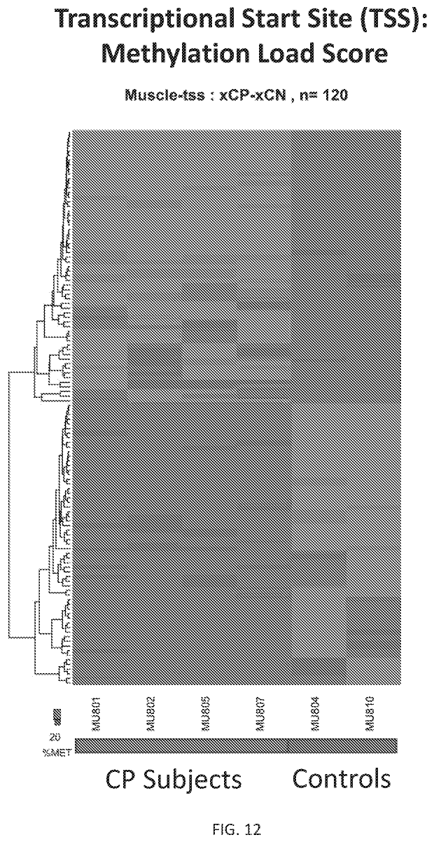

FIG. 13 contrasts overall CpG site methylation between CP and non-CP subjects. SigDiff differential methylation (p<0.05, FDR) at 4,694 sites in myosatellite cells (SAT cells). Overall CpG sites in CP SAT cell samples are more slightly more unmethylated than in Controls.

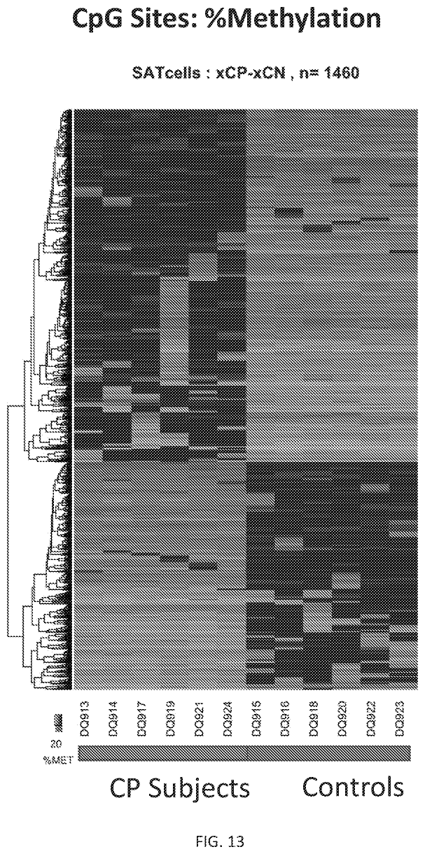

FIG. 14 depicts transcriptional start site (TSS) methylation load score in SAT cell samples. There are significantly different differential methylation load scores between CP and non-CP subjects based on CpG sites in the promoter regions of 36 annotated genes. There is no prominent overall methylation loss or gain between CP and Control in TSS domains.

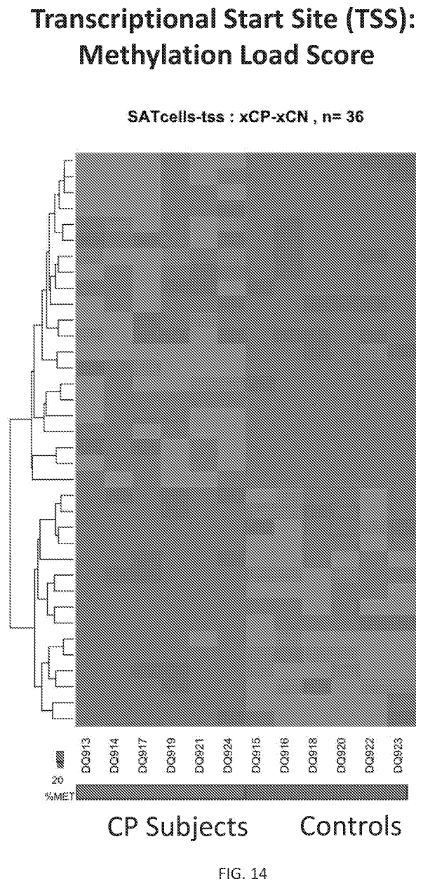

FIG. 15 contrasts CpG site % methylation between CPwb (Cp, whole blood samples), CNwb (non-CP, whole blood samples), CPbc (CP, buffy coat samples) and CNbc (non-CP, buffy coat samples).

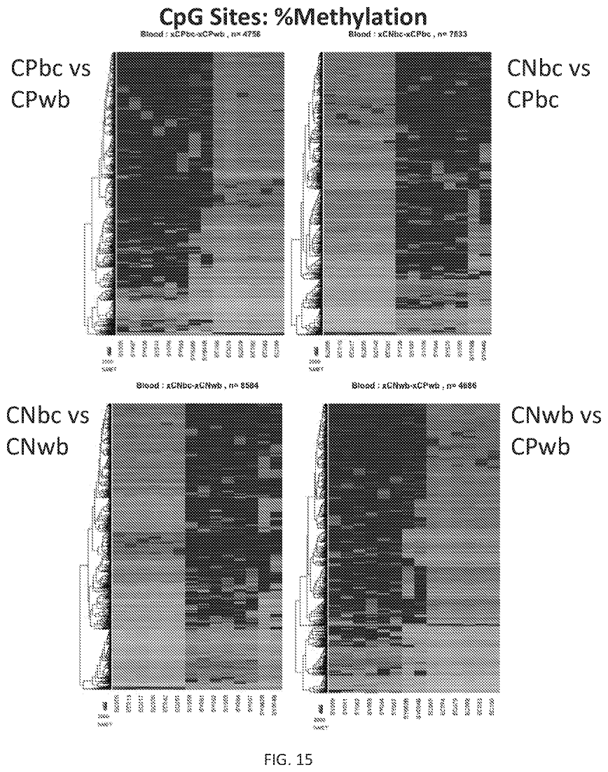

FIG. 16 depicts the number of CpG sites that are significantly different between each sample type. The highlighted slice represents the 585 CpG sites that are SigDiff between CP and CN in both whole blood and buffy coat.

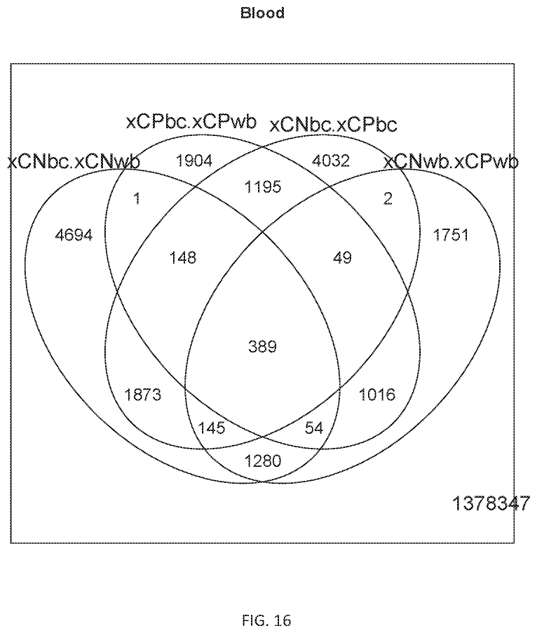

FIG. 17 depicts transcriptional start site (TSS) methylation load score in CPwb (Cp, whole blood samples), CNwb (non-CP, whole blood samples), CPbc (CP, buffy coat samples) and CNbc (non-CP, buffy coat samples). There are 59 unique sigdiff TSS sites in CP vs CN for whole blood. There are 109 unique sigdiff CpG sites in Cp vs CN for buffy coat. The yield of sigdiff sites is higher in buffy coat, but the discrimination between patient phenotypes is equivalent between WB and BC.

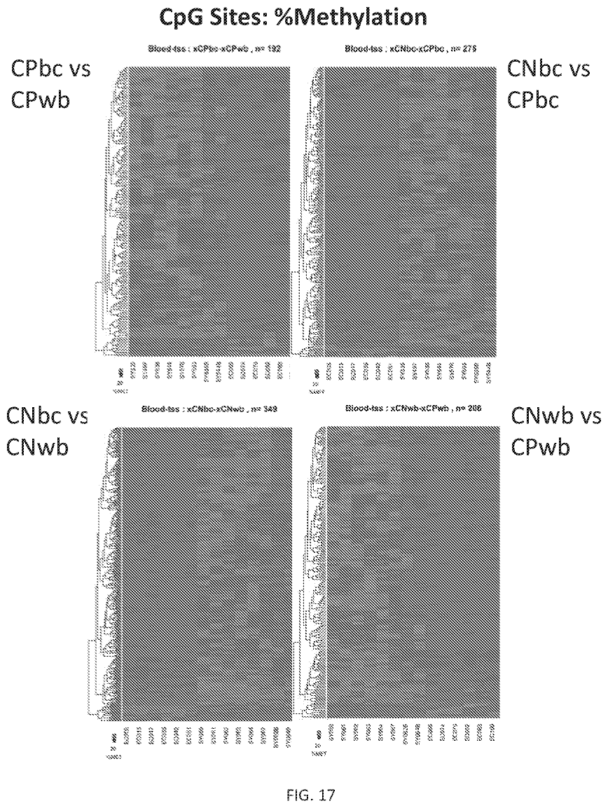

FIG. 18 depicts the number of TSS Gene sites that are significantly different between each sample type. The highlighted slice represents the 36 TSS Gene sites that are significantly different between CP and CN in both whole blood and buffy coat.

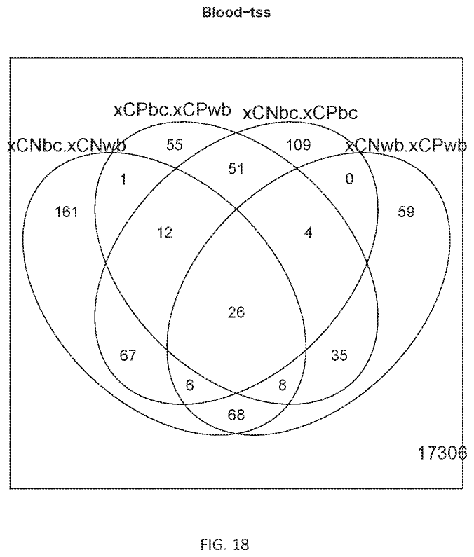

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

As used herein, “cerebral palsy” or “CP” refers to a superfamily of disorders generally considered to be caused by damage to or abnormalities within the developing brain and associated with impaired motor function including movement, posture, and balance.

As used herein, “cytosine-guanine dinucleotide site” or “CpG site” means a cytosine nucleotide followed by a guanine nucleotide in the genome of an organism. CpG sites can be designated with the number of the chromosome of the organism on which they are located and a number designating the position. The flanking sequences can be used to generate the position number. For example, “12.108079458” or “chr12 108079458” refer to a CpG site on chromosome 12 at position 108079458. The position number refers to the nucleotide index starting from 1 on the coding or plus (+) strand of the DNA molecule and specifically references the position of the 5′ cytosine in a CpG dinucleotide pair. In addition, this CpG location has a complementary sequence pair on the non-coding minus (−) strand and the position number also refers to that complementary strand cytosine which is located plus one nucleotide from the indicated coding strand position. Thus, for CpG 12.108079458, the methylation score values indexed to this specific site cover the cytosine on the coding strand of chromosome 12 at position number 108079458 and the cytosine on the non-coding strand of chromosome 12 at position number 108079459.

As used herein, “methylation” or “methylated” as applied to CpG sites refers to the addition of a methyl group to cytosine, forming either 5′-methyl-cytosine or 5′-hydroxymethyl-cystosine.

As used herein, the term “percent methylation” or “% MET” refers to the frequency with which a particular set of CpG sites are methylated. Here, CpG methylation is expressed as a percentage of methylated copies found in the DNA sample for each individual CpG site relative to the total number of copies found for each site.

A “reference level” with respect to some measurement used in diagnosis is indicative of the presence or absence of a particular phenotype or characteristic. When the level of the measurement in a subject deviates from the reference level it is indicative of the presence of, or relatively heightened level of, a particular phenotype or characteristic.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Conclusive diagnosis of CP is valuable because it is a prerequisite for intervention and affords peace of mind to the patient and family. Accordingly, there is a need for an efficient and accurate test for CP. This disclosure addresses that need.

One aspect of the invention provides a method of detecting cerebral palsy in a subject. The method can include: determining a methylation state for each of a plurality of CpG sites in a sample obtained from the subject, calculating a differential methylation level based on the methylation states of the plurality of CpG sites, and comparing the differential methylation level to a predetermined reference level. Deviation of the methylation load level from the predetermined reference level indicates the presence of cerebral palsy in the patient.

The methylation state of the plurality of CpG sites may be determined by any means known in the art. In various embodiments, the methylation state of the plurality of CpG sites may be determined by methyl-sensitive restriction enzyme digestion followed by Next-Gen Sequencing on an appropriate instrument, or they may be determined by targeted qPCR assays to quantify cut and uncut CpG sites following methyl-sensitive restriction enzyme digestion, or they may be determined by bisulfite oxidation treatment with DNA sequencing (either direct or via NGS), or they may be determined by hybridization of labelled oligonucleotide probes (called “hybridization arrays”) to measure methylation following methyl-sensitive restriction enzyme digestion, or they may be determined by hybridization of anti-5′-methyl-cytosine antibodies to measure methylation after hybridization capture on a targeted gene panel.

Without wishing to be limited by theory, in various embodiments, the method relies on the concept of differential methylation level (ΔML)—site-specific differences in CpG methylation summed across a gene or genome domain, structure or element—in order to characterize functional shifts in methylation patterns. This method is illustrated in the example below and in Equation (1).

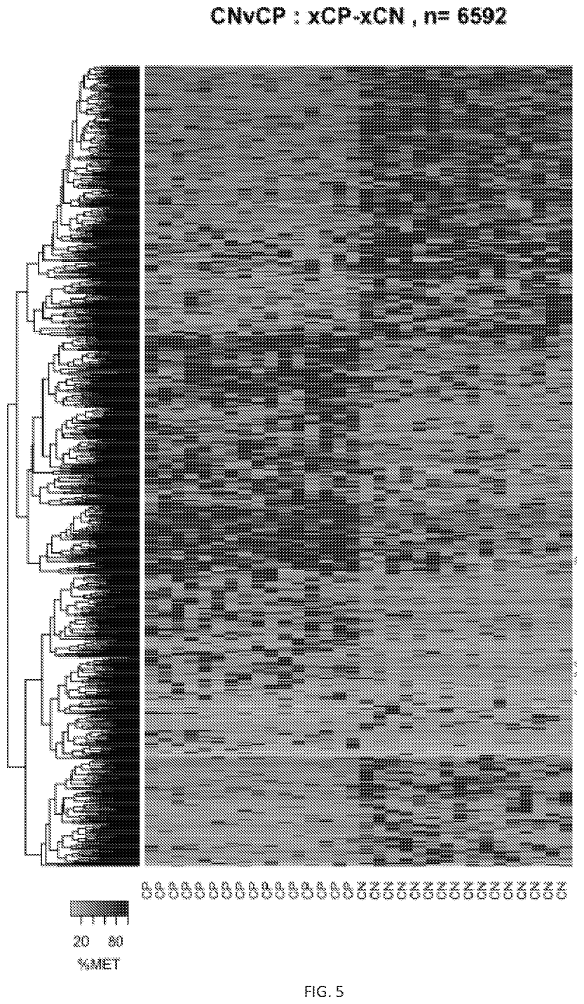

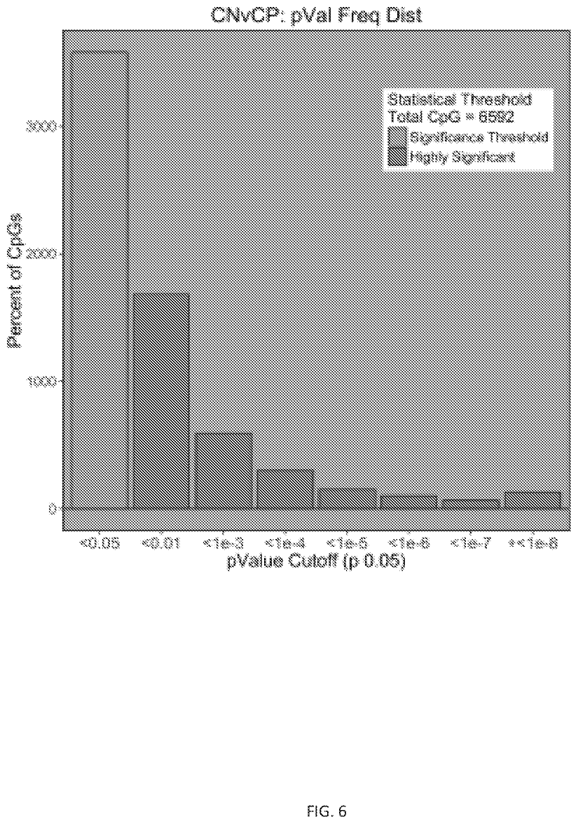

As shown in FIGS. 5 and 6, the methylation state of the majority of CpG sites in the genome are not significant predictors of CP in a patient. However, determining the methylation state of a certain subset of CpG sites and applying the algorithm outlined above or a derivative classification algorithm one may be used to detect CP. The plurality of CpG sites in the present aspect of the invention refers to sites selected from this predictive subset of sites. In various embodiments, the sites may be selected from Table 4, Table 5, Table 6 and Table 7.

In various embodiments, the plurality of CpG sites includes a plurality of “up sites” and a plurality of “down sites”. Up sites are CpG sites where methylation at the site indicates an increased methylation load in CP patients relative to non-CP matched control patients. Down sites are CpG sites where methylation at the site indicates a decreased methylation load in CP patients relative to non-CP matched control patients. Table 3 illustrates one possible approach by which these sites may be scored in order to determine ΔML.

Various embodiments of the invention are directed to methods of examining sets of up sites and down sites and determining a differential methylation level based on their methylation state. Due to the predictive value of the plurality of sites, the scores will differ when examining patients with and without CP.

A predetermined reference level may be calculated by determining the methylation state of a set of sites in patients who are known not to have CP. Accordingly, when a differential methylation level is determined using the same plurality of site, deviation from the reference level indicates an additional evidentiary datum point supporting increasing the probability that the patient likely has CP.

A skilled person will understand that the specifics of the calculation used for generating a differential methylation level are not critical and various processes may be employed to generate these levels. All of them are within the scope of various embodiments of the invention.

Various embodiments of the invention rely on pluralities of CpG sites of various sizes. In various embodiments, the plurality of CpG sites may contain about 5, 10, 100, 1000, 5000 or more CpG sites.

Even among the CpG sites that have shown predictive power, different sites contribute different weightings to the overall predictive probability of the CP status of a patient. Various embodiments of the invention calculate differential methylation levels based on various combinations of predictive CpG sites.

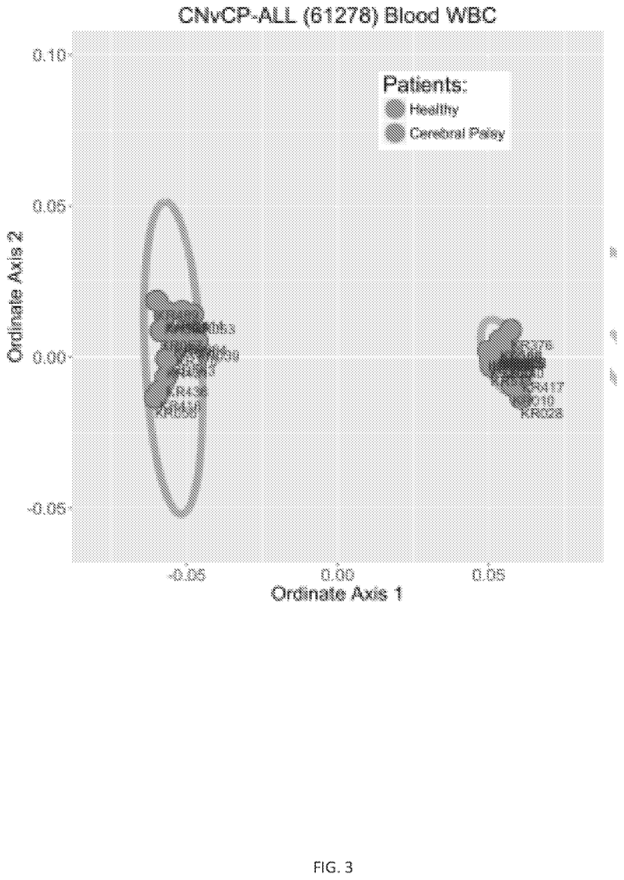

The predictive power of the sites may be quantified in various ways. As shown in FIG. 3, the deviation between the reference level (non-CP) and the differential methylation level for patients with CP can be represented by non-metric multidimensional scaling (NMDS). Each site in the set will make a contribution to the ordinate discrimination in the NMDS. In some embodiments, the plurality of CpG sites includes one or more sites selected from Table 4, ranked by the site's contribution to ordinate discrimination. Table 6 lists the top 1000 CpG sites for blood, muscle and muscle satellite cell samples by P-value.

The predictive power of CpG sites may also be quantified based on P-value, adjusted for False Discovery Rate (FDR). Table 5 lists the top 40 CpG sites for peripheral blood mononuclear cell samples by P-value. In various embodiments, the plurality of CpG sites may include one or more sites selected from Table 5. In some embodiment, the plurality of CpG sites may include the top n sites or m of the top n sites from Table 5 or other ordinal lists (wherein m and n are positive integers). Table 7 lists the top 1000 CpG sites for blood, muscle and muscle satellite cell samples by P-value.

In various embodiments, the method further includes providing treatment for CP to patients in whom CP is indicated. The treatment may include any form of standard of care treatment for CP accepted by the extended medical community. CP as a group of disorders includes many variations that require various interventions to improve the quality of life of the CP patient and any of these may be included in various embodiments of the invention.

The method may be practiced by analyzing a variety of sample types. In various embodiments, the sample may be a peripheral blood mononuclear cell sample, a blood sample, a muscle cell sample or muscle satellite cell sample. In various embodiments, the sites selected from Table 4, Table 5, Table 6 or Table 7 can be matched to the sample type used.

In various embodiments the treatment for CP may include surgery. In various embodiments, the surgery may include gastroenterology surgery, hearing correction surgery, medicine related surgery, e.g. insertion of a baclofen pump, orthopedic surgery, neurosurgery, urologic surgery and vision correction surgery.

In various embodiments the treatment for CP may be therapy. In various embodiments the therapy may include occupational therapy, physical therapy and speech therapy.

In various embodiments, the treatment for CP may include the administration of medication. In various embodiments the administration of medication may include medication to treat seizures, involuntary movement, spasticity, incontinence and gastroesophageal reflux. In various embodiments the treatment for CP may include anticonvulsants, muscle relaxants, benzodiazepines, nerve blocks, botulinum toxin (BTX) (available, e.g., under the BOTOX® trademark from Allergan, Inc. of Irvine, Calif.), baclofen and anticholinergics.

### Experimental Example

The invention is further described in detail by reference to the following experimental example. This example is provided for purposes of illustration only, and is not intended to be limiting unless otherwise specified. Thus, the invention should in no way be construed as being limited to the following example, but rather, should be construed to encompass any and all variations which become evident as a result of the teaching provided herein.

### Example 1

A cohort of 34 patients was identified and genomic DNA was extracted from each patient. Samples were prepared for sequencing analysis using a methyl-sensitive restriction enzyme digestion and library preparation followed by Next-Gen Sequencing (NGS) on an ILLUMINA® HISEQ® X10™ system with >30× coverage. Sequencing data was uploaded to secure cloud-based computational infrastructure where it was processed and analyzed for site-specific DNA methylation profiles.

The samples used in this study are listed in Table 1 with the codes employed for analyses and the genomic DNA QC values. The sequencing vendor Macrogen, Inc. performed a second round of QC upon sample receipt. Genomic DNA samples received from Nemours Biomedical Research Center were coded internally with GP numbers. DNA quantity and quality used for sequencing is reported in the data columns.

TABLE 1
Sample Identification Codes.

| Blinding | | | | | |
|---|---|---|---|---|---|
| Code | ng/uL | A260 | 260/230 | 260/280 | Total ug |
| 10 | 518.57 | 10.371 | 2.35 | 1.88 | 10.3714 |
| 28 | 2867.9 | 57.36 | 2.32 | 1.87 | 57.358 |
| 370 | 982.95 | 19.659 | 2.3 | 1.91 | 19.659 |
| 375 | 1327.1 | 26.542 | 2.34 | 1.92 | 26.542 |
| 376 | 263.82 | 5.276 | 2.35 | 1.91 | 5.2764 |
| 380 | 1049.7 | 20.995 | 2.08 | 1.76 | 20.994 |
| 414 | 264.88 | 5.298 | 2.18 | 1.91 | 5.2976 |
| 416 | 1447.9 | 28.959 | 2.31 | 1.91 | 28.958 |
| 417 | 510.25 | 10.205 | 2.29 | 1.88 | 10.205 |
| 425 | 664.05 | 13.281 | 2.28 | 1.85 | 13.281 |
| 431 | 1216.6 | 24.334 | 2.33 | 1.92 | 24.332 |
| 436 | 1596.8 | 31.937 | 2.31 | 1.91 | 31.936 |
| 440 | 4258 | 85.7 | 2.09 | 1.7 | 85.16 |
| 444 | 418.42 | 8.368 | 2.38 | 1.89 | 8.3684 |
| 466 | 517.38 | 10.348 | 1.65 | 1.58 | 10.3476 |
| 489 | 543.51 | 10.87 | 2.3 | 1.88 | 10.8702 |
| 515 | 1724.4 | 34.49 | 2.34 | 1.91 | 34.488 |
| 527 | 1611.8 | 32.236 | 2.32 | 1.9 | 32.236 |
| 563 | 2060.7 | 41.216 | 2.32 | 1.9 | 41.214 |
| 573 | 1405 | 28.102 | 2.16 | 1.91 | 28.1 |
| 583 | 2301.6 | 46.033 | 2.28 | 1.89 | 46.032 |
| 584 | 1794.9 | 35.9 | 2.33 | 1.9 | 35.898 |
| 597 | 383.91 | 7.678 | 2.22 | 1.91 | 7.6782 |
| 605 | 3157.9 | 63.158 | 2.32 | 1.86 | 63.158 |
| 533 | 3132.20 | 62.644 | 2.25 | 1.86 | 62.6 |
| 568 | 2294.30 | 45.887 | 2.32 | 1.89 | 45.9 |
| 600 | 2377.20 | 47.545 | 2.37 | 1.85 | 47.5 |
| 606 | 135.75 | 2.715 | 3.01 | 1.93 | 2.7 |
| 45 | 1340.3 | 26.807 | 2.44 | 1.92 | 26.8 |
| 50 | 338.85 | 6.777 | 2.43 | 1.90 | 6.8 |
| 39 | 889.07 | 17.781 | 2.37 | 1.88 | 17.8 |
| 53 | 1367.70 | 27.355 | 2.41 | 1.88 | 27.4 |
| 56 | 3760.60 | 75.213 | 2.22 | 1.82 | 75.2 |
| 57 | 2528.10 | 50.563 | 2.30 | 1.88 | 50.6 |

Statistical analyses were conducted for pairwise contrasts using a code system of “xGrp1.xGrp2” where Grp1 and Grp2 designate the sample groups as: non-CP (CN) and Cerebral Palsy (CP). When differential methylation loads (ΔML) are presented, those values are calculated as the difference of Grp1 minus Grp2. Thus, if ΔML>0, methylation in Grp1 is greater than in Grp2 and vice versa. The specific contrasts with sample information are presented in Table 2 (below).

TABLE 2

| | | | Samples | | |
|---|---|---|---|---|---|
| Code | Grp1 | Grp2 | Group 1 - Group 2 | | |
| Pairwise | Non-CP | CP | KR039, KR053, KR056, KR057, | - | KR010, KR028, KR045, KR050, |
| Contrasts | | | KR370, KR380, KR414, KR416, | | KR375, KR376, KR417, KR440, |
| CNvCP | | | KR425, KR431, KR436, KR466, | | KR444, KR533, KR568, KR573, |
| | | | KR489, KR527, KR563, KR584 | | KR583, KR597, KR600, KR606 |

Note that the statistical results reported in this document utilized a total patient cohort size of 32 (16 CN vs 16 CP) after two samples were omitted due to young age of patients (<5 yo).

ΔML was calculated as the summation of the difference in % MET scores for each CpG site within the defined region or structure being scored, averaged by the number of CpG sites present:

$$Δ ML = {\sum }_{i = first}^{last} [ {CpG}_{[ i ] [ Grp 1 ]} - {CpG}_{[ i ] [ Grp 2 ]} ] \qquad ( 1 )$$

where first and last CpG indexes are defined by the gene unit across which the summation score is being calculated. As the calculation is applied in Example 1, Grp1 is a control (non-CP) cohort and Grp2 contains patients with CP. Thus, positive ΔML values indicate more methylation present in Grp1 and negative values indicate more methylation in Grp2.

In this study, results from 5 levels of analyses were compared: (a) single CpG sites, (b) CpG Islands, (c) micro-CpG Islands, (d) 2 kb upstream promoter domains, and (e) Transcriptional Start Site (TSS) domains. The single CpG sites are simply the comparison of the individual site methylation score values. The CpG Islands are defined by the UCSC Genome Browser bed file and contain domains with greater than 50% G+C nucleotide content, greater than 0.60 ratio of Observed to Expected frequency of CpG sites, and a minimum length of 200 nucleotides. A subset of smaller domain definitions was also employed. The TSS domains are demarcated as a 2 kb window centered on the TSS (+/−1 kb down/up-stream). The methylation signal across the larger domain structures were all non-informative. This means that a discriminating signal was only observed at the level of single CpG site scores. Only the CpG data is included here in this report.

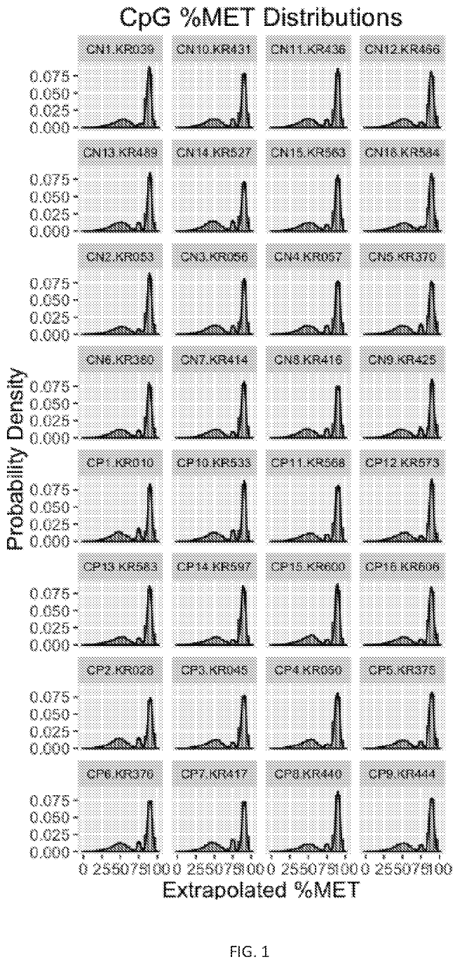

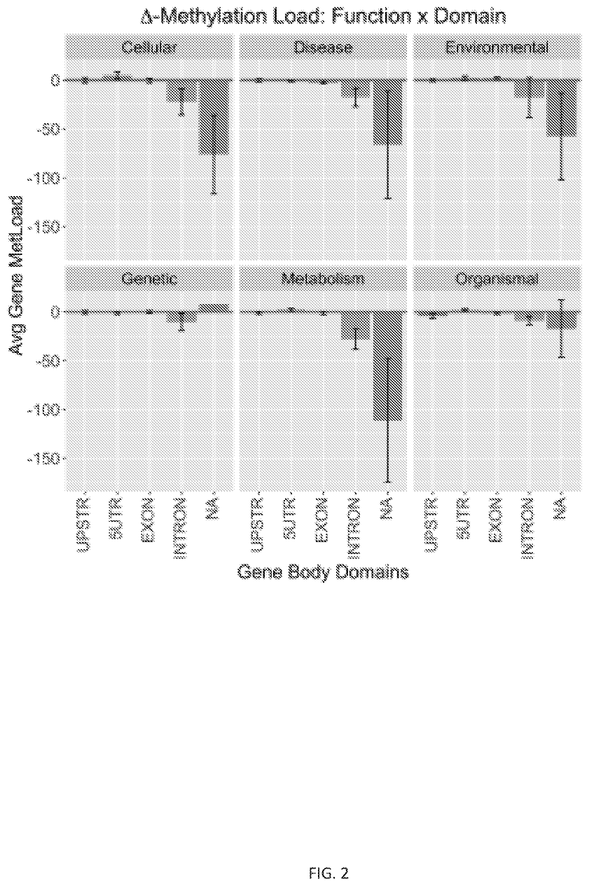

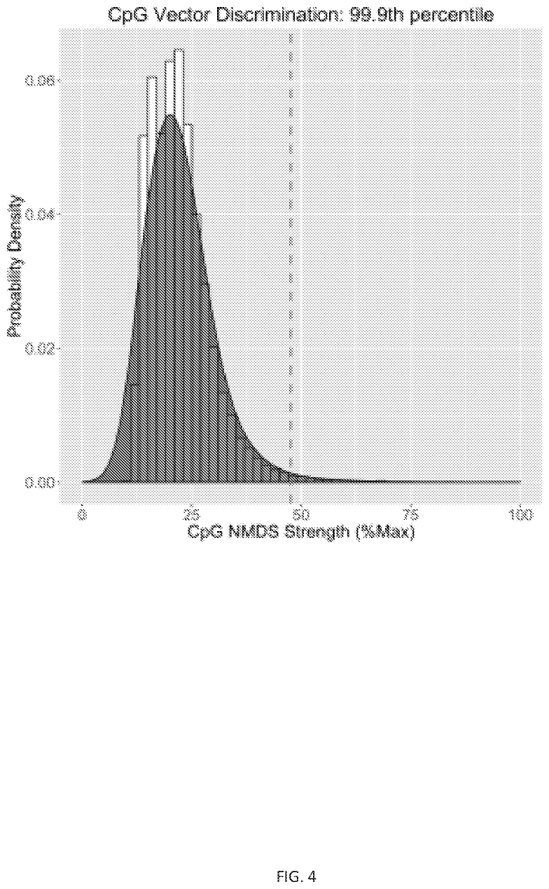

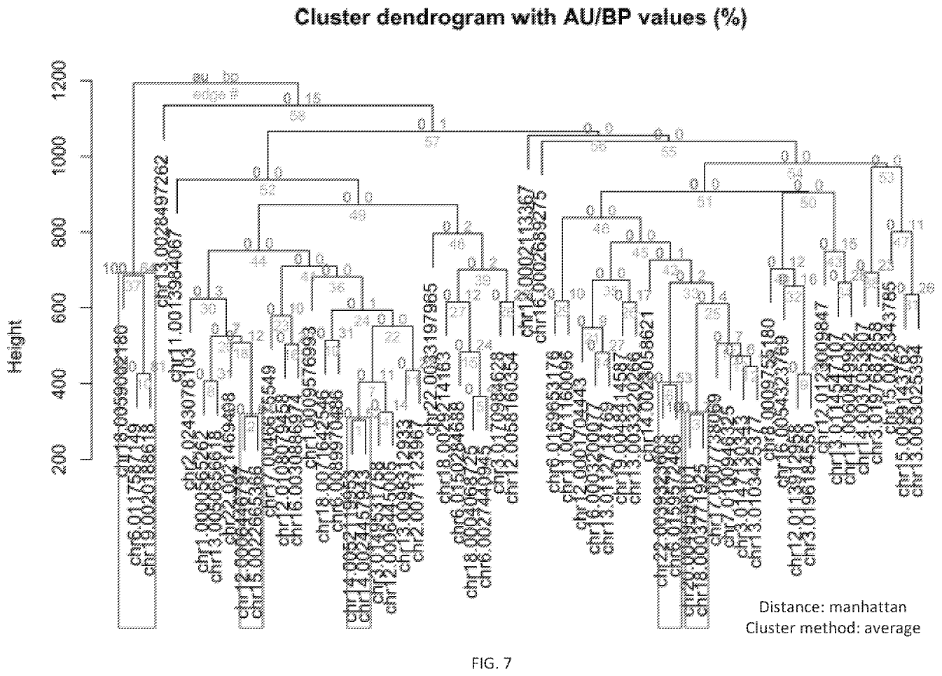

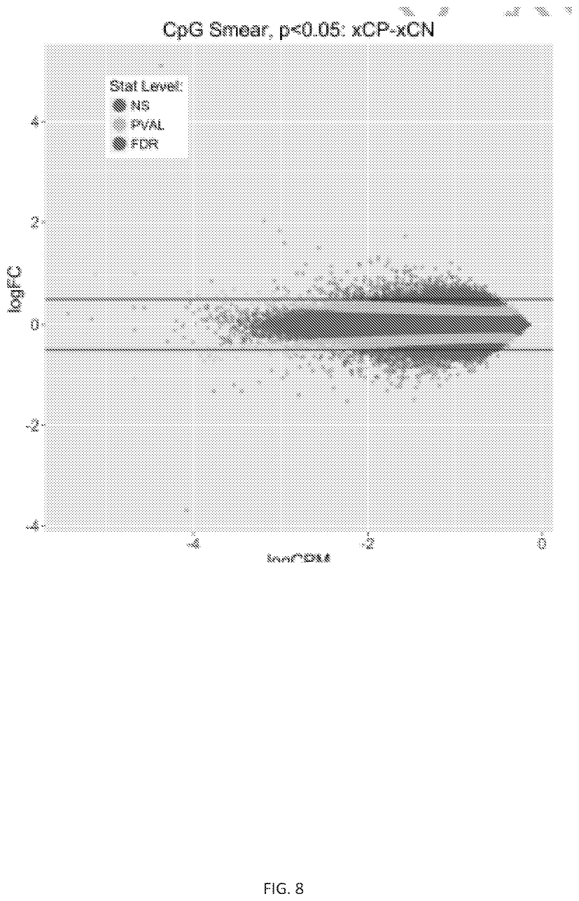

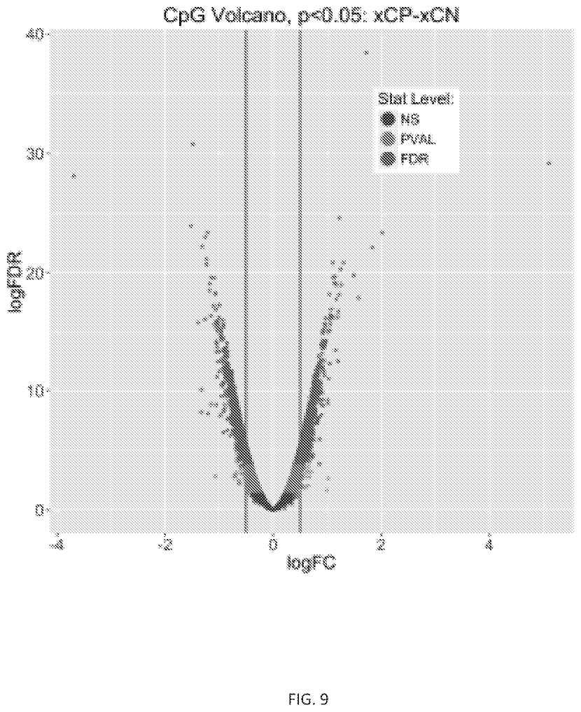

The pairwise analysis in Table 2 provides a direct contrast and allows for several graphical visualization plots to be generated these are presented in FIGS. 1-9. Statistically, the most robust analytical results are obtained from the multiway analysis, presented in FIG. 5. A Likelihood-Ratio-Test with one-way ANOVA-defined contrasts was used to establish a more conservative assessment than the pairwise comparisons because there is a better estimate of the false discovery rate (FDR) when the measurement variances for all samples are included as one total set. In all cases where P-values are reported in this document, significance is determined from this group multiway analysis after FDR correction.

Table 3 presents summation counts of the significant changes in methylation scores based on P-values adjusted for site-specific false discovery rates (FDR). The comparisons are executed as non-CP-minus-CP cohorts in the treatment code header, such that positive values mean methylation in the non-CP cohort is greater (UP) and negative values mean methylation in the CP cohort is greater (DOWN). Site-specific dispersion was estimated to equalize CpG variances. A Likelihood Ratio Test was used with a defined one-way ANOVA model for pairwise tests.

TABLE 3
Significant UpDown Changes in Methylation Scores.
CNvCP: CpG Sites

| Change | 0 | xCP · xCN |
|---|---|---|
| Down: −1 | NA | 2811 |
| Ns: 0 | NA | 1524163 |
| UP: +1 | NA | 3781 |

ns = “not significant”; Response = UP or DOWN in non-CP relative to CP cohort.

The following tables rank CpG sites according to various metrics associated with their predictive power or their location in various genes or pathways.

TABLE 4
Top 40 CpG Sites. Rank ordered listing of CpG sites contributing the most to the
ordinate discrimination in the NMDS analysis.

| CHR | POS | DOMAIN | UniProt | GO.terms |
|---|---|---|---|---|
| chr12 | 108079458 | upstr | B4DJV5 | transcription; protein.binding; |
| chr12 | 6446797 | intron | F5H6Z2 | positive.regulation.; modulation.by.virus.; prostaglandin.me |
| chr12 | 6445005 | intron | F5H6Z2 | positive.regulation.; modulation.by.virus.; prostaglandin.me |
| chr13 | 33220266 | intron | A9IYQ1 | binding; mitotic.sister.chrom; regulation.of.cell.p; mitotic |
| chr12 | 1704443 | intron | F5GYM2 | receptor.binding; negative, regulation.; Wnt.signaling.pathwa |
| chr6 | 169653176 | intron | Q5R153 | cell.adhesion; negative.regulation.; heparin.binding; calciu |
| chr18 | 4068725 | intron | A0A0A0MTP4 | cell-cell.signaling; synaptic.transmissio; protein.binding |
| chr12 | 58160354 | upstr | V9GYP0 | positive.regulation.; positive.regulation.; calcitriol .biosy |
| chr22 | 33197965 | na | A0A0A0MR68 | neurotransmitter.sec; catalytic.activity; ATP.binding; prote |
| chr11 | 71160096 | intron | E9PRL8 | 7-dehydrocholesterol; regulation.of.choles; sterol.biosynthe |
| chr3 | 197687858 | intron | F8WcE5 | cell.adhesion; mitosis; metal.ion.binding; proteolysis; meta |
| chr18 | 3771925 | intron | A0A0A0MTP4 | cell-cell.signaling; synaptic.transmissio; protein.binding |
| chr6 | 150284688 | upstr | Q9BZM6 | natural.killer.cell.; natural.killer.cell.; natural.killer.c |
| chr3 | 147130343 | exon | Q15915 | metal.ion.binding; regulation.of.smooth; positive.regulation |
| chr18 | 29214163 | intron | J3QQY9 | keratan.sulfate.meta; keratan.sulfate.bios; protein.N-Iinked |
| ch19 | 20188618 | upstr | Q03938 | regulation.of.transc; nucleic.acid.binding; DNA.binding; met |
| chr13 | 28497262 | intron | P52945 | response.to.vitamin; type.B.pancreatic.ce; detection.of.gluc |
| chr7 | 130353922 | exon | c9JVS7 | unk |
| chr6 | 7394523 | intron | E9PFQ8 | transferase.activity; protein.phosphorylat; protein.kinase.a |
| chr19 | 40723542 | exon | Q8N6N2 | protein.binding; biological.process; molecular.function |
| chr15 | 99344316 | intron | HOYLE7 | inactivation.of.MAPK; insulin-Iike.growth.; insulin.binding; |
| chr11 | 32112972 | exon | HOYDA4 | in.utero.embryonic.d; camera-type.eye.deve; calcium.ion.bind |
| chr11 | 188098 | intron | E9PQJ6 | protein.binding; SNAP.receptor.activi; retrograde.transport; |
| chr12 | 121082579 | intron | C9J8G2 | nuclear.localization; enzyme.inhibitor.act; negative.regulat |
| chr17 | 11145564 | intron | V9GZ25 | unk |
| chr20 | 5081567 | exon | Q5JWB9 | unk |
| chr3 | 182928999 | intron | Q9J326 | regulation.of.Rho.pr; Rho.guanyl-nucleotid; protein.binding |
| chr12 | 5019351 | 5utr | Q09470 | potassium.ion.transm; delayed.rectifier.po; potassium.channe |
| chr13 | 93896533 | intron | Q9Y625 | phototransduction; chondroitin.sulfate.; retinoid.metabolic. |
| chr11 | 32113404 | intron | H0YDA4 | in.utero.embryonic.d; camera-type.eye.deve; calcium.ion.bind |
| chr17 | 7975795 | upstr | 075342 | Iinoleate.9S-Iipoxyg; establishment.of.ski; arachidonate.12- |
| chr16 | 58000211 | intron | H3BQc3 | intracellular.cAMP.a; intracellular.cGMP.a; Iigand-gated.ion |
| chr18 | 57357804 | intron | A0A0A0MS88 | lymphangiogenesis; venous.blood.vessel.; collagen.binding; c |
| chr11 | 6633681 | upstr | Q12962 | histone.deubiquitina; histone.H3.acetylati; histone.acetyltr |
| chr6 | 26235050 | exon | P16402 | nucleosome.assembly; DNA.binding; protein.binding; nucleosom |
| chr12 | 69005196 | intron | F5H7Y6 | regulation.of.cell.j; Rap.protein.signal.t; energy.reserve.m |
| chr16 | 50582980 | intron | Q969G9 | negative.regulation.; positive.regulation.; regulation.of.ce |
| chr12 | 4758511 | intron | H0YGX0 | transferase.activity; protein.phosphorylat; protein.kinase.a |
| chr11 | 94276724 | upstr | F5H116 | piRNA.binding; DNA.methylation.invo; piRNA.metabolic.proc; m |
| chr18 | 44542946 | exon | K7EPQ6 | ATP.binding; protein.binding; four-way.junction.he; nucleosi |

The predictive power of each CpG site may be ranked by the P-value of the individual sites in differentiating between non-CP and CP patients. Table 5 ranks the top 40 sites by P-value.

TABLE 5

| CpG Site | logFC | FDR adj P-val | Response | Gene | Description |
|---|---|---|---|---|---|
| chr3.0170984628 | 1.72 | 5.54e-33 | up | ENSG00000154310 | NP.001155035 |
| chr12.0113912958 | −1.49 | 1.26e-25 | down | ENSG00000257935 | NP.071758 |
| chr5.0021333675 | 5.11 | 3.26e-24 | up | ENSG00000249359 | — |
| chr1.0000565262 | −3.69 | 2.81e-23 | down | ENSG00000230021 | XP.003403763 |
| chr18.0059002180 | 1.22 | 7.40e-20 | up | ENSG00000101542 | NP.114097 |
| chr22.0021469408 | 2.02 | 8.36e-19 | up | ENSG00000169668 | — |
| chr16.0002689275 | −1.31 | 8.83e-18 | down | ENSG00000205918 | — |
| chr12.0006446797 | 1.83 | 9.62e-18 | up | F5H6Z2 | — |
| chr15.0028343785 | −1.24 | 8.96e-17 | down | ENSG00000104044 | — |
| chr6.0161033995 | 1.10 | 1.60e-16 | up | ENSG00000198670 | NP.005568 |
| chr18.0004068725 | 1.30 | 1.60e-16 | up | A0A0A0MTP4 | — |
| chr12.0001704443 | −1.23 | 1.89e-16 | down | F5GYM2 | — |
| chr11.0013984067 | 1.25 | 4.52e-16 | up | ENSG00000152268 | — |
| chr2.0243078103 | 1.49 | 1.40e-15 | up | ENSG00000220804 | NP.776182 |
| chr19.0020188618 | 1.14 | 1.68e-15 | up | ZNF90 | zinc finger protein 90 |
| chr6.0169653176 | −1.14 | 1.81e-15 | down | Q5RI53 | — |
| chr6.0007394523 | 1.11 | 1.82e-15 | up | E9PFQ8 | — |
| chr6.0117587149 | 1.15 | 5.81e-15 | up | ENSG00000170162 | NP.872586 |
| chr12.0058160354 | 1.26 | 5.81e-15 | up | V9GYP0 | — |
| chr13.0033220266 | −1.18 | 1.67e-14 | down | A9IYQ1 | — |
| chr12.0002113367 | −1.07 | 2.95e-14 | down | ENSG00000151065 | — |
| chr13.0114547107 | −1.06 | 2.99e-14 | down | ENSG00000183087 | NP.001137418 |
| chr18.0029214163 | 1.16 | 7.46e-14 | up | J3QQY9 | — |
| chr11.0071160096 | −1.10 | 2.45e-13 | down | E9PRL8 | — |
| chr3.0182928999 | 1.07 | 4.68e-13 | up | C9J326 | — |
| chr6.0150284688 | 1.20 | 6.75e-13 | up | ULBP1 | UL16 binding protein 1 |
| chr11.0093475384 | 1.07 | 1.35e-12 | up | ENSG00000166012 | — |
| chr16.0030886974 | −1.15 | 1.35e-12 | down | ENSG00000099385 | NP.004756 |
| chr17.0007975795 | 1.18 | 1.79e-12 | up | ALOX12B | arachidonate 12-lipoxygenase |
| chr6.0167001337 | 1.09 | 1.85e-12 | up | ENSG00000071242 | — |
| chr22.0038022735 | 0.974 | 2.08e-12 | up | ENSG00000100083 | NP.001001561 |
| chr12.0108079458 | −1.26 | 2.33e-12 | down | B4DJV5 | — |
| chr11.0000188098 | −0.94 | 2.70e-12 | down | E9PQJ6 | — |
| chr3.0147130343 | −1.04 | 3.41e-12 | down | ZIC1 | Zic family member 1 |
| chr13.0050656618 | −1.40 | 4.13e-12 | down | ENSG00000231607 | — |
| chr3.0197687858 | −1.01 | 4.36e-12 | down | F8WCE5 | — |
| chr6.0149354426 | 0.950 | 4.88e-12 | up | US | uronyl 2-sulfotransferase |
| chr6.0026235050 | 1.06 | 5.30e-12 | up | HIST1H1D T | histone cluster 1 H1 family me |
| chr17.0011145564 | −0.92 | 5.45e-12 | down | V9GZ25 | — |
| chr9.0033025770 | −0.92 | 8.48e-12 | down | ENSG00000086061 | — |

TABLE 6
Top 1000 CpG Sites. Rank ordered listing of CpG sites contributing
the most to the ordinate discrimination in the NMDS analysis.

| Blood | | Muscle | | Satellite | |
|---|---|---|---|---|---|
| CHR | POS | CHR | POS | CHR | POS |
| chr12 | 108079458 | chr16 | 2184051 | chr18 | 12884805 |
| chr12 | 6446797 | chr15 | 72956969 | chr13 | 57713776 |
| chr12 | 6445005 | chr12 | 62584454 | chr13 | 93896533 |
| chr13 | 33220266 | chr14 | 74179572 | chr20 | 60998493 |
| chr12 | 1704443 | chr12 | 5019004 | chr20 | 5829089 |
| chr6 | 169653176 | chr20 | 60998493 | chr16 | 2176197 |
| chr18 | 4068725 | chr16 | 67969342 | chr17 | 18941025 |
| chr12 | 58160354 | chr6 | 134495313 | chr20 | 48755321 |
| chr22 | 33197965 | chr14 | 35590507 | chr12 | 8234476 |
| chr11 | 71160096 | chr17 | 32964775 | chr11 | 67044849 |
| chr3 | 197687858 | chr16 | 14818378 | chr14 | 96728320 |
| chr18 | 3771925 | chr17 | 18941025 | chr14 | 24615469 |
| chr6 | 150284688 | chr13 | 114105414 | chr13 | 114107632 |
| chr3 | 147130343 | chr12 | 8234476 | chr20 | 60852455 |
| chr18 | 29214163 | chr14 | 24615469 | chr12 | 105114308 |
| chr19 | 20188618 | chr13 | 28195784 | chr15 | 92982723 |
| chr13 | 28497262 | chr20 | 61049813 | chr12 | 57608666 |
| chr7 | 130353922 | chr6 | 150284688 | chr14 | 24878082 |
| chr6 | 7394523 | chr12 | 129079508 | chr15 | 69516868 |
| chr19 | 40723542 | chr22 | 42915052 | chr12 | 662536 |
| chr15 | 99344316 | chr16 | 12020100 | chr20 | 61976483 |
| chr11 | 32112972 | chr20 | 43513649 | chr12 | 83436417 |
| chr11 | 188098 | chr3 | 111280409 | chr17 | 19045779 |
| chr12 | 121082579 | chr12 | 105008942 | chr17 | 53343752 |
| chr17 | 11145564 | chr14 | 103987474 | chr18 | 11747918 |
| chr20 | 5081567 | chr22 | 37897522 | chr12 | 25101805 |
| chr3 | 182928999 | chr12 | 105114308 | chr19 | 48673525 |
| chr12 | 5019351 | chr17 | 65040349 | chr20 | 36915727 |
| chr13 | 93896533 | chr17 | 19045779 | chr22 | 42915052 |
| chr11 | 32113404 | chr13 | 28196417 | chr12 | 67663301 |
| chr17 | 7975795 | chr20 | 37356960 | chr3 | 111280409 |
| chr16 | 58000211 | chr18 | 499464 | chr16 | 21610035 |
| chr18 | 57357804 | chr17 | 73029804 | chr13 | 28550394 |
| chr11 | 6633681 | chr3 | 197676776 | chr13 | 33765016 |
| chr6 | 26235050 | chr3 | 59803935 | chrX | 49178276 |
| chr12 | 69005196 | chr16 | 19728830 | chr14 | 104190006 |
| chr16 | 50582980 | chr3 | 119422009 | chr14 | 50108699 |
| chr12 | 4758511 | chr6 | 139013304 | chr12 | 129065948 |
| chr11 | 94276724 | chr14 | 99984633 | chr12 | 62584454 |
| chr18 | 44542946 | chr11 | 22647259 | chr14 | 104497547 |
| chr20 | 48552544 | chr14 | 88459020 | chr12 | 6334026 |
| chr2 | 200819443 | chr12 | 125026886 | chr17 | 73510671 |
| chr18 | 77267918 | chr20 | 13971342 | chr12 | 123753032 |
| chr13 | 21141519 | chr11 | 70664922 | chr12 | 32908207 |
| chr16 | 48639869 | chr15 | 40886106 | chr11 | 123045794 |
| chr16 | 23706114 | chr17 | 7975795 | chr12 | 49484577 |
| chr12 | 5019004 | chr17 | 26166996 | chr17 | 19046425 |
| chr13 | 96206439 | chr6 | 39029929 | chr14 | 100005173 |
| chr20 | 43513649 | chr15 | 93351469 | chr13 | 99650742 |
| chr11 | 71951989 | chr12 | 9103333 | chr5 | 169695009 |
| chr3 | 32022182 | chr9 | 131710795 | chr20 | 31128317 |
| chr11 | 93063959 | chr12 | 58088346 | chr3 | 49044952 |
| chr5 | 115178530 | chr14 | 24900076 | chr13 | 96137813 |
| chr20 | 633169 | chr20 | 55974298 | chr3 | 122711856 |
| chr6 | 149354426 | chr6 | 139349539 | chr14 | 24900076 |
| chr6 | 26596052 | chr12 | 6931525 | chr18 | 12254395 |
| chr19 | 920756 | chr13 | 108103040 | chr12 | 1742005 |
| chr14 | 45431943 | chr11 | 796607 | chr20 | 31129482 |
| chr22 | 30821443 | chr5 | 65124116 | chr18 | 77209053 |
| chr15 | 60302054 | chr12 | 8662310 | chr15 | 27574055 |
| chr3 | 42740649 | chr13 | 57713776 | chr11 | 8255902 |
| chr6 | 145853785 | chr12 | 49484577 | chr11 | 2408146 |
| chr8 | 68877277 | chr18 | 12885184 | chr6 | 1313218 |
| chr3 | 12883119 | chr17 | 73042810 | chr3 | 48647168 |
| chr11 | 66312805 | chr14 | 58638067 | chr14 | 104119867 |
| chr12 | 54409525 | chr12 | 49208971 | chr12 | 103344694 |
| chr3 | 159563262 | chr10 | 23479600 | chr19 | 33896553 |
| chr14 | 24584488 | chr16 | 2184447 | chr16 | 62003103 |
| chr15 | 22052629 | chr22 | 24816977 | chr16 | 87893023 |
| chr12 | 5992325 | chr17 | 11145564 | chr15 | 45927689 |
| chr16 | 4817451 | chr19 | 15580788 | chr20 | 52825772 |
| chr16 | 2176197 | chr12 | 95038500 | chr15 | 60297395 |
| chr11 | 67204130 | chr16 | 1822579 | chr14 | 38071393 |
| chr11 | 65688012 | chr16 | 88716590 | chr13 | 107176083 |
| chr15 | 62359382 | chr12 | 119596461 | chr14 | 50108792 |
| chr17 | 48172954 | chr12 | 50236157 | chr15 | 21059321 |
| chr15 | 77713456 | chr20 | 60985334 | chr20 | 41534983 |
| chr14 | 80170888 | chr5 | 142150994 | chr20 | 61000773 |
| chr16 | 53538293 | chr12 | 58232795 | chr15 | 90964193 |
| chr12 | 6310543 | chr11 | 122933736 | chr19 | 14089299 |
| chr15 | 21071194 | chr11 | 63932756 | chr15 | 41794568 |
| chr18 | 74204568 | chr15 | 27604283 | chr12 | 96253081 |
| chr17 | 4850081 | chr17 | 73042472 | chr15 | 57680681 |
| chr16 | 14818378 | chr13 | 20711116 | chr18 | 44547036 |
| chr3 | 110791724 | chr3 | 132136984 | chr12 | 121647855 |
| chr16 | 70557879 | chr5 | 134094725 | chr16 | 31193471 |
| chr6 | 39174327 | chr5 | 50681152 | chr6 | 26157100 |
| chr16 | 70834848 | chr18 | 25757350 | chr14 | 93019588 |
| chr2 | 112826693 | chr14 | 74770110 | chr15 | 72946822 |
| chr18 | 11986236 | chr20 | 31330621 | chr18 | 44542946 |
| chr3 | 147128690 | chr6 | 143859047 | chr20 | 61867349 |
| chr12 | 116710705 | chr16 | 66864962 | chr17 | 11390406 |
| chr8 | 97166862 | chr14 | 100626499 | chr13 | 96130928 |
| chr22 | 37977772 | chr16 | 9185813 | chr11 | 63932756 |
| chr12 | 5992812 | chr11 | 61249527 | chr17 | 31110221 |
| chr5 | 149524459 | chr12 | 16064519 | chr17 | 38475164 |
| chr18 | 10938440 | chr22 | 43627738 | chr12 | 65671739 |
| chr11 | 124823462 | chr12 | 96428593 | chr5 | 13939870 |
| chr3 | 197676776 | chr20 | 61428843 | chr18 | 44551594 |
| chr13 | 96206771 | chr20 | 25834932 | chr16 | 2478353 |
| chr11 | 1776846 | chr14 | 58618103 | chr12 | 57985699 |
| chr6 | 169651645 | chr2 | 3381822 | chr14 | 100040905 |
| chr14 | 103575195 | chr11 | 13690984 | chr14 | 91842116 |
| chr18 | 43419102 | chr6 | 56716681 | chr15 | 75164896 |
| chr9 | 21967489 | chr12 | 106696764 | chr18 | 56391319 |
| chr18 | 13895717 | chr15 | 39873517 | chr14 | 23070923 |
| chr14 | 64687513 | chr14 | 45605573 | chr12 | 113902624 |
| chr17 | 40610021 | chr12 | 48214000 | chr11 | 2800586 |
| chr14 | 24906444 | chr17 | 15902161 | chr16 | 70734806 |
| chr6 | 10695540 | chr20 | 32011265 | chr11 | 35641938 |
| chr17 | 80272432 | chr20 | 25254024 | chr16 | 2004921 |
| chr22 | 39417803 | chr12 | 12764809 | chr14 | 76178021 |
| chr12 | 96429846 | chr20 | 60852455 | chr20 | 62173702 |
| chr11 | 67034843 | chr13 | 28497262 | chr12 | 124964851 |
| chr2 | 153450964 | chr6 | 73879011 | chr5 | 124079989 |
| chr14 | 93125680 | chr12 | 118540534 | chr16 | 48419832 |
| chr5 | 170869818 | chr18 | 44556541 | chr13 | 52194967 |
| chr2 | 38978853 | chr5 | 168726909 | chr22 | 31890427 |
| chr18 | 10483180 | chr17 | 19046425 | chr12 | 12016676 |
| chr20 | 44639931 | chr5 | 79378854 | chr22 | 20244288 |
| chr20 | 20434395 | chr22 | 39410193 | chr11 | 7563239 |
| chr19 | 56616939 | chr16 | 4290800 | chr12 | 58021713 |
| chr14 | 42075406 | chr6 | 167765060 | chr12 | 123753445 |
| chr11 | 20784643 | chr2 | 129063272 | chr20 | 48727980 |
| chr18 | 20514217 | chr6 | 3742885 | chr11 | 821322 |
| chr11 | 65744445 | chr10 | 26501357 | chr12 | 20568666 |
| chr16 | 83967135 | chr12 | 43944977 | chr13 | 21286449 |
| chr20 | 31058476 | chr15 | 78287064 | chr14 | 34406142 |
| chr20 | 61584448 | chr5 | 129242435 | chr12 | 18457486 |
| chr3 | 50230579 | chr12 | 53044536 | chr16 | 19728830 |
| chr11 | 68672559 | chr16 | 50746190 | chr12 | 6450539 |
| chr3 | 184278878 | chr15 | 75967934 | chr18 | 61160438 |
| chr15 | 62360962 | chr14 | 34720964 | chr14 | 96002525 |
| chr12 | 103980970 | chr15 | 38545505 | chr3 | 16419520 |
| chr17 | 79800852 | chr2 | 73299016 | chr3 | 13920075 |
| chr12 | 19398518 | chr12 | 96429846 | chr20 | 18447458 |
| chr17 | 53343752 | chr20 | 55800172 | chr12 | 129072067 |
| chr12 | 118540949 | chr17 | 26989567 | chr13 | 40149904 |
| chr22 | 31886188 | chr11 | 126367330 | chr12 | 124196312 |
| chr9 | 125674963 | chr20 | 40822059 | chr11 | 2405177 |
| chr15 | 80452978 | chr6 | 78172408 | chr13 | 95256511 |
| chr3 | 39449150 | chr3 | 189791239 | chr13 | 47191668 |
| chr3 | 42646477 | chr19 | 18366297 | chr3 | 189791239 |
| chr13 | 47250475 | chr12 | 57639448 | chr2 | 10200647 |
| chr3 | 42262164 | chr20 | 633169 | chr20 | 61428843 |
| chr11 | 10229941 | chr2 | 27273727 | chr18 | 46475320 |
| chr16 | 30104676 | chr20 | 24450876 | chr16 | 77912976 |
| chr18 | 50319582 | chr5 | 176766295 | chr20 | 8790229 |
| chr13 | 111837470 | chr5 | 172572092 | chr6 | 75912320 |
| chr11 | 65486827 | chr12 | 7342661 | chr12 | 57602992 |
| chr17 | 4440254 | chr5 | 106822941 | chr5 | 106822941 |
| chr12 | 8662310 | chr3 | 196750460 | chr3 | 196595774 |
| chr11 | 100045994 | chr8 | 145017702 | chr3 | 147130343 |
| chr12 | 114348940 | chr5 | 142054203 | chr17 | 28803808 |
| chr12 | 119419907 | chr6 | 169653572 | chr14 | 72980894 |
| chr3 | 184081196 | chr2 | 73518897 | chr12 | 58145283 |
| chr2 | 63279693 | chr6 | 158019037 | chr5 | 11535023 |
| chr19 | 48673525 | chr20 | 32407915 | chr11 | 70369651 |
| chr12 | 5985138 | chr13 | 32984305 | chr17 | 73042810 |
| chr11 | 75139544 | chr12 | 124964851 | chr9 | 34380008 |
| chr20 | 43513977 | chr18 | 43419097 | chr12 | 12764809 |
| chr12 | 104218860 | chr14 | 24878082 | chr11 | 123016467 |
| chr14 | 103396312 | chr16 | 4975267 | chr16 | 89778878 |
| chr6 | 73676215 | chr11 | 57334814 | chr13 | 26538260 |
| chr16 | 89785669 | chr16 | 62003103 | chr12 | 54424611 |
| chr13 | 99737576 | chr14 | 103575195 | chr2 | 73518897 |
| chr7 | 21466431 | chr14 | 74708903 | chr22 | 32954811 |
| chr17 | 79881668 | chr13 | 110951025 | chr5 | 177199729 |
| chr18 | 7116462 | chr22 | 50747228 | chr16 | 680326 |
| chr16 | 23653028 | chr9 | 113798881 | chr16 | 775102 |
| chr3 | 48514843 | chr19 | 46366124 | chr12 | 112226092 |
| chr2 | 74777409 | chr12 | 57602992 | chr12 | 123319333 |
| chr18 | 14129770 | chr2 | 203777474 | chr9 | 79793512 |
| chr18 | 21768319 | chr12 | 121648196 | chr9 | 37001589 |
| chr9 | 134056175 | chr6 | 10163569 | chr20 | 7905990 |
| chr8 | 11415889 | chr16 | 84404119 | chr17 | 64333731 |
| chr3 | 39425054 | chr17 | 61778909 | chr14 | 52478367 |
| chr19 | 39832370 | chr12 | 58145283 | chr20 | 31150534 |
| chr11 | 74304021 | chr11 | 123431994 | chr12 | 128790228 |
| chr13 | 32663317 | chr12 | 49727102 | chr12 | 31881631 |
| chr15 | 45927689 | chr11 | 64511391 | chr18 | 57193836 |
| chr11 | 102448364 | chr12 | 96253081 | chr20 | 5748204 |
| chr3 | 45837192 | chr6 | 157855779 | chr11 | 70384416 |
| chr11 | 118230644 | chr20 | 30030504 | chr13 | 33220266 |
| chr11 | 48109991 | chr15 | 40014219 | chr12 | 96116260 |
| chr16 | 21610035 | chr17 | 28705169 | chr12 | 120884574 |
| chr3 | 112013079 | chr20 | 2739975 | chr18 | 4068725 |
| chrX | 48648229 | chr15 | 53833309 | chr3 | 20088712 |
| chr11 | 63246601 | chr16 | 4987613 | chr19 | 7038916 |
| chr3 | 13894328 | chr15 | 39873937 | chr12 | 109870635 |
| chr11 | 130320107 | chr17 | 31110221 | chr16 | 583839 |
| chr12 | 53460222 | chr20 | 62150177 | chr20 | 40172803 |
| chr16 | 77953169 | chr13 | 96137813 | chr20 | 55970900 |
| chr14 | 71108090 | chr6 | 108436542 | chr2 | 218771815 |
| chr22 | 42914550 | chr16 | 77912976 | chr3 | 124501859 |
| chr13 | 94830546 | chr15 | 75943842 | chr14 | 51134117 |
| chr6 | 117997665 | chr11 | 57308986 | chr16 | 84042148 |
| chr11 | 65308645 | chr16 | 810757 | chr20 | 3139415 |
| chr11 | 74303282 | chr14 | 104073702 | chr6 | 16145414 |
| chr8 | 22457903 | chr19 | 46993480 | chr11 | 62494490 |
| chr19 | 50919627 | chr14 | 96857563 | chr15 | 89182360 |
| chr6 | 35420737 | chr17 | 77764398 | chr12 | 113635055 |
| chr20 | 40848410 | chr15 | 59668078 | chr12 | 10243158 |
| chr18 | 46444366 | chr12 | 58121508 | chr15 | 59599521 |
| chr15 | 56544942 | chr18 | 20514217 | chr12 | 64846499 |
| chr16 | 75130142 | chr12 | 20568666 | chr18 | 12254784 |
| chr14 | 105525484 | chr5 | 168279190 | chr12 | 83436758 |
| chr13 | 92454487 | chr3 | 48884826 | chr15 | 67442588 |
| chr3 | 195946597 | chr20 | 7905990 | chr20 | 24450876 |
| chr19 | 13051211 | chr17 | 19620263 | chr15 | 84912101 |
| chr11 | 35683582 | chr12 | 117822199 | chr3 | 149299399 |
| chr22 | 35724388 | chr22 | 39716213 | chr12 | 13354616 |
| chr22 | 36769509 | chr12 | 49938027 | chr22 | 38303393 |
| chr16 | 85650395 | chr5 | 149669433 | chr14 | 72649687 |
| chr6 | 167786484 | chr14 | 105154994 | chr13 | 28497621 |
| chr16 | 28884493 | chr12 | 65672082 | chr2 | 217557616 |
| chr13 | 24839413 | chr3 | 21787630 | chr17 | 47021520 |
| chr22 | 36751194 | chr6 | 108478563 | chr15 | 79070483 |
| chr13 | 111807510 | chr6 | 111136859 | chr15 | 43906817 |
| chr13 | 103250163 | chr14 | 104497547 | chr5 | 128430539 |
| chr3 | 4874239 | chr6 | 78172784 | chr14 | 23057952 |
| chr3 | 179041743 | chr13 | 39262227 | chr13 | 36668831 |
| chr12 | 27821943 | chr15 | 72957488 | chr2 | 20250672 |
| chr12 | 48197110 | chr14 | 96002525 | chr16 | 87889666 |
| chr12 | 62585750 | chr11 | 130320107 | chr2 | 69239871 |
| chr6 | 7725842 | chr15 | 75978879 | chr6 | 154640863 |
| chr18 | 59948461 | chr15 | 81426597 | chr13 | 73355806 |
| chr12 | 6981014 | chr20 | 48553244 | chr14 | 69137081 |
| chr10 | 70939275 | chr20 | 61447334 | chr12 | 54415147 |
| chr18 | 29340825 | chr12 | 124854907 | chr13 | 94965396 |
| chr3 | 184304408 | chr15 | 67417860 | chr2 | 29850455 |
| chr6 | 169041074 | chr16 | 87893023 | chr14 | 70081079 |
| chr12 | 72149118 | chr15 | 71040854 | chr17 | 77908058 |
| chr12 | 111625057 | chr17 | 15168099 | chr3 | 138666240 |
| chr18 | 61160438 | chr14 | 105260982 | chr5 | 177181499 |
| chr3 | 138403527 | chr11 | 69458502 | chr20 | 524075 |
| chr3 | 6946725 | chr18 | 12883118 | chr18 | 49866895 |
| chr14 | 93074566 | chr5 | 153418042 | chr15 | 69452943 |
| chr13 | 48612261 | chr6 | 26597382 | chr22 | 38202875 |
| chr6 | 127836074 | chr15 | 101866025 | chr18 | 10740861 |
| chr3 | 183945284 | chr2 | 236401911 | chr11 | 70397632 |
| chr11 | 64643975 | chr11 | 7563239 | chr17 | 15902161 |
| chr6 | 90061855 | chr22 | 43670194 | chr14 | 100020456 |
| chr17 | 46125527 | chr9 | 130829697 | chr2 | 69171202 |
| chr11 | 68848057 | chr18 | 49902983 | chr13 | 28366108 |
| chr12 | 108079829 | chr13 | 28496058 | chr18 | 12912338 |
| chr6 | 145865884 | chr12 | 121124599 | chr13 | 52202765 |
| chr14 | 96893304 | chr2 | 237479179 | chr12 | 58003396 |
| chr8 | 11415272 | chr2 | 159651808 | chr14 | 79032507 |
| chr9 | 113785563 | chr11 | 113953207 | chr15 | 101866025 |
| chr3 | 194407700 | chr11 | 1474987 | chr16 | 20444478 |
| chr3 | 108203622 | chr16 | 84902774 | chr2 | 130738212 |
| chr11 | 124767781 | chr22 | 17971431 | chr12 | 110563292 |
| chr11 | 31841691 | chr12 | 53400235 | chr12 | 117756576 |
| chr11 | 17553236 | chr13 | 42032424 | chr12 | 122235135 |
| chr14 | 99984633 | chr5 | 75698439 | chr14 | 20925176 |
| chr17 | 15139831 | chr3 | 140795796 | chr12 | 124965292 |
| chr2 | 192015446 | chr16 | 22220811 | chr11 | 68181416 |
| chr3 | 14692909 | chr13 | 46356967 | chr14 | 103576584 |
| chr16 | 48468530 | chr12 | 15941496 | chr20 | 18269147 |
| chr16 | 28374665 | chr13 | 41364285 | chr13 | 79194483 |
| chr7 | 120969027 | chr6 | 10842909 | chr6 | 46597757 |
| chr14 | 103864937 | chr5 | 76932016 | chr22 | 46344039 |
| chr20 | 3761969 | chr6 | 168843001 | chr12 | 4810242 |
| chr6 | 43244069 | chr12 | 63543766 | chr2 | 23747265 |
| chr15 | 102042815 | chr12 | 117674458 | chr14 | 24566926 |
| chr14 | 50154896 | chr12 | 98911447 | chr16 | 58069921 |
| chr6 | 15597103 | chr2 | 242147702 | chr12 | 104156552 |
| chr17 | 19028901 | chr20 | 54580489 | chr20 | 43513649 |
| chr8 | 145010868 | chr15 | 44486309 | chr2 | 23718617 |
| chr16 | 4942925 | chr18 | 77166042 | chr15 | 79576380 |
| chr16 | 50583740 | chr13 | 36050303 | chr9 | 97889694 |
| chr16 | 67841171 | chr11 | 111957761 | chr12 | 124811910 |
| chr14 | 95658153 | chr15 | 74364839 | chr12 | 6331831 |
| chr3 | 197687709 | chr12 | 56115631 | chr2 | 102423165 |
| chr19 | 46992651 | chr13 | 110951418 | chr12 | 5678978 |
| chr17 | 72958021 | chr14 | 24641438 | chr4 | 101719592 |
| chr16 | 216561 | chr17 | 76446984 | chr11 | 1035109 |
| chr6 | 160402911 | chr13 | 110874842 | chr14 | 78870082 |
| chr3 | 101280900 | chr11 | 63304614 | chr17 | 76888499 |
| chr3 | 42653997 | chr14 | 105934555 | chr12 | 122296446 |
| chr6 | 149141140 | chr11 | 67808203 | chr12 | 124951222 |
| chr3 | 145878214 | chr20 | 52825772 | chr6 | 116464670 |
| chr3 | 31577139 | chr15 | 69516868 | chr14 | 101195354 |
| chr17 | 2241533 | chr12 | 95490776 | chr20 | 61370545 |
| chr14 | 34931900 | chr11 | 3664603 | chrX | 49359038 |
| chr14 | 104038779 | chr6 | 35474148 | chr13 | 39396050 |
| chr3 | 156193747 | chr14 | 68286442 | chr22 | 25600824 |
| chr3 | 183256169 | chr3 | 145879316 | chr15 | 67439562 |
| chr20 | 31124002 | chr16 | 89477757 | chr12 | 95457527 |
| chr14 | 50236376 | chr6 | 100062881 | chr19 | 11090860 |
| chr3 | 53189890 | chr20 | 18036354 | chr20 | 44481703 |
| chr7 | 111845429 | chr15 | 100654513 | chr5 | 1875877 |
| chr11 | 70953873 | chr12 | 57472496 | chr20 | 62150117 |
| chr14 | 50086676 | chr16 | 55434128 | chr18 | 44298549 |
| chr19 | 30030719 | chr2 | 101736025 | chr3 | 57582687 |
| chr12 | 7052710 | chr14 | 38091621 | chr11 | 122674083 |
| chr22 | 33384015 | chr20 | 20433328 | chr13 | 100623759 |
| chr13 | 50715075 | chr12 | 124951222 | chr16 | 4975267 |
| chr12 | 108080465 | chr9 | 139686019 | chr11 | 6633681 |
| chr18 | 3604915 | chr15 | 75560521 | chr22 | 41613414 |
| chr6 | 145852809 | chr12 | 85672520 | chr2 | 10545065 |
| chr3 | 197677134 | chr18 | 56888466 | chr5 | 11534641 |
| chr22 | 37055690 | chr15 | 92982723 | chr9 | 138649349 |
| chr12 | 112855876 | chr2 | 130738212 | chr12 | 5992812 |
| chr11 | 95823245 | chr14 | 103984664 | chr20 | 2606134 |
| chr10 | 102985949 | chr22 | 24952038 | chr17 | 7388870 |
| chr11 | 63769700 | chr6 | 31939322 | chr2 | 223550141 |
| chr6 | 31913250 | chr15 | 79101067 | chr16 | 89575325 |
| chr12 | 58166267 | chr14 | 91818778 | chr18 | 7062524 |
| chr6 | 158988252 | chr16 | 88555614 | chr22 | 19900000 |
| chr7 | 16685981 | chr12 | 83436417 | chr18 | 13076943 |
| chr11 | 65559288 | chr14 | 104061099 | chr12 | 53298968 |
| chr3 | 5021905 | chr18 | 4068725 | chr15 | 59597553 |
| chr18 | 5288345 | chr16 | 89449669 | chr14 | 97264316 |
| chr8 | 145017295 | chr16 | 21610035 | chr5 | 7824030 |
| chr14 | 100008656 | chr18 | 12254784 | chr10 | 80833721 |
| chr3 | 197678316 | chr12 | 94543824 | chr16 | 84054247 |
| chr6 | 151710123 | chr15 | 92618152 | chr12 | 1905324 |
| chr19 | 33932366 | chr5 | 177548979 | chr13 | 43597331 |
| chr12 | 111052410 | chr8 | 22457514 | chr11 | 70347889 |
| chr11 | 61684309 | chr17 | 76919887 | chr14 | 58618103 |
| chr11 | 309045 | chr16 | 89976937 | chr16 | 2207743 |
| chr14 | 38069872 | chr5 | 141489677 | chr20 | 43514173 |
| chr20 | 31053633 | chr20 | 60961653 | chr17 | 76917960 |
| chr18 | 77289073 | chr12 | 121092873 | chr12 | 123414419 |
| chr11 | 47437823 | chr16 | 31193471 | chr20 | 62150177 |
| chr17 | 1201795 | chr20 | 31033712 | chr9 | 124988967 |
| chr12 | 122244256 | chr6 | 99281500 | chr6 | 116421257 |
| chr16 | 86545651 | chr11 | 77185588 | chr18 | 74616984 |
| chr13 | 28499179 | chr22 | 24503820 | chr18 | 29339341 |
| chr18 | 22867463 | chr11 | 17742994 | chr14 | 50066003 |
| chr14 | 91866859 | chr6 | 108488844 | chr2 | 87226238 |
| chr16 | 17429475 | chr18 | 77209053 | chr14 | 60976611 |
| chr11 | 2935626 | chr12 | 16360748 | chr3 | 117069429 |
| chr20 | 31151987 | chr11 | 31840148 | chr15 | 93351469 |
| chr17 | 21475129 | chr3 | 120626543 | chr6 | 19839518 |
| chr18 | 46462840 | chr17 | 6605234 | chr18 | 57220985 |
| chr20 | 24450876 | chr6 | 16145414 | chr13 | 110897069 |
| chr12 | 124199198 | chr2 | 33701285 | chr14 | 69433207 |
| chr3 | 32443899 | chr22 | 44658343 | chr11 | 74247056 |
| chr12 | 103345691 | chr14 | 91737252 | chr3 | 196676179 |
| chr12 | 13994189 | chr15 | 89422286 | chr19 | 45735345 |
| chr19 | 36288914 | chr17 | 73510671 | chr5 | 80263875 |
| chr3 | 58554460 | chr18 | 11747918 | chr3 | 173303347 |
| chr8 | 52796882 | chr8 | 68865050 | chr12 | 117674458 |
| chr6 | 157863580 | chr12 | 107380638 | chr9 | 131398798 |
| chr20 | 36922850 | chr13 | 28550394 | chr12 | 2404205 |
| chr16 | 89753683 | chr18 | 11097415 | chr18 | 11005668 |
| chr3 | 59758690 | chr11 | 70759954 | chr13 | 50700606 |
| chr12 | 105725767 | chr6 | 144729448 | chr3 | 10231460 |
| chr16 | 75134924 | chr15 | 57802348 | chr16 | 11017915 |
| chr19 | 4019341 | chr12 | 123258900 | chr3 | 14944406 |
| chr2 | 55746336 | chr2 | 201983785 | chr5 | 142150994 |
| chr2 | 85641379 | chr18 | 22931434 | chr5 | 50681152 |
| chr22 | 38390447 | chr18 | 2572410 | chr12 | 1974254 |
| chr6 | 163837639 | chr17 | 79259095 | chr12 | 19566176 |
| chr2 | 232393103 | chr15 | 27478128 | chr12 | 54043247 |
| chr3 | 21762167 | chr6 | 24911803 | chr20 | 25834932 |
| chr8 | 67342312 | chr20 | 19945048 | chr20 | 31330621 |
| chr6 | 108436542 | chr17 | 25886889 | chr12 | 50016510 |
| chr11 | 124734735 | chr2 | 46057068 | chr19 | 4099560 |
| chr14 | 58638067 | chr5 | 169997723 | chr18 | 44547672 |
| chr9 | 77757948 | chr16 | 62065715 | chr20 | 54580426 |
| chr11 | 31844628 | chr5 | 168363687 | chr18 | 72922500 |
| chr10 | 1617717 | chr20 | 48702679 | chr11 | 123064777 |
| chr2 | 110012891 | chr11 | 122849001 | chr3 | 148729673 |
| chr3 | 4535738 | chr22 | 39924996 | chr9 | 43685265 |
| chr19 | 51225935 | chr14 | 105185236 | chr12 | 48360477 |
| chr6 | 163730822 | chr15 | 42170723 | chr12 | 48214000 |
| chrX | 154117632 | chr12 | 83273710 | chr11 | 61463399 |
| chrX | 7198701 | chr2 | 223550141 | chr2 | 46561399 |
| chr20 | 2779590 | chr16 | 84448769 | chr18 | 11982515 |
| chr11 | 122932346 | chr2 | 55746739 | chr17 | 66410246 |
| chr14 | 69364439 | chr3 | 17948642 | chr14 | 103096689 |
| chr17 | 46707012 | chr16 | 68480917 | chr12 | 106512630 |
| chr20 | 644002 | chr9 | 34624650 | chr12 | 49584159 |
| chr20 | 60999519 | chr17 | 8079123 | chr11 | 115115373 |
| chr9 | 6414308 | chr16 | 58060315 | chr2 | 1744014 |
| chr3 | 123117834 | chr15 | 74373338 | chr13 | 36355312 |
| chr20 | 41366127 | chr22 | 19706390 | chr7 | 42275712 |
| chr6 | 5167243 | chr14 | 65400868 | chr17 | 7747505 |
| chr11 | 63582772 | chr19 | 54413591 | chr16 | 1140748 |
| chr3 | 48647168 | chr2 | 69241298 | chr18 | 10463531 |
| chr3 | 77347027 | chr6 | 42145539 | chr8 | 145046909 |
| chr20 | 19884178 | chr14 | 20925176 | chr11 | 130319714 |
| chr20 | 37238504 | chr2 | 218799134 | chr10 | 26504425 |
| chr15 | 102030249 | chr2 | 29419721 | chr6 | 75912800 |
| chr11 | 9597577 | chr9 | 124106790 | chr14 | 34616607 |
| chr6 | 6320434 | chr11 | 370878 | chr14 | 50086676 |
| chr11 | 44330601 | chr16 | 9186173 | chr14 | 50087090 |
| chr5 | 5144081 | chr12 | 113520131 | chr5 | 94956014 |
| chr13 | 21476492 | chr16 | 4974847 | chr6 | 158019037 |
| chr3 | 183536414 | chr16 | 30106990 | chr12 | 53720211 |
| chr17 | 37370611 | chr6 | 42009656 | chr13 | 99737576 |
| chr14 | 64689156 | chr14 | 105887111 | chr15 | 81175425 |
| chr2 | 36584069 | chr20 | 61991803 | chr18 | 46987550 |
| chr14 | 93076860 | chr2 | 68545181 | chr15 | 27663736 |
| chr20 | 8374271 | chr16 | 2091071 | chr12 | 56512395 |
| chr7 | 19151365 | chr22 | 26283851 | chr9 | 18605667 |
| chr16 | 84860840 | chr20 | 61370545 | chr10 | 87623913 |
| chr18 | 44552692 | chr20 | 61456966 | chr3 | 37905107 |
| chr22 | 45152033 | chr12 | 124849639 | chr11 | 1418756 |
| chr6 | 119381919 | chr13 | 29247722 | chr15 | 79601574 |
| chr6 | 71277683 | chr6 | 163235590 | chr14 | 100947216 |
| chr3 | 184292578 | chr22 | 46658755 | chr12 | 95470353 |
| chr18 | 2858929 | chr16 | 88496126 | chr11 | 31840148 |
| chr2 | 159651808 | chr11 | 70670889 | chr16 | 7479261 |
| chr12 | 58158604 | chr16 | 50582980 | chr15 | 33687699 |
| chr5 | 177206553 | chr11 | 377521 | chr17 | 15168099 |
| chr18 | 2978045 | chr18 | 44546902 | chr19 | 46196188 |
| chr17 | 40272106 | chr16 | 433040 | chr15 | 41023492 |
| chr18 | 44552959 | chr11 | 123008365 | chr6 | 163520001 |
| chr3 | 12595771 | chr17 | 53045817 | chr16 | 89560406 |
| chr15 | 52264496 | chr20 | 2675487 | chr20 | 6759498 |
| chr12 | 3145799 | chr6 | 137242330 | chr14 | 45712025 |
| chr3 | 32570450 | chr12 | 121089092 | chr20 | 18622100 |
| chr16 | 87637417 | chr5 | 71604285 | chr18 | 12883118 |
| chr9 | 73028853 | chr12 | 96253464 | chr16 | 84857412 |
| chr13 | 93898907 | chr13 | 43565901 | chr14 | 105753103 |
| chr9 | 126794379 | chr16 | 87725673 | chr19 | 1479590 |
| chr14 | 20256530 | chr12 | 54347057 | chr14 | 61788816 |
| chr13 | 28196417 | chr11 | 8255902 | chr5 | 141489677 |
| chr11 | 75275236 | chr11 | 110001630 | chr20 | 61991803 |
| chr14 | 60976921 | chr12 | 54398945 | chr14 | 80170888 |
| chr22 | 26352862 | chr11 | 70664902 | chr11 | 47470312 |
| chr12 | 82743539 | chr17 | 18221878 | chr11 | 131407861 |
| chr18 | 9924677 | chr9 | 15423618 | chr16 | 67960721 |
| chr11 | 131907196 | chr9 | 131041994 | chr11 | 123032308 |
| chr17 | 63558158 | chr12 | 88973584 | chr3 | 111469507 |
| chr14 | 99698865 | chr9 | 94389711 | chr15 | 67443831 |
| chr6 | 26157100 | chr20 | 524075 | chr14 | 96857563 |
| chr11 | 131793858 | chr11 | 67808133 | chr8 | 145010868 |
| chr22 | 38330213 | chr20 | 45341082 | chr15 | 27482643 |
| chr5 | 166927879 | chr12 | 103980970 | chr18 | 11766735 |
| chr19 | 2090422 | chr20 | 5829089 | chr3 | 33188987 |
| chr12 | 56881793 | chr15 | 75986380 | chr20 | 25254024 |
| chr12 | 96253081 | chr6 | 42035049 | chr11 | 32114158 |
| chr11 | 123448701 | chr2 | 220110584 | chr3 | 10289472 |
| chr20 | 42592237 | chr18 | 4040946 | chr20 | 13971342 |
| chr3 | 32879033 | chr13 | 111297745 | chr3 | 76748606 |
| chr11 | 1771930 | chr15 | 44119388 | chr3 | 195946597 |
| chr12 | 129279448 | chr14 | 105176576 | chr14 | 91862540 |
| chr12 | 6932203 | chr17 | 55968405 | chr14 | 72556804 |
| chr12 | 109026041 | chr12 | 125001363 | chr14 | 100046540 |
| chr3 | 159089122 | chr12 | 58021713 | chr13 | 50735214 |
| chr11 | 111231053 | chr11 | 69175088 | chr16 | 84902774 |
| chr15 | 78406033 | chr20 | 8703238 | chr13 | 50810278 |
| chr16 | 23520796 | chr15 | 90745015 | chr18 | 42403544 |
| chr7 | 44163351 | chr16 | 56445309 | chr14 | 90651458 |
| chr2 | 45232263 | chr11 | 132206308 | chr20 | 37591461 |
| chr16 | 1145419 | chr20 | 2732182 | chr2 | 74780832 |
| chr18 | 50269807 | chr8 | 145010868 | chr16 | 4290800 |
| chr15 | 74364965 | chr6 | 161916305 | chr14 | 24459548 |
| chr19 | 45989930 | chr14 | 103435987 | chr15 | 98986819 |
| chr20 | 31157747 | chr14 | 38093987 | chr2 | 46558320 |
| chr13 | 48894526 | chr15 | 99414436 | chr18 | 10479529 |
| chr5 | 153854505 | chr14 | 102505062 | chr13 | 111298119 |
| chr11 | 122933736 | chr17 | 56024688 | chr14 | 103435987 |
| chr5 | 177763125 | chr6 | 116421257 | chr14 | 72557012 |
| chr2 | 85534352 | chr2 | 176945893 | chr14 | 100032833 |
| chr3 | 184294227 | chr20 | 48179196 | chr14 | 71878448 |
| chr18 | 56941246 | chr19 | 1827105 | chr12 | 2464255 |
| chr18 | 51887566 | chr13 | 74686846 | chr22 | 40899482 |
| chr22 | 23568235 | chr20 | 17661242 | chr11 | 2411869 |
| chr20 | 1370173 | chr10 | 73769075 | chr3 | 192959361 |
| chr17 | 33416862 | chr16 | 70333674 | chr17 | 19035897 |
| chr6 | 135513156 | chr11 | 57282706 | chr13 | 41634896 |
| chr3 | 196227877 | chr17 | 27056178 | chr12 | 103980970 |
| chr16 | 21425800 | chr2 | 9770200 | chr12 | 110438723 |
| chr16 | 723354 | chr20 | 61976483 | chr13 | 76385484 |
| chr22 | 23524799 | chr16 | 2060228 | chr12 | 53958599 |
| chr19 | 38926845 | chr2 | 242665083 | chr11 | 63246601 |
| chr20 | 8224158 | chr14 | 77626094 | chr8 | 133070210 |
| chr14 | 74551060 | chr18 | 43419102 | chr12 | 2307583 |
| chr12 | 101777044 | chr12 | 54389511 | chr13 | 36606864 |
| chr22 | 28315532 | chr15 | 61212366 | chr20 | 60821582 |
| chr17 | 74006497 | chr22 | 32341622 | chr15 | 92511435 |
| chr15 | 74423910 | chr15 | 43903305 | chr11 | 61337443 |
| chr11 | 73034806 | chr16 | 88871463 | chr5 | 37620056 |
| chr3 | 108192015 | chr16 | 7354650 | chr9 | 138599340 |
| chr14 | 95721522 | chr11 | 14541749 | chr12 | 49463725 |
| chrX | 154492497 | chr11 | 62494490 | chr20 | 61049813 |
| chr17 | 73031154 | chr3 | 178866452 | chr20 | 44408318 |
| chr8 | 82633895 | chr16 | 84403863 | chr15 | 68583382 |
| chr18 | 77166371 | chr12 | 124994457 | chr16 | 774394 |
| chr15 | 21046971 | chr16 | 86612198 | chr16 | 89243034 |
| chr14 | 61415347 | chr2 | 171574558 | chr17 | 15394553 |
| chr9 | 72659513 | chr9 | 34380008 | chr3 | 183959590 |
| chr13 | 96205937 | chr20 | 4705306 | chr11 | 62211039 |
| chr2 | 225792999 | chr22 | 46367584 | chr14 | 69403414 |
| chr5 | 174907220 | chr5 | 92956917 | chr5 | 168279190 |
| chr13 | 112724188 | chr2 | 69171202 | chr3 | 59803935 |
| chr11 | 2801887 | chr3 | 56929786 | chr12 | 96253464 |
| chr14 | 20268631 | chr13 | 93879327 | chr14 | 100588329 |
| chr6 | 35479737 | chr3 | 189838858 | chr6 | 168842694 |
| chr13 | 93389680 | chr14 | 104635897 | chr12 | 57505779 |
| chr20 | 61463899 | chr13 | 107186333 | chr12 | 124860589 |
| chr19 | 16482757 | chr20 | 36927337 | chr12 | 57529447 |
| chr7 | 87506431 | chr13 | 110882278 | chr12 | 121176300 |
| chr12 | 57471870 | chr3 | 49066553 | chr18 | 44556402 |
| chr16 | 66460341 | chr15 | 90345633 | chr12 | 54425019 |
| chr20 | 61871027 | chr9 | 96087601 | chr20 | 2488998 |
| chr6 | 13430390 | chr15 | 89182360 | chr17 | 25887321 |
| chr8 | 117779038 | chr12 | 6310543 | chr20 | 398110 |
| chr14 | 34421550 | chr11 | 131618285 | chr2 | 3828507 |
| chr22 | 18647741 | chr14 | 68162520 | chr15 | 71044501 |
| chr15 | 34517834 | chr16 | 11763519 | chr10 | 135088481 |
| chr20 | 31058131 | chr6 | 73866782 | chr14 | 70120944 |
| chr5 | 142369871 | chr3 | 22008166 | chr14 | 36005115 |
| chr16 | 4816546 | chr20 | 43992344 | chr10 | 95327032 |
| chr18 | 11147409 | chr11 | 100812886 | chr17 | 1201795 |
| chr12 | 1974254 | chr16 | 88704921 | chr18 | 77203041 |
| chr15 | 30152204 | chr6 | 4074381 | chr16 | 10906798 |
| chr16 | 75455052 | chr6 | 46293418 | chr5 | 36158752 |
| chr16 | 84871409 | chr17 | 38191484 | chr14 | 96890099 |
| chr22 | 17849502 | chr12 | 48214771 | chr3 | 4536474 |
| chr10 | 80884368 | chr12 | 53459916 | chr18 | 46472755 |
| chr12 | 6894204 | chr3 | 183146049 | chr18 | 72959929 |
| chr3 | 88109356 | chr5 | 80491458 | chr20 | 20421487 |
| chr19 | 20190648 | chr11 | 115228019 | chr16 | 88704921 |
| chr12 | 49245112 | chr18 | 61642608 | chr14 | 96729260 |
| chr5 | 76878190 | chr14 | 30395832 | chr15 | 42566038 |
| chr20 | 61879154 | chr14 | 61706209 | chr11 | 70759954 |
| chr19 | 45607125 | chr9 | 976203 | chr12 | 2167451 |
| chr3 | 58980595 | chr22 | 38202875 | chr19 | 54493114 |
| chr5 | 670411 | chr5 | 177548296 | chr22 | 38300601 |
| chr20 | 19999185 | chr20 | 61637944 | chr16 | 87892303 |
| chr3 | 53286948 | chr6 | 150097548 | chr14 | 100226368 |
| chr10 | 34494305 | chr12 | 4392642 | chr3 | 185230852 |
| chr18 | 44555803 | chr5 | 94956014 | chr16 | 89927438 |
| chr18 | 47448008 | chr20 | 62173702 | chr15 | 79071423 |
| chr16 | 30380713 | chr13 | 52586608 | chr14 | 53155967 |
| chr18 | 72917447 | chr7 | 21744160 | chr3 | 33245973 |
| chr12 | 65152398 | chr19 | 38987939 | chr12 | 5769959 |
| chr8 | 128750864 | chr6 | 150465387 | chr17 | 11356330 |
| chr11 | 66057892 | chr18 | 22929926 | chr14 | 104416308 |
| chr19 | 14096082 | chr16 | 67701464 | chr11 | 69631355 |
| chr14 | 35590507 | chr15 | 74364445 | chr5 | 176766295 |
| chr11 | 63804799 | chr12 | 13353858 | chr9 | 130829697 |
| chr11 | 126335235 | chr12 | 15942959 | chr15 | 33616113 |
| chr19 | 50038397 | chr16 | 25267063 | chr16 | 87653238 |
| chr11 | 124746968 | chr20 | 61428432 | chr12 | 58166267 |
| chr12 | 20575002 | chr11 | 65154531 | chr14 | 101194311 |
| chr14 | 24611126 | chr6 | 148032878 | chr15 | 29644298 |
| chr7 | 6629867 | chr6 | 76105156 | chr15 | 40575311 |
| chr2 | 232648810 | chr16 | 62059434 | chr9 | 117982569 |
| chr17 | 7492315 | chr6 | 161774787 | chr11 | 64623629 |
| chr18 | 74184722 | chr12 | 54387478 | chr11 | 2291427 |
| chr12 | 66276259 | chr19 | 10812454 | chr19 | 42748732 |
| chr2 | 29541112 | chr22 | 46348686 | chr7 | 55089779 |
| chr12 | 14050295 | chr20 | 31162514 | chr11 | 69485231 |
| chr14 | 45432114 | chr16 | 3334273 | chr15 | 65707572 |
| chr11 | 45676931 | chr20 | 57430078 | chr19 | 15108434 |
| chr11 | 124747117 | chr20 | 19899870 | chr12 | 70761688 |
| chr16 | 597813 | chr6 | 96463876 | chr14 | 91881488 |
| chr15 | 78289462 | chr20 | 35723630 | chr6 | 158333116 |
| chr2 | 228382074 | chr13 | 70682432 | chr12 | 48181849 |
| chr15 | 93277652 | chr20 | 25839615 | chr2 | 26943260 |
| chr18 | 43411066 | chr15 | 84912101 | chr12 | 125001363 |
| chr11 | 131701732 | chr3 | 58067906 | chr3 | 148733771 |
| chr3 | 32875020 | chr16 | 597050 | chr18 | 47337882 |
| chr16 | 10007064 | chr16 | 84042148 | chr16 | 29706607 |
| chr16 | 75145905 | chr12 | 124844596 | chr5 | 172753741 |
| chr12 | 27864385 | chr14 | 76102495 | chr18 | 44552959 |
| chr14 | 35591071 | chr13 | 111213203 | chr12 | 32907741 |
| chr8 | 52797197 | chr6 | 149234391 | chr17 | 20909292 |
| chr16 | 433009 | chr14 | 34406142 | chr19 | 50463353 |
| chr15 | 67366968 | chr11 | 111316435 | chr16 | 88806631 |
| chr11 | 46931505 | chr11 | 131392084 | chr5 | 170054036 |
| chr5 | 167532506 | chr6 | 158980660 | chr16 | 87159484 |
| chr18 | 57006092 | chr16 | 67841171 | chr20 | 36839379 |
| chr15 | 90358013 | chr20 | 55975496 | chr5 | 15555589 |
| chr6 | 169049163 | chr15 | 38857458 | chr16 | 57461160 |
| chr11 | 64558464 | chr14 | 69403414 | chr11 | 123008365 |
| chr14 | 24644510 | chr14 | 105165054 | chr10 | 102767357 |
| chr11 | 117687007 | chr20 | 2488998 | chr6 | 90202117 |
| chr13 | 28366049 | chr5 | 141489233 | chr3 | 13899697 |
| chr8 | 145011320 | chr6 | 121758708 | chr22 | 21386178 |
| chr3 | 97645615 | chr14 | 65170734 | chr12 | 25349946 |
| chr20 | 25834932 | chr18 | 72916319 | chr13 | 52283547 |
| chr17 | 76919887 | chr2 | 129027501 | chr12 | 107203023 |
| chr19 | 49535232 | chr6 | 74099405 | chr12 | 27218245 |
| chr8 | 144508378 | chr14 | 58712032 | chr9 | 115916993 |
| chr14 | 78228378 | chr12 | 67663301 | chr14 | 80790843 |
| chr11 | 61313646 | chr15 | 101449971 | chr17 | 20940382 |
| chr22 | 46319798 | chr18 | 2878772 | chr22 | 27009776 |
| chr15 | 57669322 | chr14 | 96857612 | chr12 | 46149812 |
| chr3 | 42743744 | chr16 | 812359 | chr12 | 96602750 |
| chr8 | 8559992 | chr9 | 77567031 | chr16 | 89412525 |
| chr15 | 75986380 | chr5 | 320856 | chr20 | 35516795 |
| chr20 | 61384532 | chr18 | 4254337 | chr18 | 13912474 |
| chr16 | 56442924 | chr20 | 259321 | chr2 | 85443376 |
| chr14 | 69414699 | chr18 | 59728930 | chr19 | 7598550 |
| chr11 | 32455081 | chr5 | 92956738 | chr22 | 31536540 |
| chr11 | 69065008 | chr13 | 93114991 | chr9 | 139433088 |
| chr16 | 75414554 | chr11 | 95976442 | chr18 | 43531720 |
| chr14 | 99994086 | chr12 | 53216725 | chr11 | 61344025 |
| chr2 | 192710945 | chr13 | 70681911 | chr11 | 111475384 |
| chr12 | 49659358 | chr20 | 3778247 | chr12 | 112875260 |
| chr17 | 79894400 | chr14 | 52510545 | chr19 | 608811 |
| chr11 | 72624025 | chr2 | 64067784 | chr15 | 92524072 |
| chr11 | 123008747 | chr18 | 77634030 | chr20 | 23859143 |
| chr14 | 78227825 | chr11 | 115115373 | chr6 | 40553866 |
| chr3 | 152554055 | chr14 | 100005173 | chr20 | 40847575 |
| chr15 | 67388306 | chr2 | 118572504 | chr15 | 90940177 |
| chr14 | 80226992 | chr22 | 19796827 | chr16 | 70746421 |
| chr17 | 7146617 | chr16 | 7137256 | chr20 | 260147 |
| chr2 | 55747731 | chr9 | 21973527 | chr20 | 44458275 |
| chr16 | 627501 | chr16 | 67195677 | chr14 | 105254062 |
| chr16 | 28989716 | chr20 | 23474877 | chr17 | 76893194 |
| chr16 | 25909981 | chr16 | 28894484 | chr2 | 150443909 |
| chr13 | 95260109 | chr6 | 150285021 | chr12 | 5019004 |
| chr8 | 133419735 | chr14 | 97264316 | chr20 | 60983938 |
| chr14 | 95687095 | chr16 | 89930217 | chr2 | 175765980 |
| chr2 | 241636085 | chr17 | 72958021 | chr16 | 2478275 |
| chr3 | 107322520 | chr17 | 46129803 | chr16 | 2757124 |
| chr14 | 105760800 | chr3 | 132072978 | chr20 | 37602417 |
| chr3 | 64671148 | chr11 | 13484802 | chr15 | 31634557 |
| chr16 | 21436223 | chr12 | 19585485 | chr17 | 78967291 |
| chr12 | 1675739 | chr14 | 80132110 | chr2 | 99422979 |
| chr13 | 70681911 | chr12 | 57039757 | chr12 | 105134526 |
| chr11 | 41165217 | chr12 | 2762206 | chr14 | 93020078 |
| chr14 | 74554043 | chr12 | 117316967 | chr19 | 2776127 |
| chr8 | 145017622 | chr5 | 15575251 | chr9 | 111639318 |
| chr19 | 55813389 | chr16 | 55514353 | chr17 | 72333268 |
| chr11 | 121431954 | chr22 | 37710318 | chr2 | 225889464 |
| chr12 | 49584012 | chr13 | 96199517 | chr2 | 218762882 |
| chr5 | 134367354 | chr16 | 89640944 | chr12 | 109878262 |
| chr16 | 88637645 | chr14 | 31890023 | chr18 | 21696228 |
| chr17 | 58466828 | chr20 | 2821334 | chr21 | 46032860 |
| chr17 | 65821666 | chr20 | 61360593 | chr14 | 88741212 |
| chr11 | 134096175 | chr3 | 12883119 | chr16 | 1582320 |
| chr8 | 10568487 | chr20 | 48751441 | chr10 | 124235281 |
| chr14 | 72621904 | chr15 | 90563725 | chr11 | 60893888 |
| chr8 | 141584493 | chr2 | 219647411 | chr11 | 11483238 |
| chr3 | 45626270 | chr9 | 136080790 | chr12 | 2513357 |
| chr19 | 18366297 | chr10 | 98135340 | chr13 | 21291297 |
| chr14 | 92999334 | chr16 | 835346 | chr14 | 93182497 |
| chr15 | 75746111 | chr19 | 38209416 | chr20 | 62153435 |
| chr15 | 53076542 | chr14 | 92359413 | chr12 | 124871301 |
| chr11 | 74953408 | chr11 | 131764597 | chr22 | 33395427 |
| chr12 | 25254118 | chr17 | 9720552 | chr12 | 81545697 |
| chr20 | 31169245 | chr3 | 143691937 | chr5 | 149643466 |
| chr16 | 84857412 | chr6 | 15525504 | chr20 | 41025294 |
| chr11 | 121342283 | chr20 | 36662866 | chr15 | 81187412 |
| chr3 | 45696504 | chr20 | 36916048 | chr20 | 18269858 |
| chr3 | 64672363 | chr6 | 42017070 | chr6 | 42145539 |
| chr16 | 58026944 | chr12 | 123883253 | chr15 | 67989241 |
| chr3 | 119043164 | chr22 | 19022843 | chr19 | 14218840 |
| chr7 | 27211142 | chr20 | 18122250 | chr9 | 139658324 |
| chr16 | 70755102 | chr12 | 72668032 | chr11 | 123006730 |
| chr16 | 54316968 | chr2 | 25097119 | chr15 | 27478128 |
| chr16 | 28518300 | chr18 | 59560869 | chr11 | 20621109 |
| chr11 | 31849286 | chr16 | 67969760 | chr12 | 10242982 |
| chr11 | 67767580 | chr5 | 133863666 | chr12 | 117763391 |
| chr16 | 28660291 | chr12 | 53732109 | chr6 | 139013304 |
| chr19 | 50193737 | chr17 | 77766636 | chr11 | 66315845 |
| chr2 | 176959229 | chr5 | 118789039 | chr6 | 168868993 |
| chr12 | 19588307 | chr6 | 32008451 | chr17 | 1844918 |
| chr13 | 21247259 | chr6 | 47210387 | chr11 | 114042360 |
| chr16 | 66968093 | chr5 | 841665 | chr12 | 124994457 |
| chr17 | 27331604 | chr12 | 2113718 | chr10 | 133949269 |
| chr20 | 470768 | chr5 | 76932335 | chr3 | 4697567 |
| chr3 | 158389938 | chr18 | 9823938 | chr12 | 56661265 |
| chr20 | 37354013 | chr16 | 70723448 | chr11 | 12353078 |
| chr12 | 119772501 | chr19 | 6738430 | chr12 | 83273710 |
| chr15 | 75164896 | chr15 | 59597553 | chr12 | 63544384 |
| chr17 | 73033363 | chr12 | 54390585 | chr5 | 171708946 |
| chr11 | 75274336 | chr2 | 111705850 | chr18 | 77269146 |
| chr5 | 171539911 | chr14 | 57272244 | chr11 | 70917520 |
| chr15 | 23931011 | chr8 | 82632818 | chr2 | 223166989 |
| chr3 | 120742695 | chr11 | 63795251 | chr16 | 50285587 |
| chr17 | 81046973 | chr6 | 394127 | chr20 | 13255338 |
| chr17 | 79101340 | chr8 | 32078317 | chr12 | 58088346 |
| chr10 | 32216359 | chr20 | 48750272 | chr13 | 74262821 |
| chr6 | 168842694 | chr8 | 38307992 | chr18 | 77938254 |
| chr12 | 93322293 | chr14 | 93153308 | chr13 | 94298869 |
| chr6 | 107967104 | chr13 | 36044855 | chr11 | 113962966 |
| chr6 | 159026326 | chr11 | 67007442 | chr18 | 21769731 |
| chr14 | 31419741 | chr14 | 105678272 | chr7 | 1271258 |
| chr19 | 14154146 | chr12 | 20568401 | chr16 | 21845303 |
| chr3 | 56400417 | chr22 | 42912017 | chr9 | 126782799 |
| chr18 | 11749566 | chr10 | 26504425 | chr3 | 39425054 |
| chr5 | 126152447 | chr13 | 80910987 | chr22 | 28192718 |
| chr15 | 101429765 | chr11 | 65341168 | chr12 | 108987690 |
| chr18 | 597538 | chr6 | 31091704 | chr5 | 418941 |
| chr3 | 57029280 | chr6 | 3743561 | chr18 | 77218458 |
| chr12 | 121124138 | chr3 | 160167980 | chr12 | 40460895 |
| chr20 | 524075 | chr12 | 94581301 | chr14 | 103488854 |
| chr14 | 68830387 | chr6 | 26087971 | chr12 | 2707006 |
| chr12 | 124981003 | chr14 | 30395697 | chr17 | 77992241 |
| chr9 | 136567971 | chr11 | 74303282 | chr11 | 64012692 |
| chr18 | 12698326 | chr6 | 18262136 | chr2 | 233390153 |
| chr11 | 63766763 | chr13 | 92898972 | chr20 | 31597415 |
| chr22 | 37965337 | chr17 | 25887321 | chr19 | 40006727 |
| chr10 | 33574870 | chr6 | 82379185 | chr11 | 131764597 |
| chr9 | 139130655 | chr5 | 125937451 | chr19 | 6738430 |
| chr18 | 74184089 | chr6 | 33394110 | chr15 | 61121482 |
| chr9 | 139844692 | chr3 | 123150850 | chr12 | 119596461 |
| chr18 | 77282967 | chr11 | 809763 | chr6 | 159238500 |
| chr12 | 111618854 | chr3 | 50712994 | chr20 | 10445978 |
| chr6 | 162311892 | chr18 | 44298549 | chr14 | 30224715 |
| chr17 | 65357554 | chr10 | 80833721 | chr11 | 68082354 |
| chr15 | 100843573 | chr3 | 182995829 | chr12 | 54424639 |
| chr19 | 37263615 | chr18 | 47087853 | chr13 | 21398086 |
| chr7 | 137619935 | chr3 | 120461953 | chr15 | 99204781 |
| chr19 | 18060689 | chr20 | 61893513 | chr16 | 84404119 |
| chr16 | 75144077 | chr19 | 2675391 | chr20 | 20433328 |
| chr16 | 11768860 | chr19 | 49118462 | chr13 | 30118193 |
| chr15 | 78300153 | chr17 | 17848335 | chr3 | 99679001 |
| chr16 | 87886652 | chr6 | 16240110 | chr17 | 3641758 |
| chr11 | 65788985 | chr15 | 74472301 | chr13 | 47216123 |
| chr5 | 179344322 | chr14 | 103488854 | chr6 | 168843099 |
| chr11 | 126339780 | chr14 | 74709238 | chr14 | 70178796 |
| chr2 | 9564305 | chr2 | 218723774 | chr18 | 61642608 |
| chr12 | 83113033 | chr19 | 22019003 | chr13 | 21288175 |
| chr12 | 49484862 | chr13 | 100637888 | chr3 | 13871726 |
| chr6 | 149102465 | chr16 | 4268743 | chr12 | 118627799 |
| chr2 | 11051912 | chr20 | 61050006 | chr11 | 2404837 |
| chr3 | 79476146 | chr6 | 154624519 | chr18 | 42303796 |
| chr11 | 63783225 | chr12 | 29359874 | chr11 | 77757459 |
| chr11 | 20792020 | chr8 | 42356881 | chr20 | 57830930 |
| chr5 | 156675967 | chr19 | 55691088 | chr14 | 93016108 |
| chr17 | 33324385 | chr22 | 33040841 | chr7 | 21744160 |
| chr17 | 80041603 | chr11 | 131407861 | chr12 | 120241673 |
| chr6 | 158091162 | chr11 | 70760305 | chr5 | 139641109 |
| chr12 | 654654 | chr22 | 37535328 | chr12 | 120268957 |
| chr2 | 231913195 | chr6 | 144606507 | chr3 | 58612895 |
| chr15 | 99458984 | chrX | 49178276 | chr20 | 23860253 |
| chr3 | 53106296 | chr17 | 33759812 | chr20 | 45799615 |
| chr8 | 120669325 | chr15 | 63642428 | chr14 | 54977128 |
| chr17 | 80437321 | chr13 | 100620398 | chr14 | 72106918 |
| chr3 | 155935956 | chr6 | 13625104 | chr19 | 41126191 |
| chr2 | 208632302 | chr15 | 66798030 | chr19 | 18812198 |
| chr13 | 70357073 | chr3 | 64519536 | chr15 | 73617968 |
| chr22 | 41613790 | chr14 | 103996000 | chr20 | 61456818 |
| chr20 | 39969536 | chr2 | 119914029 | chr14 | 77245353 |
| chr7 | 158936628 | chr9 | 127231878 | chr19 | 44555531 |
| chr3 | 32450467 | chr14 | 38344006 | chr19 | 6531828 |
| chr2 | 183731397 | chr3 | 88198086 | chr2 | 121563023 |
| chr18 | 4106607 | chr3 | 120461099 | chr13 | 114321481 |
| chr17 | 29285800 | chr6 | 73974640 | chr14 | 68416082 |
| chr9 | 97890132 | chr20 | 16734749 | chr11 | 125877755 |
| chr6 | 41978335 | chr20 | 13956941 | chr18 | 46447973 |
| chr3 | 57029928 | chr14 | 100233662 | chr10 | 670920 |
| chr3 | 149094816 | chr6 | 100055731 | chr2 | 23812332 |
| chr3 | 6956182 | chr15 | 91422745 | chr13 | 26230574 |
| chr3 | 127348640 | chr6 | 105774191 | chr5 | 74964669 |
| chr4 | 7652024 | chr16 | 70771850 | chr3 | 49170515 |
| chr7 | 121957645 | chr6 | 108487435 | chr16 | 84637283 |
| chr19 | 7984203 | chr6 | 154640863 | chr14 | 100012604 |
| chr2 | 27273727 | chr14 | 103576584 | chr22 | 19036590 |
| chr20 | 44749560 | chr5 | 142316968 | chr18 | 11046430 |
| chr12 | 63277133 | chr6 | 162376364 | chr13 | 110939920 |
| chr11 | 41137301 | chr14 | 42078677 | chr12 | 15870446 |
| chr3 | 21993057 | chr17 | 62040725 | chr18 | 44548050 |
| chr14 | 65019063 | chr5 | 175501676 | chr20 | 523369 |
| chr3 | 183959143 | chr11 | 69065008 | chr16 | 84403863 |
| chr17 | 60879789 | chr2 | 65599066 | chr5 | 419577 |
| chr2 | 212603392 | chr17 | 55953009 | chr14 | 69861077 |
| chr19 | 45575711 | chr12 | 54653293 | chr17 | 9331223 |
| chr19 | 49642506 | chr14 | 60462747 | chr2 | 230499547 |
| chr15 | 45421198 | chr17 | 637070 | chr8 | 10191232 |
| chr16 | 29873147 | chr12 | 2508695 | chr14 | 24728926 |
| chr16 | 5019032 | chr14 | 88692995 | chr18 | 74539147 |
| chr15 | 31653107 | chr14 | 61835153 | chr13 | 80097622 |
| chr11 | 126398134 | chr18 | 11873008 | chr12 | 119575245 |
| chr11 | 68141850 | chr15 | 53858451 | chr14 | 78015650 |
| chr12 | 49391731 | chr12 | 39300318 | chr17 | 74566299 |
| chr16 | 2273434 | chr5 | 176761538 | chr13 | 99997673 |
| chr22 | 38837189 | chr16 | 87713080 | chr4 | 6418422 |
| chr22 | 38479288 | chr14 | 99984701 | chr22 | 23588333 |
| chr6 | 84881773 | chr17 | 76920337 | chr14 | 30395697 |
| chr15 | 90578500 | chr2 | 96010988 | chr14 | 72741187 |
| chr15 | 42844381 | chr12 | 57912738 | chr14 | 76232925 |
| chr16 | 2740054 | chr11 | 124733477 | chrX | 23418321 |
| chr15 | 39872902 | chr7 | 102154954 | chr15 | 31626478 |
| chr3 | 184278810 | chr15 | 99056821 | chr12 | 109796911 |
| chr20 | 61340657 | chr16 | 83960069 | chr12 | 103350479 |
| chr11 | 21317728 | chr12 | 57617584 | chr12 | 3119176 |
| chr17 | 38210783 | chr13 | 26358849 | chr15 | 31690233 |
| chr3 | 124530718 | chr2 | 60984163 | chr19 | 2759241 |
| chr20 | 18035242 | chr14 | 51134117 | chr11 | 370583 |
| chr16 | 12020100 | chr14 | 100020933 | chr12 | 109796872 |
| chr16 | 17454764 | chr16 | 77884642 | chr12 | 111604655 |
| chr13 | 51098489 | chr13 | 70358340 | chr14 | 105945135 |
| chr5 | 5179761 | chr14 | 23901954 | chr22 | 50764059 |
| chr12 | 113902624 | chr3 | 33259743 | chr12 | 114371346 |
| chr6 | 144675770 | chr12 | 11948268 | chr12 | 15366373 |
| chr11 | 89537228 | chr12 | 57524281 | chr14 | 42169831 |
| chr14 | 34640878 | chr16 | 4974413 | chr9 | 138597861 |
| chr5 | 58633250 | chr14 | 91827095 | chr11 | 119236227 |
| chr17 | 1686671 | chr17 | 76888499 | chr6 | 7729757 |
| chr5 | 177811771 | chr3 | 9005400 | chr12 | 53773056 |
| chr6 | 75956744 | chr11 | 89602214 | chr17 | 40832281 |
| chr18 | 67070165 | chr13 | 99918251 | chr2 | 235873378 |
| chr22 | 36783085 | chr2 | 172388050 | chr17 | 70810140 |
| chr6 | 138825229 | chr13 | 47216123 | chr22 | 36960414 |
| chrX | 46616892 | chr8 | 66955958 | chr16 | 2204022 |
| chr16 | 69354749 | chr13 | 27998115 | chr14 | 100069262 |
| chr12 | 132322550 | chr11 | 61215659 | chr20 | 13878367 |
| chr17 | 79970501 | chr12 | 109252608 | chr20 | 31677675 |
| chr15 | 33625892 | chr3 | 107318383 | chr11 | 1987961 |
| chr18 | 47786098 | chr16 | 87890498 | chr14 | 34393486 |
| chr12 | 16086133 | chr16 | 87661398 | chr17 | 39769175 |
| chr6 | 162040215 | chr3 | 128839536 | chrX | 49178526 |
| chr22 | 46344039 | chr16 | 3148824 | chr2 | 96051322 |
| chr12 | 11954080 | chr6 | 73330693 | chr22 | 47338827 |
| chr13 | 29233512 | chr22 | 28246158 | chr12 | 53298237 |
| chr5 | 132158216 | chr16 | 11768860 | chr11 | 11613810 |
| chr12 | 49934774 | chrX | 43832052 | chr12 | 119584195 |
| chr16 | 4049727 | chr13 | 92084253 | chr16 | 89387418 |
| chr12 | 117827187 | chr12 | 125028329 | chr14 | 100007924 |
| chr10 | 72576249 | chr16 | 4268336 | chr16 | 21448119 |
| chr3 | 49044952 | chr14 | 104614485 | chr19 | 10810503 |
| chr16 | 88837721 | chr19 | 7032457 | chr13 | 84455346 |
| chr12 | 52628285 | chr16 | 72047499 | chr2 | 23907893 |
| chr16 | 75105090 | chr13 | 27999126 | chr20 | 41200406 |
| chr20 | 13254989 | chr14 | 103864937 | chr20 | 24608264 |
| chr5 | 178287838 | chr13 | 111831177 | chr12 | 54425446 |
| chr18 | 74188818 | chr18 | 13912474 | chr17 | 15552895 |
| chr6 | 2902516 | chr17 | 48912710 | chr13 | 99687193 |
| chr17 | 46621439 | chr16 | 22330105 | chr20 | 40100677 |
| chr2 | 85638721 | chr14 | 95693981 | chr9 | 127703802 |
| chr16 | 25711473 | chr20 | 18693374 | chr14 | 91471236 |
| chr2 | 178078419 | chr19 | 36631360 | chr14 | 91855671 |
| chr9 | 101570745 | chr2 | 134023445 | chr11 | 122848577 |
| chr13 | 84454324 | chr3 | 93605923 | chr12 | 48187601 |
| chr20 | 39976385 | chr14 | 100204041 | chr13 | 21407476 |
| chr14 | 105881989 | chr3 | 57260956 | chr14 | 100573335 |
| chrX | 24071970 | chr5 | 139488638 | chr12 | 116641481 |
| chr6 | 166749267 | chr6 | 73356528 | chr3 | 63474931 |
| chr6 | 2891300 | chr14 | 20295845 | chr3 | 183945284 |
| chr13 | 111085649 | chr15 | 42360268 | chr9 | 115513981 |
| chr16 | 17322561 | chr14 | 68282983 | chr18 | 77199459 |
| chr19 | 33792464 | chr12 | 104070869 | chr3 | 64547323 |
| chr13 | 80914582 | chr6 | 101846905 | chr20 | 24639650 |
| chr12 | 125004095 | chr16 | 66505404 | chr15 | 80861601 |
| chr2 | 23611243 | chr14 | 75128828 | chr16 | 87725673 |
| chr12 | 54338819 | chr3 | 116382513 | chr7 | 111845429 |
| chr11 | 64646286 | chr15 | 99271049 | chr20 | 55975496 |
| chr10 | 7861257 | chr20 | 40172803 | chr15 | 90718504 |
| chr2 | 24582621 | chr15 | 74724686 | chr19 | 935267 |
| chr7 | 65584898 | chr15 | 54003232 | chr13 | 110936343 |
| chr12 | 96253464 | chr17 | 78079671 | chr20 | 24598154 |
| chr12 | 584675 | chr14 | 22958482 | chr20 | 41620914 |
| chr2 | 220094987 | chr15 | 23811043 | chr6 | 35778755 |
| chr11 | 131407861 | chr16 | 81898434 | chr11 | 96059902 |
| chrX | 107018530 | chr18 | 10740861 | chr11 | 61197848 |
| chr17 | 18941971 | chr11 | 65292000 | chr5 | 11632009 |
| chr18 | 74125626 | chr2 | 33668036 | chr19 | 14184884 |
| chr3 | 77331148 | chr12 | 62586655 | chr16 | 17199385 |
| chr7 | 150707235 | chr13 | 74425315 | chr17 | 72341014 |
| chr11 | 2859186 | chr20 | 45618201 | chr15 | 29504631 |
| chr10 | 49732876 | chr12 | 117771727 | chr12 | 1704443 |
| chr20 | 1103991 | chr19 | 46195136 | chr12 | 56116227 |
| chr20 | 20374037 | chr11 | 35641938 | chr16 | 4815786 |
| chr19 | 44764837 | chr13 | 50932095 | chr14 | 74607285 |
| chr5 | 38583054 | chr12 | 49719047 | chr12 | 109716912 |
| chr14 | 90863600 | chr12 | 53784323 | chr3 | 79815729 |
| chr15 | 75109505 | chr2 | 63278108 | chr14 | 24778911 |
| chr16 | 89574022 | chr15 | 79096751 | chr9 | 21973527 |
| chr2 | 77820399 | chr18 | 72917447 | chr17 | 46221813 |
| chr2 | 85553813 | chr14 | 74033301 | chr16 | 87893470 |
| chr15 | 40456724 | chr5 | 149867186 | chr11 | 68699932 |
| chr12 | 111332408 | chr14 | 77491876 | chr16 | 70722541 |
| chr5 | 169063590 | chr18 | 10463531 | chr19 | 2867898 |
| chr17 | 42966379 | chr13 | 111365899 | chr17 | 19551214 |
| chr5 | 179344751 | chr5 | 145583807 | chr2 | 220110584 |
| chr6 | 37244858 | chr19 | 48673525 | chr12 | 29430585 |
| chr19 | 18706598 | chr3 | 122514476 | chr15 | 65188459 |
| chr3 | 49029310 | chr12 | 54427310 | chr2 | 7118707 |
| chr20 | 61444138 | chr15 | 101877370 | chr18 | 25730190 |
| chr3 | 133659690 | chr11 | 44601199 | chr20 | 5765597 |
| chr2 | 176945893 | chr14 | 77669529 | chr15 | 68051838 |
| chr16 | 88812638 | chr2 | 239009050 | chr7 | 158888063 |
| chr14 | 58640305 | chr13 | 111108504 | chr12 | 108079458 |
| chr5 | 76935246 | chr18 | 12912293 | chr15 | 78930633 |
| chr8 | 143918218 | chr12 | 123753032 | chr2 | 9562567 |
| chr12 | 49257664 | chr16 | 70285978 | chr12 | 124847753 |
| chr17 | 72340380 | chr3 | 4822623 | chr15 | 101442826 |
| chr5 | 52918659 | chr12 | 124897560 | chr12 | 103285072 |
| chr11 | 68139098 | chr6 | 76060940 | chr11 | 68149325 |
| chr17 | 77909749 | chr11 | 69089564 | chr15 | 75190716 |
| chr11 | 89606016 | chr14 | 105529553 | chr3 | 143690246 |
| chr15 | 74365266 | chr20 | 55981467 | chr20 | 23028785 |
| chr2 | 172172747 | chr19 | 19248569 | chr6 | 90170712 |
| chr6 | 170892994 | chr22 | 47549830 | chr14 | 74807607 |
| chr2 | 46844498 | chr11 | 61451702 | chr6 | 76059734 |
| chr22 | 38477780 | chr9 | 24542255 | chr6 | 131285322 |
| chr12 | 105031680 | chr18 | 56531628 | chr12 | 2278951 |
| chr16 | 55478577 | chr5 | 78279882 | chr15 | 73631717 |
| chr11 | 2710838 | chr12 | 15275410 | chr19 | 4455413 |
| chr12 | 54350294 | chr12 | 85631372 | chr16 | 87698990 |
| chr6 | 142622584 | chr6 | 37894682 | chr15 | 85359523 |
| chr7 | 155599942 | chr17 | 73030211 | chr15 | 67417860 |
| chr17 | 48261653 | chr14 | 54863719 | chr12 | 69005196 |
| chr14 | 34864772 | chr22 | 28277628 | chr3 | 31900140 |
| chr11 | 70956646 | chr17 | 9804778 | chr13 | 110927758 |
| chr8 | 87881603 | chr3 | 184301518 | chr6 | 39787507 |
| chr3 | 50365476 | chr14 | 72857101 | chr11 | 44628644 |
| chr2 | 10538297 | chr14 | 52478367 | chr11 | 21386631 |
| chr15 | 75336956 | chr20 | 30034416 | chr11 | 68138850 |
| chr11 | 61486497 | chr18 | 77218474 | chr15 | 72511414 |
| chr5 | 905692 | chr5 | 138284831 | chr3 | 196467917 |
| chr6 | 74223642 | chr2 | 152894427 | chr20 | 10614679 |
| chr20 | 37274925 | chr22 | 37914655 | chr13 | 111298808 |
| chr7 | 122064307 | chr12 | 52301505 | chr12 | 95551716 |
| chr11 | 95976442 | chr14 | 20925263 | chr12 | 2268381 |
| chr9 | 21996690 | chr9 | 132460314 | chr17 | 6312011 |
| chr22 | 41866490 | chr15 | 67460700 | chr12 | 14060146 |
| chr5 | 177034561 | chr2 | 25139131 | chr19 | 18836407 |
| chr11 | 72525927 | chr20 | 37257596 | chr12 | 117884597 |
| chr14 | 99981191 | chr12 | 56211874 | chr14 | 69070944 |
| chr22 | 46326806 | chr15 | 79007816 | chr17 | 80225605 |
| chr16 | 88844415 | chr20 | 388144 | chr20 | 25746002 |
| chr9 | 139686019 | chr19 | 405313 | chr17 | 77764398 |
| chr14 | 92981723 | chr11 | 123045794 | chr5 | 171646468 |
| chr12 | 94543524 | chr6 | 148664420 | chr13 | 113758242 |
| chr11 | 189833 | chr9 | 21995197 | chr11 | 114050079 |
| chr15 | 41244632 | chr13 | 42032635 | chr15 | 40545511 |
| chr19 | 30889459 | chr6 | 78172277 | chr3 | 128744760 |
| chr3 | 98560883 | chr15 | 78942436 | chr5 | 40680446 |
| chr22 | 28186862 | chr9 | 135546700 | chr2 | 227661381 |
| chr5 | 7820781 | chr20 | 13204581 | chr20 | 6101929 |
| chr19 | 11529574 | chr9 | 139939347 | chr18 | 18606618 |
| chr17 | 72767008 | chr16 | 87889666 | chr3 | 79815007 |
| chr2 | 96801090 | chr12 | 122232323 | chr17 | 21477645 |
| chr13 | 39262227 | chr18 | 77245487 | chr20 | 57471122 |
| chr11 | 31846188 | chr5 | 99878914 | chr18 | 21769429 |
| chr14 | 90868250 | chr15 | 92407548 | chrX | 48547839 |
| chr9 | 126792233 | chr14 | 61110002 | chr11 | 61468526 |
| chr12 | 6450539 | chr12 | 25102196 | chr12 | 101787956 |
| chr3 | 58038562 | chr19 | 50193737 | chr11 | 61342332 |
| chr17 | 42336687 | chr6 | 40381264 | chr18 | 57101415 |
| chr18 | 25755897 | chr11 | 62455849 | chr13 | 36101743 |
| chr6 | 136335824 | chr11 | 72396829 | chr9 | 34724831 |
| chr20 | 35673037 | chr22 | 31556828 | chr13 | 111869203 |
| chr8 | 10131565 | chr11 | 6440614 | chr14 | 102742517 |
| chr19 | 13512786 | chr11 | 71949351 | chr5 | 172272430 |
| chr17 | 46226125 | chr13 | 96131459 | chr14 | 69070659 |
| chr12 | 51736360 | chr11 | 126188475 | chr20 | 1163295 |
| chr16 | 66550612 | chr6 | 17485659 | chr3 | 60316163 |
| chr20 | 36662866 | chr19 | 11435430 | chr22 | 29650483 |
| chr19 | 58095699 | chr12 | 104156552 | chr14 | 103513151 |
| chr19 | 7032549 | chr6 | 19839518 | chr19 | 48821028 |
| chr12 | 65672082 | chr5 | 75867305 | chr12 | 83248478 |
| chr12 | 11872884 | chr14 | 77600217 | chr15 | 51998416 |
| chr20 | 13255338 | chr12 | 58088019 | chr17 | 77991007 |
| chr5 | 6453988 | chr16 | 89470029 | chr17 | 54398065 |
| chr22 | 41214867 | chr16 | 31885529 | chr17 | 74897884 |
| chr19 | 1967352 | chr11 | 118793284 | chr13 | 99635091 |
| chr19 | 8190876 | chr17 | 40260704 | chr12 | 125017105 |
| chr12 | 117805057 | chr12 | 54388821 | chr18 | 47482543 |
| chr19 | 19367084 | chr2 | 42795242 | chr18 | 21512521 |
| chr11 | 27016530 | chr16 | 17563004 | chr4 | 789158 |
| chr4 | 174444205 | chr5 | 121297915 | chr20 | 31136845 |
| chr10 | 23633690 | chr12 | 117317704 | chr17 | 40018828 |
| chr7 | 99614007 | chr22 | 20302404 | chr9 | 6414308 |
| chr22 | 37536084 | chr11 | 65314498 | chr11 | 61341207 |
| chr12 | 62606860 | chr6 | 166744279 | chr20 | 36707837 |
| chr10 | 4973799 | chr13 | 108303589 | chr19 | 46269978 |
| chr3 | 21822610 | chr6 | 43483025 | chr12 | 124826078 |
| chr16 | 17469594 | chr22 | 43593595 | chr12 | 56137637 |
| chr14 | 68286569 | chr13 | 76174688 | chr3 | 196738111 |
| chr6 | 157978863 | chr20 | 8628780 | chr17 | 28804401 |
| chr6 | 96480082 | chr18 | 57220985 | chr2 | 212319644 |
| chr14 | 65512753 | chr3 | 15470158 | chr17 | 73876534 |
| chr22 | 29652183 | chr20 | 47589574 | chr11 | 69175088 |
| chr12 | 125023676 | chr17 | 29284048 | chr18 | 72175316 |
| chr20 | 31129482 | chr12 | 53343479 | chr12 | 11874044 |
| chr18 | 61088618 | chr18 | 70213394 | chr12 | 14114379 |
| chr17 | 7153738 | chr20 | 3139415 | chr5 | 180059067 |
| chr20 | 60791865 | chr3 | 178867159 | chr12 | 48171878 |
| chr12 | 4396004 | chr5 | 7851285 | chr11 | 120096499 |
| chr11 | 2800586 | chr20 | 633507 | chr20 | 54580489 |
| chr20 | 2539227 | chr14 | 70085866 | chr14 | 102719349 |
| chr3 | 126259929 | chr20 | 49163358 | chr15 | 52632412 |
| chr11 | 70478821 | chr20 | 55754230 | chr16 | 11768860 |
| chr2 | 54954699 | chr3 | 196595774 | chr13 | 107143980 |
| chr18 | 7047336 | chr14 | 104001122 | chr20 | 32255692 |
| chr11 | 111285431 | chr11 | 65559288 | chr14 | 69162068 |
| chr6 | 13356417 | chr22 | 37536737 | chr19 | 5059507 |
| chr13 | 27999126 | chr5 | 149430689 | chr16 | 2198097 |
| chr11 | 64761978 | chr2 | 235936653 | chr6 | 1393482 |
| chr19 | 37555642 | chr15 | 43976544 | chr14 | 74485639 |
| chr3 | 9872147 | chr18 | 44552959 | chr13 | 70358340 |
| chr6 | 24910750 | chr6 | 127836074 | chr3 | 175338932 |
| chr6 | 27834959 | chr6 | 41156642 | chr3 | 117631830 |
| chr22 | 38382245 | chr18 | 77905127 | chr19 | 1210522 |
| chr2 | 119603285 | chr11 | 35302805 | chr3 | 185225016 |
| chr19 | 46032329 | chr16 | 58718950 | chr5 | 59190048 |
| chr10 | 26504425 | chr14 | 105715553 | chr12 | 54666945 |
| chr17 | 32911834 | chr2 | 3828507 | chr20 | 48733402 |
| chr6 | 43213374 | chr11 | 114050079 | chr22 | 32955536 |
| chr15 | 65219093 | chr14 | 76178021 | chr14 | 61430527 |
| chr14 | 38073396 | chr17 | 73839630 | chr5 | 38846973 |
| chr13 | 47128881 | chr2 | 33701712 | chr20 | 62173763 |
| chr12 | 49939540 | chr11 | 73693059 | chr13 | 93962880 |

The predictive power of each CpG site may be ranked by the P-value of the individual sites in differentiating between non-CP and CP patients. Table 7 ranks the top 1000 sites by P-value.

TABLE 7

| Blood | Muscle | Satellite |
|---|---|---|
| CpG Site | CpG Site | CpG Site |
| chr3.0170984628 | chr16.0002184051 | chr22.0041683914 |
| chr12.0113912958 | chr17.0032964775 | chr3.0051997994 |
| chr5.0021333675 | chr20.0060998493 | chr11.0093971657 |
| chr1.0000565262 | chr17.0034497913 | chr20.0022553062 |
| chr18.0059002180 | chr13.0050656192 | chr20.0060375363 |
| chr22.0021469408 | chr9.0090747955 | chr12.0001742005 |
| chr16.0002689275 | chr17.0056596237 | chr19.0048673525 |
| chr12.0006446797 | chr15.0031516141 | chr16.0058069921 |
| chr15.0028343785 | chr17.0018941025 | chr18.0071801714 |
| chr6.0161033995 | chr6.0161034892 | chr15.0092982723 |
| chr18.0004068725 | chr11.0066189251 | chr16.0002176197 |
| chr12.0001704443 | chr12.0057869596 | chr11.0017593831 |
| chr11.0013984067 | chr16.0014782082 | chr4.0101719592 |
| chr2.0243078103 | chr18.0024130101 | chr14.0101270192 |
| chr19.0020188618 | chr16.0067564144 | chr13.0113650227 |
| chr6.0169653176 | chr14.0019641888 | chr19.0019007351 |
| chr6.0007394523 | chr6.0139013304 | chr18.0043923940 |
| chr6.0117587149 | chr14.0095048461 | chr12.0119584195 |
| chr12.0058160354 | chr16.0002184447 | chr2.0056193463 |
| chr13.0033220266 | chr14.0038061691 | chr12.0002513357 |
| chr12.0002113367 | chr6.0041528356 | chr13.0028891055 |
| chr13.0114547107 | chr17.0065040349 | chr22.0046279322 |
| chr18.0029214163 | chr6.0043445772 | chr11.0123045794 |
| chr11.0071160096 | chr17.0019045779 | chr5.0145724640 |
| chr3.0182928999 | chr6.0161062626 | chr9.0043084487 |
| chr6.0150284688 | chr6.0100901811 | chr15.0067989241 |
| chr11.0093475384 | chr13.0028196417 | chr15.0069824154 |
| chr16.0030886974 | chr16.0019728830 | chr12.0116068191 |
| chr17.0007975795 | chr14.0076447816 | chr12.0133301799 |
| chr6.0167001337 | chr16.0021504797 | chr18.0011747918 |
| chr22.0038022735 | chr12.0130645000 | chr16.0073089847 |
| chr12.0108079458 | chr20.0061696282 | chr16.0000680326 |
| chr11.0000188098 | chr9.0097811271 | chr17.0075149309 |
| chr3.0147130343 | chr6.0099969527 | chr14.0034393486 |
| chr13.0050656618 | chr12.0058088346 | chr11.0067044849 |
| chr3.0197687858 | chr3.0059803935 | chr12.0001609391 |
| chr6.0149354426 | chr12.0040909296 | chr15.0090964193 |
| chr6.0026235050 | chr6.0050811344 | chr20.0048793087 |
| chr17.0011145564 | chr15.0082977792 | chr17.0078426681 |
| chr9.0033025770 | chr11.0070664922 | chr12.0011513104 |
| chr12.0123009847 | chr12.0108040090 | chr5.0172124187 |
| chr15.0075932985 | chr11.0069257964 | chr20.0019221684 |
| chr11.0094276724 | chr14.0081040927 | chr20.0019447811 |
| chr18.0003771925 | chr9.0045729679 | chr20.0035217888 |
| chr7.0130353922 | chr2.0011879322 | chr2.0029850455 |
| chr3.0019188810 | chr3.0119422009 | chr18.0077209053 |
| chr18.0019674041 | chr17.0026166996 | chr20.0060893697 |
| chr12.0121082579 | chr14.0057284380 | chr13.0113705362 |
| chr13.0103425377 | chr12.0089410897 | chr2.0239051141 |
| chr12.0069005196 | chr11.0000796607 | chr14.0105643016 |
| chr6.0042418413 | chr5.0126641436 | chr22.0040899482 |
| chr17.0027482390 | chr18.0019064708 | chr14.0065204158 |
| chr2.0133110495 | chr12.0006437021 | chr13.0093896533 |
| chr12.0004758511 | chr16.0066864962 | chr3.0122711856 |
| chr22.0033197965 | chr5.0034839641 | chr18.0044547036 |
| chr19.0000920756 | chr11.0125774368 | chr16.0066879958 |
| chr17.0036286187 | chr3.0029361462 | chr12.0016644803 |
| chr5.0140782367 | chr18.0074700260 | chr11.0129750009 |
| chr6.0021594873 | chr12.0049484577 | chr19.0011533307 |
| chr20.0058801846 | chr20.0061703026 | chr3.0130802181 |
| chr13.0093896533 | chr6.0064283735 | chr18.0045011716 |
| chr16.0014818378 | chr16.0005573968 | chr12.0131689822 |
| chr16.0058000211 | chr14.0019400285 | chr16.0002004921 |
| chr19.0040723542 | chr20.0034287732 | chr3.0117631830 |
| chr2.0056193463 | chr8.0086756044 | chr22.0038022735 |
| chr12.0132855014 | chr6.0143859047 | chr22.0020779274 |
| chr16.0050582980 | chr19.0015580788 | chr12.0129065948 |
| chr12.0006663682 | chr22.0024816977 | chr3.0016939628 |
| chr20.0020258001 | chr14.0019902538 | chr12.0010243158 |
| chr15.0099344316 | chr11.0122933736 | chr12.0057608666 |
| chr17.0004850081 | chr14.0067984108 | chr15.0101588506 |
| chr20.0043513649 | chr13.0109856377 | chr12.0083436417 |
| chr13.0028497262 | chr17.0011145564 | chr18.0006284010 |
| chr20.0021689463 | chr20.0031330621 | chr14.0093019588 |
| chr20.0048919181 | chr5.0065124116 | chr2.0071533997 |
| chr22.0037977772 | chr3.0128719633 | chr16.0084054247 |
| chr3.0147128690 | chr18.0044556541 | chr4.0095555981 |
| chr15.0060302054 | chr3.0132136984 | chr3.0138666240 |
| chr15.0084114534 | chr6.0030640440 | chr2.0035092870 |
| chr10.0104179528 | chr12.0003207132 | chr20.0025001878 |
| chr16.0070557879 | chr16.0015239520 | chr6.0016145414 |
| chr3.0004508116 | chr18.0077559128 | chr16.0003238806 |
| chr11.0006633681 | chr15.0102331212 | chr12.0101447687 |
| chr20.0009100637 | chr12.0119596461 | chr5.0177181499 |
| chr19.0001238477 | chr14.0052266868 | chr14.0069861077 |
| chr10.0081665235 | chr14.0088459020 | chr12.0106978718 |
| chr13.0021069344 | chr19.0048836554 | chr12.0103344694 |
| chr12.0005019351 | chr2.0011878864 | chr9.0008638390 |
| chr6.0161067521 | chr2.0220379058 | chr6.0159600100 |
| chr6.0134210544 | chr5.0034182070 | chr19.0007038916 |
| chr18.0071642546 | chr11.0043605047 | chr17.0073069667 |
| chr18.0012287195 | chr14.0100626499 | chr17.0036292033 |
| chr12.0071439895 | chr5.0168726909 | chr5.0177219938 |
| chr14.0101308437 | chr3.0048884826 | chr14.0053155967 |
| chr3.0013541769 | chr11.0075162684 | chr15.0069452943 |
| chr3.0188420144 | chr14.0020089147 | chr2.0023747265 |
| chr15.0075072612 | chr6.0028831484 | chr17.0018941025 |
| chr6.0036737891 | chr8.0106331523 | chr5.0164550214 |
| chr14.0080170888 | chr6.0027776053 | chr17.0038475164 |
| chr14.0103575195 | chr19.0047733365 | chr5.0122422526 |
| chr17.0001390159 | chr16.0084212059 | chr3.0170886184 |
| chr6.0033280212 | chr2.0136874432 | chr14.0104190006 |
| chr14.0052119953 | chr12.0045006632 | chr12.0130645000 |
| chr3.0032022182 | chr12.0054376047 | chr20.0059927068 |
| chr8.0025900066 | chr20.0019419638 | chr6.0164766175 |
| chr18.0057357804 | chr16.0053738156 | chr14.0101909600 |
| chr20.0048552544 | chr6.0073879011 | chr14.0106437080 |
| chr12.0003241735 | chr14.0103984664 | chr17.0079292270 |
| chr13.0096206439 | chr16.0050746190 | chr14.0104465222 |
| chr17.0046675549 | chr16.0088271806 | chr19.0016830739 |
| chr17.0078944136 | chr3.0141746797 | chr11.0002405177 |
| chr16.0032773264 | chr15.0059668078 | chr15.0069516868 |
| chr6.0145853785 | chr10.0023479600 | chr17.0074566299 |
| chr13.0068862091 | chr5.0129242435 | chr9.0133777218 |
| chr18.0010938440 | chr20.0052660313 | chr2.0218771815 |
| chr13.0041796041 | chr16.0072883281 | chr12.0006104973 |
| chr2.0200819443 | chr15.0027604283 | chr3.0189596966 |
| chr11.0032113404 | chr5.0134094725 | chr15.0057921004 |
| chr18.0050319582 | chr15.0099707434 | chr14.0069738172 |
| chr19.0056616939 | chr3.0148581255 | chr20.0035145830 |
| chr20.0021083144 | chr6.0011065249 | chr17.0053343752 |
| chr14.0075389585 | chr12.0122601382 | chr9.0035610018 |
| chr9.0135118027 | chr20.0055212824 | chr17.0002701265 |
| chr16.0078675586 | chr22.0046477871 | chr5.0131564700 |
| chr18.0045934806 | chr5.0163762986 | chr2.0060196436 |
| chr16.0048639869 | chr17.0019046425 | chr14.0061281110 |
| chr22.0050166610 | chr18.0044774814 | chr18.0076738821 |
| chr6.0026159113 | chr14.0019422930 | chr22.0022129567 |
| chr20.0000633169 | chr17.0045177514 | chr6.0151563368 |
| chr5.0032712315 | chr17.0026989567 | chr3.0149299399 |
| chr20.0019192507 | chr13.0112761052 | chr13.0042220397 |
| chr17.0018992459 | chr15.0038545505 | chr2.0113399982 |
| chr12.0103980970 | chr15.0030515584 | chr15.0068639973 |
| chr3.0042740649 | chr15.0081426597 | chr19.0045735345 |
| chr17.0009939872 | chr5.0178194750 | chr16.0002478353 |
| chr15.0042565481 | chr9.0033044462 | chr14.0104416308 |
| chr12.0114348940 | chr2.0134918817 | chr17.0019045779 |
| chr2.0040678760 | chr20.0061428843 | chr17.0077893455 |
| chr18.0011986236 | chr6.0151562306 | chr20.0060885977 |
| chr9.0034335138 | chr11.0013690984 | chr13.0052817715 |
| chr22.0030821443 | chr6.0026597382 | chr11.0046135102 |
| chr5.0170869818 | chr19.0018366297 | chr15.0043244495 |
| chr1.0009123915 | chr14.0024802939 | chr13.0102572261 |
| chr16.0003239135 | chr20.0055800172 | chr3.0053784559 |
| chr16.0023706114 | chr5.0106822941 | chr11.0001945918 |
| chr13.0021141519 | chr5.0068938717 | chr16.0088038262 |
| chr14.0024457943 | chr6.0041528285 | chr19.0010249278 |
| chr12.0118540949 | chr13.0037575268 | chr21.0044484874 |
| chr18.0010483180 | chr6.0167765060 | chr17.0026101385 |
| chr11.0065688012 | chr5.0176766295 | chr14.0104977792 |
| chr17.0004852711 | chr15.0082758910 | chr20.0060501154 |
| chr22.0042739198 | chr22.0031709333 | chr12.0052817612 |
| chr6.0029717475 | chr16.0000745402 | chr6.0030746896 |
| chr2.0112826693 | chr22.0050747228 | chr15.0098986819 |
| chr12.0008662310 | chr16.0070207297 | chr15.0034647112 |
| chr13.0034116760 | chr3.0189791239 | chr5.0164483805 |
| chr12.0054409525 | chr15.0040014219 | chr12.0033043614 |
| chr6.0010695540 | chr3.0134370046 | chr16.0000312658 |
| chr13.0044679741 | chr14.0037124548 | chr21.0030689317 |
| chr22.0051060032 | chr17.0073390467 | chr10.0105420300 |
| chr15.0040397182 | chr20.0008703238 | chr14.0055966140 |
| chr9.0074764383 | chr15.0065386961 | chr14.0093530974 |
| chr20.0044659670 | chr10.0026501357 | chr14.0100040905 |
| chr3.0114172836 | chr6.0096464063 | chr17.0019046425 |
| chr16.0072910084 | chr6.0144729448 | chr14.0105065084 |
| chr12.0122097533 | chr5.0178594539 | chr2.0046561399 |
| chr18.0044542946 | chr18.0032609268 | chr14.0075517195 |
| chr16.0077953169 | chr20.0062184310 | chr15.0088659150 |
| chr12.0099834466 | chr16.0062003103 | chr12.0052981404 |
| chr11.0027384693 | chr6.0137864446 | chr22.0030084328 |
| chr6.0084056765 | chr11.0131618285 | chr15.0033445994 |
| chr14.0021268523 | chr22.0018788785 | chr15.0080573863 |
| chr16.0016127641 | chr9.0015423618 | chr12.0013354616 |
| chr9.0139609527 | chr13.0093879327 | chr17.0070499160 |
| chr14.0106351554 | chr5.0178728013 | chr9.0018605667 |
| chr20.0044639931 | chr11.0089722399 | chr11.0042088046 |
| chr14.0020111288 | chr2.0069171202 | chr20.0060272050 |
| chr17.0043361627 | chr22.0043627738 | chr15.0101482307 |
| chr14.0024906444 | chr16.0028621406 | chr10.0104354945 |
| chr3.0010056914 | chr6.0021730078 | chr14.0105040488 |
| chr6.0117997665 | chr16.0003173706 | chr12.0010242982 |
| chr3.0098482988 | chr5.0039219698 | chr11.0070369651 |
| chr3.0124866135 | chr14.0106200904 | chr18.0046461592 |
| chr11.0067204130 | chr17.0028705169 | chr12.0046149812 |
| chr6.0035420737 | chr17.0077180395 | chr22.0038010164 |
| chr18.0019760779 | chr15.0094881865 | chr2.0237117273 |
| chr3.0073232343 | chr17.0061778909 | chr3.0060920593 |
| chr6.0166078201 | chr15.0089688209 | chr17.0065622780 |
| chr13.0092454487 | chr14.0105010924 | chr8.0042999184 |
| chr3.0159563262 | chr14.0101014522 | chr9.0116266123 |
| chr11.0066312805 | chr6.0136872990 | chr12.0120463149 |
| chr15.0077713456 | chr5.0142054203 | chr12.0122395466 |
| chr14.0064687513 | chr22.0018676764 | chr9.0138653925 |
| chr11.0013512050 | chr22.0040395218 | chr16.0000555577 |
| chr5.0115178530 | chr2.0215672454 | chr13.0113678380 |
| chr14.0024800986 | chr12.0122598695 | chr12.0063544384 |
| chr8.0068877277 | chr15.0050280046 | chr3.0077077670 |
| chr12.0130529772 | chr6.0027270299 | chr6.0149608178 |
| chr11.0132589040 | chr6.0046293418 | chr22.0020244288 |
| chr13.0028529289 | chr20.0032407915 | chr13.0033220266 |
| chr14.0101291544 | chr13.0040919260 | chr16.0077912976 |
| chr3.0112013079 | chr11.0012000965 | chr6.0021679242 |
| chr6.0007909642 | chr16.0077912976 | chr14.0096729260 |
| chr13.0100648209 | chr3.0196750460 | chr12.0053189775 |
| chr19.0042037321 | chr14.0023826830 | chr12.0032410070 |
| chr22.0018990731 | chr19.0046366124 | chr2.0047454097 |
| chr14.0096893304 | chr14.0023696147 | chr13.0074262821 |
| chr13.0094830546 | chr5.0066462646 | chr18.0020972062 |
| chr11.0117688942 | chr18.0019179732 | chr3.0111469507 |
| chr11.0016626136 | chr8.0145539988 | chr12.0066791524 |
| chr11.0000829825 | chr15.0061212366 | chr15.0088174247 |
| chr18.0024131895 | chr13.0096137813 | chr5.0159719484 |
| chr12.0006677428 | chr16.0000810757 | chr3.0052260373 |
| chr11.0101982711 | chr18.0040184017 | chr3.0167620956 |
| chr1.0143743801 | chr14.0095140483 | chr8.0001828441 |
| chr6.0127836074 | chr16.0015950869 | chr10.0126136854 |
| chr5.0176899611 | chr18.0077918405 | chr14.0075019216 |
| chr3.0050230579 | chr12.0057039757 | chr12.0130099338 |
| chr14.0103396312 | chr16.0084404119 | chr11.0001418756 |
| chr14.0071108090 | chr6.0003849381 | chr17.0031110221 |
| chr14.0050236376 | chr19.0001787965 | chr22.0019946864 |
| chr14.0050064662 | chr17.0036600772 | chr13.0113087882 |
| chr16.0048200367 | chr20.0007905990 | chr14.0050108699 |
| chr15.0021071194 | chr9.0130829697 | chr16.0083715823 |
| chr9.0023825088 | chr12.0094543824 | chr15.0029644298 |
| chr6.0039174327 | chr19.0046520118 | chr15.0101984558 |
| chr15.0022052629 | chr11.0132584655 | chr14.0094578378 |
| chr16.0021610035 | chr13.0078562773 | chr17.0000112167 |
| chr6.0029975416 | chr5.0149989951 | chr20.0052825772 |
| chr6.0053518960 | chr11.0019739264 | chr6.0044243576 |
| chr3.0004508091 | chr22.0025448214 | chr10.0068984115 |
| chr20.0055965866 | chr16.0002175229 | chr5.0178687043 |
| chr17.0040610021 | chr17.0031110221 | chr17.0026875323 |
| chr3.0006946725 | chr20.0002739975 | chr3.0016419520 |
| chr12.0005992325 | chr18.0020303365 | chr11.0062211039 |
| chr7.0155248019 | chr12.0053216725 | chr22.0033976935 |
| chr16.0002176197 | chr5.0168279190 | chr22.0048977442 |
| chr6.0031913250 | chr6.0161063597 | chr12.0123780584 |
| chr12.0005019004 | chr20.0061697965 | chr22.0031890427 |
| chr12.0116710705 | chr12.0053732109 | chr11.0017784809 |
| chr14.0094441286 | chr13.0036044855 | chr2.0239196855 |
| chr6.0151710123 | chr5.0149669433 | chr14.0020112999 |
| chr6.0161066486 | chr3.0126136572 | chr12.0005898164 |
| chr14.0042075406 | chr20.0030030504 | chr11.0065066399 |
| chr12.0108718730 | chr18.0077634030 | chr22.0029704946 |
| chr2.0208182399 | chr13.0096199517 | chr5.0087955859 |
| chr22.0042914550 | chr13.0114825688 | chr22.0046472984 |
| chr12.0096337323 | chr15.0039873937 | chr5.0092767677 |
| chr18.0077552544 | chr12.0123591053 | chr22.0033739403 |
| chr6.0037666959 | chr1.0000565262 | chr14.0089771498 |
| chr13.0028196417 | chr15.0101866025 | chr2.0182549408 |
| chr18.0013895717 | chr11.0057308986 | chr11.0122674083 |
| chr7.0150717629 | chr2.0105484635 | chr6.0154640863 |
| chr14.0076981142 | chr12.0000089539 | chr12.0131588481 |
| chr18.0013451043 | chr10.0050857637 | chr14.0105034337 |
| chr12.0120676188 | chr2.0240270414 | chr3.0045798106 |
| chr6.0108879347 | chr16.0022220811 | chr2.0020252403 |
| chr6.0149141140 | chr11.0007563239 | chr13.0020646254 |
| chr17.0033816012 | chr16.0018444991 | chr16.0031091597 |
| chr1.0011552103 | chr16.0070156896 | chr12.0057602992 |
| chr17.0072958021 | chr11.0118937771 | chr3.0054664460 |
| chr11.0063769700 | chr3.0088198086 | chr2.0114025250 |
| chr16.0053078278 | chr3.0050712994 | chr16.0058780937 |
| chr20.0020434395 | chr6.0027114418 | chr19.0009956949 |
| chr16.0029481117 | chr14.0105360814 | chr4.0062951852 |
| chr9.0021967489 | chr11.0010388190 | chr20.0015883372 |
| chr18.0077267918 | chr13.0112983416 | chr12.0081306216 |
| chr20.0030511960 | chr16.0088276659 | chr13.0036668831 |
| chr3.0110791724 | chr17.0040826322 | chr11.0002408146 |
| chr12.0121613310 | chr11.0118402547 | chr17.0043201589 |
| chr3.0149531758 | chr16.0087893023 | chr3.0160006045 |
| chr6.0026596052 | chr20.0062150177 | chr13.0113224488 |
| chr12.0111625057 | chr15.0020689934 | chr18.0011979862 |
| chr12.0081761865 | chr18.0049902983 | chr17.0047518498 |
| chr6.0169651645 | chr20.0062134308 | chr11.0000766885 |
| chr3.0052266153 | chr20.0003022992 | chr15.0057680681 |
| chr14.0104207861 | chr18.0045934806 | chr15.0081187412 |
| chr3.0128879185 | chr17.0046648207 | chr16.0087893023 |
| chr6.0137112686 | chr14.0103987474 | chr12.0039057639 |
| chr15.0075081115 | chr2.0101736025 | chr2.0221113349 |
| chr17.0040714177 | chr20.0004705306 | chr12.0117176320 |
| chr12.0019398518 | chr8.0042356881 | chr11.0047295306 |
| chr12.0060488444 | chr2.0045240007 | chr20.0031275087 |
| chr3.0194407700 | chr11.0133821966 | chr16.0007479261 |
| chr3.0008809238 | chr13.0111013147 | chr11.0003638448 |
| chr19.0030030719 | chr3.0123358380 | chr2.0064955687 |
| chr11.0065486827 | chr2.0242147702 | chr11.0017601401 |
| chr19.0039832370 | chr18.0077166042 | chr14.0073050565 |
| chr2.0037424300 | chr2.0237479179 | chr20.0030285535 |
| chr14.0093125680 | chr11.0120755680 | chr16.0070746421 |
| chr12.0005992812 | chr15.0078076500 | chr17.0028803808 |
| chr18.0077289073 | chr3.0191068667 | chr11.0116705996 |
| chr14.0067955473 | chr20.0052825772 | chr19.0052801607 |
| chr6.0031619857 | chr13.0099918251 | chr22.0033791071 |
| chr3.0112643572 | chr13.0020139644 | chr20.0031490465 |
| chr12.0006445005 | chr15.0089422286 | chr11.0002800586 |
| chr22.0050182413 | chr15.0068634575 | chr11.0070397632 |
| chr14.0090422160 | chr17.0038138125 | chr19.0041126191 |
| chr20.0060005419 | chr3.0021787630 | chr18.0053091201 |
| chr12.0021534654 | chr22.0031608388 | chr11.0032197363 |
| chr12.0012600467 | chr15.0072667639 | chr15.0088159831 |
| chr16.0053538293 | chr22.0046496794 | chr10.0017068488 |
| chr3.0183945284 | chr13.0027998115 | chr17.0076513353 |
| chr20.0060111182 | chr6.0010842909 | chr17.0046667531 |
| chr20.0052227442 | chr12.0125550620 | chr17.0079501119 |
| chr12.0013994189 | chr15.0079101067 | chr12.0104187430 |
| chr11.0068476691 | chr17.0078079671 | chr13.0115035743 |
| chr12.0099139571 | chr20.0037899756 | chr9.0000947870 |
| chr17.0005461043 | chr15.0089702329 | chr5.0011535023 |
| chr7.0129422674 | chr5.0074245192 | chr15.0083088304 |
| chr6.0160402911 | chr15.0036327669 | chr15.0031423000 |
| chr18.0021768319 | chr11.0126244675 | chr18.0030519743 |
| chr14.0091095921 | chr2.0060984163 | chr6.0007765009 |
| chr20.0005081567 | chr20.0060852455 | chr12.0066354814 |
| chr12.0098909208 | chr17.0067577554 | chr20.0043344103 |
| chr13.0058207163 | chr2.0240250488 | chr22.0018433398 |
| chr20.0044934168 | chr16.0088272354 | chr2.0111791669 |
| chr17.0048691369 | chr6.0161046961 | chr14.0077057981 |
| chr13.0048583626 | chr14.0105348061 | chr14.0074764684 |
| chr11.0124712584 | chr6.0000617660 | chr20.0060431447 |
| chr17.0036719559 | chr2.0171628234 | chr2.0185490688 |
| chr1.0028319047 | chr12.0116068191 | chr16.0062003103 |
| chr11.0071951989 | chr16.0055434128 | chr5.0111578627 |
| chr6.0071277683 | chr16.0089976937 | chr6.0001768121 |
| chr2.0056193796 | chr16.0088555614 | chr16.0048419832 |
| chr20.0002645380 | chr13.0074425315 | chr16.0071043770 |
| chr11.0017553236 | chr3.0123553694 | chr3.0005182629 |
| chr12.0119419907 | chr14.0100233662 | chr6.0075912800 |
| chr13.0021347444 | chr22.0046980797 | chr15.0043969272 |
| chr19.0054711367 | chr20.0058663197 | chr15.0051977900 |
| chr15.0088218254 | chr18.0059416446 | chr15.0082092159 |
| chr12.0069345802 | chr18.0045862942 | chr15.0061121482 |
| chr12.0082743539 | chr12.0127227116 | chr12.0132130323 |
| chr15.0032905739 | chr5.0010253159 | chr9.0084208113 |
| chr22.0036751194 | chr12.0054377387 | chr6.0004100037 |
| chr20.0051154032 | chr3.0149769390 | chr15.0051280672 |
| chr16.0002536329 | chr22.0024315084 | chr15.0071044501 |
| chr11.0128595279 | chr12.0117518197 | chr12.0132249669 |
| chr20.0061584832 | chr5.0126626085 | chrX.0023418321 |
| chr2.0241496921 | chr7.0074617497 | chr22.0038696824 |
| chr6.0166359839 | chr2.0238647519 | chr14.0050108792 |
| chr5.0175958110 | chr17.0042392494 | chr15.0052549111 |
| chr14.0020256530 | chr19.0014474657 | chr9.0000286627 |
| chr14.0096136382 | chr6.0044151584 | chr13.0109856377 |
| chr9.0003181598 | chr6.0078172784 | chr20.0018295860 |
| chr22.0042737369 | chr9.0136036533 | chr18.0074616984 |
| chr2.0068290320 | chr9.0021995197 | chr15.0078008856 |
| chr14.0094503085 | chr12.0085672520 | chr5.0011632009 |
| chr8.0080740825 | chr16.0027871719 | chr8.0001112153 |
| chr3.0010399607 | chr6.0016145414 | chr6.0071209322 |
| chr16.0075134924 | chr11.0061532358 | chr12.0001241667 |
| chr5.0149524459 | chr9.0042847832 | chr12.0023768031 |
| chr17.0034497703 | chr18.0040236519 | chr12.0003242436 |
| chr11.0068672559 | chr12.0003404344 | chr13.0047191668 |
| chr9.0119807716 | chr18.0074826797 | chr14.0094215266 |
| chr16.0031226193 | chr12.0106696764 | chr14.0034616607 |
| chr14.0093500776 | chr16.0033217069 | chr3.0009152200 |
| chr16.0030104676 | chr2.0223550141 | chr22.0046561026 |
| chr20.0011214507 | chr6.0041516396 | chr12.0128790228 |
| chr3.0134325344 | chr16.0062065715 | chr5.0080565262 |
| chr3.0181419991 | chr11.0118230644 | chr5.0149438902 |
| chr11.0123448701 | chr11.0002430664 | chr14.0047624779 |
| chr18.0043419102 | chr12.0122520621 | chr6.0100903182 |
| chr6.0028863896 | chr13.0100548878 | chr18.0042403544 |
| chr20.0036661494 | chr15.0020681909 | chr2.0010545065 |
| chr14.0093555378 | chr6.0043582726 | chr18.0024443615 |
| chr14.0029224940 | chr16.0000970346 | chr17.0011390406 |
| chr2.0015870812 | chr15.0093351469 | chr11.0092261532 |
| chr20.0033159135 | chr5.0168363687 | chr15.0039617456 |
| chr2.0038978853 | chr2.0054894075 | chr12.0126468360 |
| chr22.0042832848 | chr15.0096873280 | chr2.0038389310 |
| chr11.0001776846 | chr22.0037416860 | chr11.0002911200 |
| chr2.0008520249 | chr5.0131705769 | chr6.0044242577 |
| chr12.0051441921 | chr20.0051777029 | chrX.0109931974 |
| chr11.0068767987 | chr2.0201560149 | chr16.0082973314 |
| chr6.0132816345 | chr9.0042267178 | chr21.0046032860 |
| chr6.0119381919 | chr8.0001600120 | chr12.0067447796 |
| chr2.0045185087 | chr5.0122422526 | chr12.0000313066 |
| chr13.0050194640 | chr12.0125814703 | chr5.0011534641 |
| chr5.0140581653 | chr20.0057190376 | chr22.0019036590 |
| chr3.0042262164 | chr12.0122609637 | chr20.0045860407 |
| chr18.0050269807 | chr17.0075205588 | chr12.0053958599 |
| chr3.0183536414 | chr14.0105619424 | chr13.0100228957 |
| chr17.0080272432 | chr11.0073693837 | chr22.0029511825 |
| chr11.0002935626 | chr20.0034042530 | chr18.0018606618 |
| chr13.0099737576 | chr14.0101007959 | chr14.0020922933 |
| chr11.0119193563 | chr11.0064037947 | chr2.0023747257 |
| chr11.0122932346 | chr15.0074472301 | chr12.0095551716 |
| chr12.0050795886 | chr17.0076920331 | chr15.0042067504 |
| chr22.0030476452 | chr12.0124951222 | chr20.0019488775 |
| chr12.0113230085 | chr22.0039924996 | chr11.0009068783 |
| chr17.0004440254 | chr12.0054375682 | chr7.0140039833 |
| chr11.0118937771 | chr12.0110011702 | chr20.0019419638 |
| chr2.0095691796 | chr6.0041516444 | chr20.0025513653 |
| chr6.0096840641 | chr6.0044355950 | chr20.0060375820 |
| chr2.0074777409 | chr9.0112919418 | chr15.0079070483 |
| chr3.0042653997 | chr6.0064254064 | chr16.0070734806 |
| chr7.0002860289 | chr22.0040394914 | chr20.0036854808 |
| chr17.0079800852 | chr5.0021554765 | chr18.0040184017 |
| chr15.0093158225 | chr6.0000711630 | chr7.0075004051 |
| chr7.0144098652 | chr16.0018430894 | chr3.0071153921 |
| chr3.0045837192 | chr11.0118230307 | chr12.0005678978 |
| chr16.0021436223 | chr20.0060111182 | chr12.0110563292 |
| chr3.0021762167 | chr20.0018036354 | chr14.0051538766 |
| chr14.0024584488 | chr12.0027489742 | chr17.0073837683 |
| chr11.0033217978 | chr20.0061730889 | chr7.0074333014 |
| chr3.0138066730 | chr17.0018221878 | chr15.0028343785 |
| chr14.0054414208 | chr4.0077870028 | chr11.0064012692 |
| chr11.0043598653 | chr12.0053401055 | chr11.0067049045 |
| chr14.0037289739 | chr9.0130615047 | chr14.0027319786 |
| chr12.0131027301 | chr17.0076920337 | chr3.0023957967 |
| chr14.0034421550 | chr15.0102504167 | chr7.0138661012 |
| chr22.0039417803 | chr5.0169997723 | chr15.0032004823 |
| chr3.0073929061 | chr11.0032457407 | chr17.0042636770 |
| chr8.0067342312 | chr15.0099271049 | chr12.0000650002 |
| chr6.0167786484 | chr12.0003239960 | chr20.0017403368 |
| chr11.0020784643 | chr16.0086612198 | chr16.0004815786 |
| chr20.0055041683 | chr8.0022457514 | chr5.0083244775 |
| chr18.0076933945 | chr20.0031357187 | chr14.0095999996 |
| chr14.0020070761 | chr16.0075590275 | chr16.0001334537 |
| chr15.0028953236 | chr2.0145278174 | chr22.0025563508 |
| chr16.0032120111 | chr14.0093516554 | chr17.0064774558 |
| chr11.0059823213 | chr16.0075183152 | chr19.0049524228 |
| chr14.0101923185 | chr20.0022567432 | chr17.0034406350 |
| chr13.0112268133 | chr14.0094578378 | chr18.0046475320 |
| chr16.0086529283 | chr18.0019746697 | chr20.0047278938 |
| chr11.0032112972 | chr20.0003993509 | chr11.0114132025 |
| chr15.0075275818 | chr9.0131041994 | chr11.0035250260 |
| chr15.0052224564 | chr12.0006104973 | chr15.0069894932 |
| chr20.0062735385 | chr17.0015173307 | chr20.0037554365 |
| chr14.0051342013 | chr6.0042009656 | chr13.0030932893 |
| chr11.0125201806 | chr17.0078893711 | chr3.0139013512 |
| chr11.0131170372 | chr12.0065672082 | chr20.0044107407 |
| chr14.0020268631 | chr16.0087725673 | chr17.0049983647 |
| chr22.0025798718 | chr15.0102506257 | chr12.0002398915 |
| chr16.0015777363 | chr5.0017402448 | chr12.0066742963 |
| chr20.0061798378 | chr20.0030110087 | chr14.0057436305 |
| chr11.0102448364 | chr6.0161916305 | chr11.0120972213 |
| chr3.0059758690 | chr7.0045150844 | chr12.0102034085 |
| chr22.0031709333 | chr6.0000776290 | chr19.0050184400 |
| chr22.0023524799 | chr13.0019984659 | chr13.0095256511 |
| chr17.0037896074 | chr12.0124849639 | chr2.0001598523 |
| chr6.0073676215 | chr17.0080823228 | chr6.0111244762 |
| chr6.0029716615 | chr20.0061447334 | chr17.0078441727 |
| chr18.0000904579 | chr14.0065400868 | chr12.0039484618 |
| chr11.0063246601 | chr3.0071354642 | chr2.0207422413 |
| chr12.0089410897 | chr20.0060741351 | chr11.0112152163 |
| chr3.0033709568 | chr20.0060934620 | chr3.0132565636 |
| chr3.0185928189 | chr16.0000126451 | chr9.0075202282 |
| chr3.0012883119 | chr5.0148642943 | chr22.0038573766 |
| chr15.0022386873 | chr15.0099414436 | chr14.0089833851 |
| chr22.0018810468 | chr16.0021512098 | chr12.0120234027 |
| chr20.0062096425 | chr11.0006588675 | chr5.0128430539 |
| chr16.0023520796 | chr12.0054368501 | chr18.0021691205 |
| chr6.0097047563 | chr14.0088107483 | chr13.0047010408 |
| chr11.0064069091 | chr9.0015306304 | chr22.0046504129 |
| chr17.0079380897 | chr14.0070690454 | chr3.0053806824 |
| chr11.0116705996 | chr8.0092053718 | chr12.0112591200 |
| chr9.0134056175 | chr5.0131595932 | chr18.0043301302 |
| chr20.0031253292 | chr11.0059058735 | chr3.0047630484 |
| chr17.0079383185 | chr22.0024307433 | chr12.0122768292 |
| chr6.0167164659 | chr12.0121092873 | chr13.0114825688 |
| chr3.0185653023 | chr16.0067867925 | chr20.0040847575 |
| chr3.0004535738 | chr20.0061995332 | chr20.0062445219 |
| chr6.0089970486 | chr16.0084403863 | chr16.0078800215 |
| chr12.0031123933 | chr15.0020751824 | chr5.0106822941 |
| chr11.0064643975 | chr1.0000566293 | chr13.0019443315 |
| chr22.0021399663 | chr3.0178866452 | chr17.0031370105 |
| chr14.0037667459 | chr12.0053512857 | chr8.0086566777 |
| chr16.0050060741 | chr14.0102785973 | chr22.0026867489 |
| chr17.0043367233 | chr13.0114802848 | chr15.0089869670 |
| chr6.0028751116 | chr11.0062648042 | chr11.0076392234 |
| chr9.0129937134 | chr11.0046911477 | chr2.0223550141 |
| chr11.0128341522 | chr3.0025007031 | chr22.0018376899 |
| chr17.0007341212 | chr13.0067319615 | chr3.0108773581 |
| chr5.0139070243 | chr19.0006067391 | chr20.0036843383 |
| chr20.0045946429 | chr17.0055123996 | chr11.0099244166 |
| chr15.0085176255 | chr14.0020925176 | chr16.0057821860 |
| chr16.0000723354 | chr5.0151152326 | chr10.0080833721 |
| chr2.0086941621 | chr14.0095981206 | chr20.0048603774 |
| chr11.0131701732 | chr18.0034912967 | chr12.0054043247 |
| chr14.0091866859 | chr11.0011995234 | chr15.0068885184 |
| chr3.0184081196 | chr2.0010260899 | chr15.0079292108 |
| chr8.0027206271 | chr7.0002562236 | chr9.0138974172 |
| chr3.0014692909 | chr6.0077272083 | chr1.0032307779 |
| chr3.0013592045 | chr14.0102183435 | chr12.0025101805 |
| chr3.0113344967 | chr13.0098959659 | chr20.0043391328 |
| chr3.0157813756 | chr22.0020410726 | chr20.0036859462 |
| chr22.0019752899 | chr12.0122587932 | chr14.0020089147 |
| chr16.0067841171 | chr5.0141489233 | chr2.0010200647 |
| chr14.0050086676 | chr15.0023468448 | chr2.0209313215 |
| chr11.0106679557 | chr11.0125773076 | chr17.0034580968 |
| chr8.0019960971 | chr17.0036291754 | chr20.0060928261 |
| chr20.0021093596 | chr11.0000377521 | chr16.0011058849 |
| chr6.0031831095 | chr18.0055106886 | chr13.0107176083 |
| chr11.0060692513 | chr20.0052769132 | chr12.0015612205 |
| chr12.0114847931 | chr14.0105309704 | chr15.0050873417 |
| chr19.0041025562 | chr13.0028559519 | chr14.0094629010 |
| chr16.0034490054 | chr3.0032280657 | chr7.0044807421 |
| chr9.0099618153 | chr15.0097966805 | chr12.0124860589 |
| chr11.0067034843 | chr13.0032421265 | chr12.0130090963 |
| chr13.0112724188 | chr20.0047243056 | chr14.0061919248 |
| chr14.0074222043 | chr11.0125773069 | chr12.0052779236 |
| chr6.0137864446 | chr3.0010383658 | chr11.0000597676 |
| chr7.0089783680 | chr18.0034926192 | chr13.0100623759 |
| chr16.0057662526 | chr12.0113520131 | chr11.0063901203 |
| chr22.0029840112 | chr17.0002807629 | chr9.0079793512 |
| chr6.0164007026 | chr16.0071417927 | chr17.0065727540 |
| chr12.0127421303 | chr11.0110078840 | chr3.0099514929 |
| chr11.0129936009 | chr13.0042614083 | chr15.0078076885 |
| chr14.0093074566 | chr16.0028550298 | chr13.0033765016 |
| chr9.0023840705 | chr16.0056445309 | chr3.0178199704 |
| chr3.0195633765 | chr9.0073889367 | chr19.0009468870 |
| chr11.0019533325 | chr6.0087865978 | chr11.0070384416 |
| chr2.0153450964 | chr13.0029247722 | chr15.0040545511 |
| chr11.0121431954 | chr22.0037421734 | chr16.0029168316 |
| chr6.0145812428 | chr16.0009058197 | chr12.0109897832 |
| chr12.0065038369 | chr3.0133786270 | chr14.0039235379 |
| chr17.0034496197 | chr6.0073866782 | chr3.0125867487 |
| chr6.0145865884 | chr3.0011624243 | chr3.0056912875 |
| chr3.0014339294 | chr13.0113683538 | chr11.0115343899 |
| chr11.0075139544 | chr16.0078812736 | chr3.0196595774 |
| chr18.0046277091 | chr2.0070251589 | chr17.0080152801 |
| chr2.0063279693 | chr20.0061694983 | chr11.0046483909 |
| chr3.0039449150 | chr9.0131123686 | chr12.0056810802 |
| chr17.0075081745 | chr15.0030119730 | chr3.0180905890 |
| chr16.0015188855 | chr20.0061697330 | chr14.0105753103 |
| chr3.0042646477 | chr6.0163235590 | chr17.0011356330 |
| chr13.0028491525 | chr16.0029826679 | chr3.0037374195 |
| chr6.0123111308 | chr22.0018507076 | chr12.0113335076 |
| chr3.0012354426 | chr16.0002561757 | chr14.0081456317 |
| chr13.0032663317 | chr19.0001827105 | chr22.0049199174 |
| chr3.0010334779 | chr18.0039534866 | chr20.0035201174 |
| chr19.0036352376 | chr14.0103961164 | chr13.0102752821 |
| chr14.0047611368 | chr11.0070664902 | chr2.0102423165 |
| chr12.0131959552 | chr15.0023811043 | chr12.0095457527 |
| chr20.0043161260 | chr12.0124810206 | chr8.0140921136 |
| chr12.0109026041 | chr2.0106005147 | chr2.0235873378 |
| chr3.0004874239 | chr20.0025834932 | chr13.0036606864 |
| chr22.0016174941 | chr5.0098105942 | chr2.0069171202 |
| chr3.0134370046 | chr2.0129027501 | chr22.0028173173 |
| chr3.0141595150 | chr19.0003374991 | chr5.0032690664 |
| chr11.0093063959 | chr9.0126163665 | chr20.0058775716 |
| chr3.0097645615 | chr17.0026634443 | chr13.0096130928 |
| chr20.0002827499 | chr14.0077591807 | chr13.0079040535 |
| chr22.0023568235 | chr16.0062059434 | chr5.0057845264 |
| chr12.0070133619 | chr2.0103400490 | chr15.0066646204 |
| chr15.0063891971 | chr2.0085839678 | chr20.0003316125 |
| chr17.0056409820 | chr2.0001724761 | chr13.0063769342 |
| chr5.0150762866 | chr2.0242665083 | chr6.0032026797 |
| chr16.0021425800 | chr3.0022008166 | chr20.0034214499 |
| chr16.0033840313 | chr12.0100306357 | chr11.0031087495 |
| chr6.0006320434 | chr12.0014798861 | chr14.0052593986 |
| chr11.0085341829 | chr2.0171574558 | chr17.0077908058 |
| chr11.0067767580 | chr15.0071040854 | chr15.0031451635 |
| chr8.0011415889 | chr5.0014778676 | chr3.0148729673 |
| chr15.0102466363 | chr14.0105633102 | chr12.0123942691 |
| chr5.0174907220 | chr13.0108180002 | chr19.0014089299 |
| chr11.0046260296 | chr11.0000766885 | chr11.0094428046 |
| chr13.0048894526 | chr12.0121089092 | chr9.0136600382 |
| chr14.0021387252 | chr2.0105474450 | chr9.0097322146 |
| chr11.0032455081 | chr10.0073769075 | chr14.0100588329 |
| chr6.0113952412 | chr12.0054387478 | chr20.0060933723 |
| chr19.0050179634 | chr17.0040559108 | chr7.0005517660 |
| chr3.0184278878 | chr11.0085511840 | chr18.0045776354 |
| chr11.0000168146 | chr17.0039093719 | chr11.0132617559 |
| chr15.0020777505 | chr12.0129612373 | chr3.0004241375 |
| chr16.0071011081 | chr12.0132258996 | chr15.0041023492 |
| chr11.0047170869 | chr3.0189838858 | chr15.0034108303 |
| chr3.0196946735 | chr14.0064838686 | chr12.0107831478 |
| chr9.0125674963 | chr17.0036346142 | chr6.0166347327 |
| chr12.0066276259 | chr11.0041687132 | chr17.0008219019 |
| chr11.0102659645 | chr22.0025676078 | chr15.0065043833 |
| chr3.0108203622 | chr18.0019063659 | chr12.0049484577 |
| chr14.0094425060 | chr8.0099130384 | chr19.0002867898 |
| chr14.0060976921 | chr15.0054531058 | chr12.0026672531 |
| chr3.0048700361 | chr6.0015265499 | chr13.0041752980 |
| chr12.0021590843 | chr20.0060723991 | chr12.0122984696 |
| chr18.0039599079 | chr11.0069175088 | chr20.0023859143 |
| chr14.0095721522 | chr13.0025502416 | chr2.0020768167 |
| chr18.0014129770 | chr5.0126641880 | chr14.0103649648 |
| chr18.0005288345 | chr15.0077981635 | chr9.0139395217 |
| chr17.0048619793 | chr14.0101137458 | chr20.0062522605 |
| chr20.0058802294 | chr20.0025254024 | chr20.0062589386 |
| chr3.0035719313 | chr10.0043601430 | chr5.0171756702 |
| chr20.0010656610 | chr15.0066999962 | chr5.0058092517 |
| chr3.0194116885 | chr6.0148032878 | chr3.0055523173 |
| chr12.0080139936 | chr6.0028558113 | chr22.0044562387 |
| chr16.0083167195 | chr16.0070937543 | chr6.0168843099 |
| chr12.0054774703 | chr19.0039903634 | chr20.0062786731 |
| chr16.0029350163 | chr11.0124954687 | chr20.0061284334 |
| chr16.0027904366 | chr18.0074827147 | chr19.0015108434 |
| chr20.0040848410 | chr18.0013309358 | chr20.0062259792 |
| chr11.0118965966 | chr20.0062173702 | chr15.0069854566 |
| chr16.0089785669 | chr11.0108829727 | chr13.0099841980 |
| chr16.0003184592 | chr8.0022960878 | chr15.0027786719 |
| chr17.0002718386 | chr14.0061706209 | chr12.0001858063 |
| chr20.0061087268 | chr12.0121177060 | chr3.0182987901 |
| chr5.0131238304 | chr9.0096087601 | chr6.0168625864 |
| chr6.0112536337 | chr22.0039634447 | chr15.0079601574 |
| chr17.0073852400 | chr5.0016941153 | chr15.0068872137 |
| chr2.0055747731 | chr14.0065717002 | chr22.0047242843 |
| chr12.0001675739 | chr16.0022529397 | chr20.0044458275 |
| chr17.0053343752 | chr3.0052502709 | chr19.0001367607 |
| chr15.0067388306 | chr20.0035113039 | chr12.0091564554 |
| chr20.0025033000 | chr11.0000370878 | chr5.0176295797 |
| chr18.0055733104 | chr6.0150097548 | chr16.0058824222 |
| chr14.0091225499 | chr16.0000812359 | chr13.0111766235 |
| chrX.0048648229 | chr10.0047627474 | chr3.0012923687 |
| chr19.0050919627 | chr12.0132682730 | chr16.0004604746 |
| chr3.0101280900 | chr18.0040176376 | chr15.0101866025 |
| chr20.0061797025 | chr12.0117382383 | chr11.0065358078 |
| chr7.0055538445 | chr13.0046244319 | chr22.0025600824 |
| chr5.0180670930 | chr6.0013797973 | chr15.0068583382 |
| chr12.0014050295 | chr13.0110882278 | chr13.0034225558 |
| chr20.0036544069 | chr18.0034958835 | chr16.0058757515 |
| chr6.0015597103 | chr5.0015575251 | chr3.0120314864 |
| chr12.0022883124 | chr15.0030119387 | chr15.0025955428 |
| chr16.0050583740 | chr14.0069403414 | chr15.0041080358 |
| chr20.0061089342 | chr16.0058115584 | chr6.0151404627 |
| chr11.0027181331 | chr15.0045409777 | chr15.0051203970 |
| chr15.0066910189 | chr5.0000320856 | chr15.0091398741 |
| chr10.0102985949 | chr14.0074179572 | chr22.0019865215 |
| chr12.0020575002 | chr20.0044681604 | chr12.0029430585 |
| chr6.0030102506 | chr6.0111791900 | chr17.0073633572 |
| chr11.0068848057 | chr9.0125487085 | chr12.0058003396 |
| chr6.0084881773 | chr15.0043425672 | chr20.0035325831 |
| chr3.0197183177 | chr2.0045157529 | chr6.0027780652 |
| chr3.0184456165 | chr6.0073974640 | chr19.0004099560 |
| chr9.0140099440 | chr19.0038987939 | chr18.0056046635 |
| chr18.0047448008 | chr16.0031439662 | chr5.0035853643 |
| chr5.0070680746 | chr15.0059597553 | chr15.0064816687 |
| chr3.0056400417 | chr6.0013302952 | chrX.0049178276 |
| chr12.0122062866 | chr22.0032845764 | chrX.0006976158 |
| chr18.0006408773 | chr11.0057282706 | chr14.0020835244 |
| chr12.0129279448 | chr3.0132072978 | chr15.0079071423 |
| chr17.0010520145 | chr18.0008744244 | chr2.0226496814 |
| chr13.0114005848 | chr12.0054389997 | chr17.0077021668 |
| chr5.0080598012 | chr3.0172428265 | chr15.0082273478 |
| chr5.0000513838 | chr12.0123568623 | chr12.0120832604 |
| chr14.0063478499 | chr22.0030086226 | chr3.0182830735 |
| chr22.0030900619 | chr9.0140286118 | chr2.0033057636 |
| chr12.0007052710 | chr12.0122517879 | chr13.0099129570 |
| chr3.0035706161 | chr3.0056929786 | chr12.0008857266 |
| chrX.0049643284 | chr6.0004074381 | chr19.0046196188 |
| chr11.0071458506 | chr11.0061098423 | chr11.0114042360 |
| chr20.0041366127 | chr12.0097384251 | chr14.0031513408 |
| chr12.0003602291 | chr15.0026115000 | chr6.0036799126 |
| chr15.0094260862 | chr6.0034051016 | chr15.0067062701 |
| chr12.0086624315 | chr20.0049196640 | chr17.0042857648 |
| chr15.0076639476 | chr22.0031738796 | chr14.0024778911 |
| chr19.0056831634 | chr10.0135478018 | chr15.0084324496 |
| chr7.0006629867 | chr5.0000841665 | chr17.0017415526 |
| chr2.0192015446 | chr14.0065488123 | chr15.0064227585 |
| chr16.0075130142 | chr20.0001929715 | chr18.0046834838 |
| chr16.0004712237 | chr6.0000198033 | chr3.0151630654 |
| chr3.0197326166 | chr15.0029211186 | chr11.0061341207 |
| chr8.0080733486 | chr2.0176032527 | chr3.0108046904 |
| chr11.0070953873 | chr7.0102135905 | chr15.0071754544 |
| chr16.0028884493 | chr9.0135116332 | chr20.0058035543 |
| chr20.0044819126 | chr2.0202507780 | chr12.0049603111 |
| chr20.0000823025 | chr22.0030106707 | chr18.0072175316 |
| chr3.0057193675 | chr5.0177715583 | chr12.0001087688 |
| chr22.0045980075 | chr18.0012883118 | chr5.0178715673 |
| chr22.0031740789 | chr7.0029235015 | chr13.0051396659 |
| chr20.0057417557 | chr14.0065758678 | chr22.0033886653 |
| chr13.0095260109 | chr14.0095999996 | chr5.0007824030 |
| chr11.0128362132 | chr13.0099226141 | chr22.0025284817 |
| chr15.0062359382 | chr15.0090563725 | chr11.0123445488 |
| chr14.0019500361 | chr12.0081330728 | chr14.0091022261 |
| chr12.0062585750 | chr17.0000908318 | chr2.0098487920 |
| chr5.0069204638 | chr22.0033106982 | chr22.0039967081 |
| chr12.0053460222 | chr15.0020800162 | chr2.0099422979 |
| chr6.0037600239 | chr12.0001099231 | chr13.0113746487 |
| chr20.0032784518 | chr6.0018262136 | chrX.0049359038 |
| chr18.0007116462 | chr2.0110702857 | chr11.0011613810 |
| chr2.0237109274 | chr13.0028497262 | chr2.0219124247 |
| chr12.0049955505 | chr14.0038093987 | chr12.0092402155 |
| chr11.0041137301 | chr22.0036866546 | chr11.0002158119 |
| chr17.0079375808 | chr2.0204105168 | chr19.0052992415 |
| chr3.0127634119 | chr5.0174184394 | chr2.0116280845 |
| chr9.0130266415 | chr11.0111316435 | chr9.0038621990 |
| chr2.0130806301 | chr12.0124344718 | chr18.0045862942 |
| chr18.0003604915 | chr12.0118500570 | chr13.0115001386 |
| chr19.0048673525 | chr12.0114122054 | chr12.0000662536 |
| chr3.0146321247 | chr22.0021570530 | chr8.0144853018 |
| chr3.0013894328 | chr11.0122753928 | chr14.0105048321 |
| chr18.0045723996 | chr12.0054424639 | chr13.0042614083 |
| chr15.0056544942 | chr12.0106530922 | chr16.0000583839 |
| chr16.0028374665 | chr16.0011597648 | chr3.0037333531 |
| chr6.0162311892 | chr9.0125512943 | chr13.0028204300 |
| chr16.0070834848 | chr17.0017848335 | chr15.0088721801 |
| chr20.0043513977 | chr19.0010207350 | chr15.0080624605 |
| chr20.0009596603 | chr14.0032395968 | chr16.0078140608 |
| chr20.0043213955 | chr22.0045710340 | chr12.0023350005 |
| chr2.0066809303 | chr3.0077420946 | chr9.0075217586 |
| chr15.0075986380 | chr7.0156741855 | chr6.0046368470 |
| chr22.0018661095 | chr14.0105185236 | chr11.0071150631 |
| chr18.0077166371 | chr15.0032083799 | chr4.0112728255 |
| chr22.0031886188 | chr14.0103561766 | chr14.0096552963 |
| chr6.0028574981 | chr18.0012419897 | chr15.0093906869 |
| chr15.0058725966 | chr20.0002675487 | chr10.0108520582 |
| chr20.0050079494 | chr12.0108525950 | chr11.0031121358 |
| chr5.0090575842 | chr8.0141249150 | chr11.0003440420 |
| chr12.0063277133 | chr15.0028878400 | chr3.0027929607 |
| chr6.0044223188 | chr20.0025839615 | chr9.0092333879 |
| chr7.0005324731 | chr16.0070380240 | chr15.0084912101 |
| chr10.0126183240 | chr17.0078725830 | chr12.0110438723 |
| chr20.0048064230 | chr2.0074120215 | chr19.0033896553 |
| chr18.0056941246 | chr17.0078235740 | chr12.0024898838 |
| chr4.0183063459 | chr12.0125810688 | chr10.0065475234 |
| chr22.0048973497 | chr15.0080503496 | chr2.0023908317 |
| chr20.0000470768 | chr6.0035049824 | chr13.0021286449 |
| chr6.0021847663 | chr19.0002675391 | chr22.0039006789 |
| chr14.0105525484 | chr20.0037356960 | chr3.0007060708 |
| chr13.0113528544 | chr18.0025730190 | chr6.0075912320 |
| chr6.0167188753 | chr17.0036349155 | chr12.0115161241 |
| chr11.0021317728 | chr13.0022010106 | chr13.0094140396 |
| chr12.0012364177 | chr9.0133912465 | chr14.0024785859 |
| chr12.0119344045 | chr16.0011595555 | chr8.0133070210 |
| chr8.0144896409 | chr13.0048891926 | chr22.0050508487 |
| chr2.0231913195 | chr2.0025097119 | chr18.0071611180 |
| chr17.0081046973 | chr17.0019770122 | chr20.0015588271 |
| chr4.0007652024 | chr7.0021744160 | chr5.0039219698 |
| chr11.0006341282 | chr8.0122652473 | chr20.0042887008 |
| chr14.0100151872 | chr5.0115889873 | chr18.0045057574 |
| chr22.0033384015 | chr2.0035092870 | chr17.0034501886 |
| chr12.0103534118 | chr2.0204118963 | chr16.0079166247 |
| chr20.0031151987 | chr11.0000773106 | chr4.0112764777 |
| chr11.0065744445 | chr14.0023845293 | chr3.0018656752 |
| chr6.0003356742 | chr18.0004254337 | chr16.0079250798 |
| chr12.0016086133 | chr18.0059728930 | chr14.0075565952 |
| chr20.0009049993 | chr15.0039873517 | chr12.0113690924 |
| chr10.0026728514 | chr16.0023516312 | chr11.0132475539 |
| chr22.0037055690 | chr2.0213871002 | chr13.0096687076 |
| chr19.0004019341 | chr17.0036357651 | chr12.0088429374 |
| chr3.0185977002 | chr18.0005797710 | chr17.0062777136 |
| chr18.0046444366 | chr14.0056068749 | chr3.0141163289 |
| chr8.0011415272 | chr20.0030158713 | chr22.0024547472 |
| chr11.0095823245 | chr9.0127231878 | chr20.0024958641 |
| chr12.0113902624 | chr11.0000407417 | chr11.0120447543 |
| chr22.0046895712 | chr19.0021542535 | chr16.0089778878 |
| chr18.0059948461 | chr12.0050899260 | chr14.0074791015 |
| chr6.0116637077 | chr9.0073216229 | chr5.0019575004 |
| chr3.0079476146 | chr11.0131764597 | chr22.0040358235 |
| chr12.0000248545 | chr10.0050114638 | chr16.0070207297 |
| chr13.0070357073 | chr14.0073112691 | chr18.0045686939 |
| chr10.0116528581 | chr14.0024294681 | chr6.0161063597 |
| chr15.0102042815 | chr18.0042261338 | chr13.0026538260 |
| chr11.0064513156 | chr6.0017722030 | chr10.0068355194 |
| chr6.0035052726 | chr12.0122525036 | chr17.0019035897 |
| chr18.0077282967 | chr12.0095466691 | chr15.0066311980 |
| chr3.0159783251 | chr14.0074978044 | chr11.0066315845 |
| chr3.0021993057 | chr19.0046993480 | chr12.0057856280 |
| chr12.0108001160 | chr18.0011999639 | chr12.0033048797 |
| chr10.0073345500 | chr16.0070209492 | chr6.0151652742 |
| chr18.0021084095 | chr12.0016360748 | chr12.0005562121 |
| chr3.0145878214 | chr6.0162376364 | chr6.0003438536 |
| chr20.0001908104 | chr20.0010561357 | chr19.0006913486 |
| chr16.0083967135 | chr3.0197517516 | chr17.0040439892 |
| chr11.0035683582 | chr9.0139089308 | chr14.0023070923 |
| chr7.0103971806 | chr5.0033935968 | chr2.0233983675 |
| chr2.0000294658 | chr15.0029078025 | chr22.0042606263 |
| chr12.0096350795 | chr11.0115228019 | chr15.0100510425 |
| chr2.0240197131 | chr7.0064030245 | chr12.0110224997 |
| chr20.0023333978 | chr16.0004987613 | chr13.0053624408 |
| chr13.0046040265 | chr11.0002473803 | chr15.0085245022 |
| chr13.0102109190 | chr14.0080132110 | chr16.0011595555 |
| chr14.0100008656 | chr22.0042375785 | chr12.0027574674 |
| chr19.0037288810 | chr14.0020295845 | chr12.0131691177 |
| chr13.0113086352 | chr18.0039617068 | chr12.0113684022 |
| chr16.0008892909 | chr5.0140226052 | chr16.0084857412 |
| chr22.0036769509 | chr12.0133143645 | chr12.0024994558 |
| chr16.0087637417 | chr16.0030797933 | chr10.0117820694 |
| chr3.0184304408 | chr17.0003858591 | chr3.0018656789 |
| chr16.0054316968 | chr6.0161065266 | chr2.0222301426 |
| chr5.0156675967 | chr6.0154624519 | chr20.0062257281 |
| chr2.0241636085 | chr20.0060281200 | chr16.0002478275 |
| chr18.0061160438 | chr17.0037310064 | chr7.0098246890 |
| chr6.0125125685 | chr6.0015525504 | chr18.0074151887 |
| chr20.0053220184 | chr9.0042036639 | chr2.0103747925 |
| chr18.0009135991 | chr17.0028994426 | chr12.0085672242 |
| chr13.0070681911 | chr16.0087661398 | chr19.0000751159 |
| chr7.0002738546 | chr14.0105394744 | chr8.0145046909 |
| chr2.0005749552 | chr18.0077209053 | chr19.0000608811 |
| chr13.0111837470 | chr12.0056649722 | chr6.0053524907 |
| chr6.0013153213 | chr13.0078283953 | chr15.0080588199 |
| chr12.0006981014 | chr16.0029790927 | chr15.0079233994 |
| chr20.0058634065 | chr15.0074891625 | chr12.0129408895 |
| chr8.0022551030 | chr15.0077881379 | chr12.0025937825 |
| chr13.0053205778 | chr11.0117582399 | chr18.0072250823 |
| chr16.0088637645 | chr6.0150184172 | chr16.0058760779 |
| chr13.0095890714 | chr18.0074815924 | chr11.0125773076 |
| chr11.0118230644 | chr16.0030441289 | chr16.0085744228 |
| chr14.0037046398 | chr6.0010421442 | chr6.0116464670 |
| chr22.0044392082 | chr12.0067284004 | chr6.0022021274 |
| chr7.0120969027 | chr19.0050193737 | chr2.0227661381 |
| chr18.0030650346 | chr14.0101539776 | chr14.0075033873 |
| chr14.0034931900 | chr20.0031352450 | chr17.0015380552 |
| chr3.0071113322 | chr13.0035146074 | chr15.0101536131 |
| chr14.0068830387 | chr11.0070484675 | chr16.0083174768 |
| chr2.0135160156 | chr12.0100593108 | chr20.0007889631 |
| chr22.0035724388 | chr11.0007034933 | chr15.0065563447 |
| chr14.0093076860 | chr20.0016734749 | chr20.0037174617 |
| chr14.0019850191 | chr5.0178715673 | chr5.0179114073 |
| chr7.0005535020 | chr16.0010635319 | chr12.0018568899 |
| chr17.0018941971 | chr9.0130867610 | chr12.0049409447 |
| chr3.0110697945 | chr14.0020111288 | chr3.0119813692 |
| chr13.0074637138 | chr19.0054413591 | chr3.0151160316 |
| chr22.0017849502 | chrX.0101726635 | chr9.0112810447 |
| chr5.0095710534 | chr17.0018865314 | chr2.0039419989 |
| chr22.0037466349 | chr20.0049196788 | chr17.0012769886 |
| chr17.0062812567 | chr15.0069756183 | chr14.0105034743 |
| chr3.0138918148 | chr11.0044962782 | chr15.0052550022 |
| chr11.0064708471 | chr18.0077218474 | chr11.0061310165 |
| chr11.0047437823 | chr6.0161034075 | chr14.0092901413 |
| chr13.0096206771 | chr12.0122245069 | chr11.0062357965 |
| chr16.0061703704 | chr14.0064832649 | chr11.0134214876 |
| chr11.0061515776 | chr12.0072082304 | chr3.0025114359 |
| chr14.0073202793 | chr20.0062609849 | chr3.0046503548 |
| chr15.0044961718 | chr14.0100488657 | chr13.0026590625 |
| chr11.0117944246 | chr19.0049118462 | chr5.0172106425 |
| chr22.0037536084 | chr22.0018804154 | chr18.0010485679 |
| chr11.0130320107 | chr12.0075879423 | chr17.0012479544 |
| chr3.0049029310 | chr16.0016460947 | chr16.0068174112 |
| chr19.0013051211 | chr19.0004558437 | chr7.0100812136 |
| chr6.0158988252 | chr20.0036661494 | chr19.0048669578 |
| chr13.0039336867 | chr18.0034958684 | chr14.0073686279 |
| chr10.0070939275 | chr12.0129098203 | chr8.0133928368 |
| chr20.0062066022 | chr18.0002878772 | chr10.0098457342 |
| chr14.0105032414 | chr11.0068611806 | chr11.0043605047 |
| chr3.0124530718 | chr15.0029075704 | chr3.0055518374 |
| chr13.0088329151 | chr16.0066505404 | chr6.0032038784 |
| chr12.0113313505 | chr12.0006638758 | chr17.0015394553 |
| chr7.0016685981 | chr16.0087890498 | chr10.0135088481 |
| chr15.0078406033 | chr18.0032113260 | chr10.0028726670 |
| chr12.0133532241 | chr17.0078293742 | chr16.0029187478 |
| chr8.0145017295 | chr15.0091422745 | chr18.0013140322 |
| chr16.0017322561 | chr15.0045249864 | chr18.0057193836 |
| chr13.0099196034 | chr18.0034852944 | chr15.0088583056 |
| chr13.0103047839 | chr14.0105678272 | chr10.0073020119 |
| chr11.0031841691 | chr15.0074485214 | chr17.0007260381 |
| chr9.0006414308 | chr5.0176761538 | chr2.0052278383 |
| chr17.0065821666 | chr16.0024550666 | chr15.0068126938 |
| chr6.0169041074 | chr12.0057617584 | chr12.0003126663 |
| chr2.0000290336 | chr6.0082379185 | chr19.0039622344 |
| chr6.0166749267 | chr18.0061642608 | chr18.0020959985 |
| chr14.0023566213 | chr3.0140795796 | chr14.0060976611 |
| chr17.0015686374 | chr3.0030789979 | chr11.0074247056 |
| chr20.0058629826 | chr12.0083273710 | chr9.0130395772 |
| chr3.0130999612 | chr3.0123150850 | chr9.0140239835 |
| chr17.0019013773 | chr17.0039824390 | chr16.0058789143 |
| chr2.0192876773 | chr10.0080833721 | chr9.0129726622 |
| chr20.0004925421 | chr20.0013956941 | chr12.0004810242 |
| chr14.0058124290 | chr8.0145744056 | chr15.0069794562 |
| chr20.0030679997 | chr16.0070723448 | chr3.0195616762 |
| chr22.0029197031 | chr12.0065337511 | chr9.0079448403 |
| chr6.0075995899 | chr18.0011747918 | chr22.0038507208 |
| chr3.0109366536 | chr6.0031091704 | chr3.0174382932 |
| chr20.0031053633 | chr12.0127526344 | chr22.0016645749 |
| chr17.0043238278 | chr11.0117630311 | chr3.0046490317 |
| chr15.0034784699 | chr11.0035968781 | chr6.0151915879 |
| chr3.0197678316 | chr11.0065154531 | chr12.0062584454 |
| chr12.0086512077 | chr20.0016415868 | chr9.0140352405 |
| chr14.0021544397 | chr18.0001012129 | chr3.0020088712 |
| chr3.0048647168 | chr18.0019925911 | chr20.0003661537 |
| chr5.0058633250 | chr13.0033048801 | chr12.0130962583 |
| chr17.0046125527 | chr6.0043631088 | chr2.0240905174 |
| chr14.0072621904 | chr20.0022567986 | chr18.0046385823 |
| chr16.0055478577 | chr3.0016397888 | chr12.0099615042 |
| chr20.0021685842 | chr20.0021093596 | chr15.0050280046 |
| chr15.0033278522 | chr18.0013912474 | chr18.0044478678 |
| chr7.0066594276 | chr16.0000835648 | chr20.0062153435 |
| chr18.0029340825 | chr18.0008501443 | chr22.0050735280 |
| chr17.0047472491 | chr17.0044895852 | chr14.0064598744 |
| chr3.0171788357 | chr6.0038148615 | chr12.0125017105 |
| chr22.0021796464 | chr5.0080491458 | chr19.0007069967 |
| chr14.0078228378 | chr11.0131392084 | chr12.0101787956 |
| chr18.0046462840 | chr11.0118393450 | chr16.0002091062 |
| chr16.0023653028 | chr7.0004307133 | chr18.0008264490 |
| chr6.0128353091 | chr14.0104696055 | chr18.0019067987 |
| chr20.0031052159 | chr6.0073356528 | chr8.0010191232 |
| chr12.0027821943 | chr17.0006375439 | chr17.0055520767 |
| chr11.0064708849 | chr14.0088458693 | chr15.0051604337 |
| chr6.0151647119 | chr3.0184209533 | chr20.0061428843 |
| chr8.0097166862 | chr9.0112016299 | chr12.0131689812 |
| chr20.0037053748 | chr12.0132836081 | chr3.0060919598 |
| chr12.0100967375 | chr11.0061532277 | chr6.0064254064 |
| chr3.0048554238 | chr5.0140202715 | chr20.0045845539 |
| chr5.0006453988 | chr11.0012983337 | chr6.0090109650 |
| chr20.0019426606 | chr15.0072581958 | chr6.0146505684 |
| chr17.0046707012 | chr2.0059054529 | chr13.0028550394 |
| chr14.0031419741 | chr17.0034802392 | chr15.0069952346 |
| chr12.0058166267 | chr14.0023901954 | chr20.0062134308 |
| chr16.0067552788 | chr22.0032358489 | chr7.0129996892 |
| chr6.0144675770 | chr16.0087274304 | chr14.0039262070 |
| chr15.0092399584 | chr6.0042897901 | chr2.0216879665 |
| chr11.0126398134 | chr3.0123440328 | chr3.0153560971 |
| chr6.0168842694 | chr3.0124369676 | chr18.0025139835 |
| chr3.0053189890 | chr13.0093114991 | chr20.0006101929 |
| chr16.0089116180 | chr18.0055978637 | chr5.0011248947 |
| chr22.0040709984 | chr20.0060141507 | chr14.0100142498 |
| chr3.0183981132 | chr12.0117176320 | chr3.0017562733 |
| chr3.0197676776 | chr14.0101123919 | chr13.0114802848 |
| chr6.0167656016 | chr12.0041671324 | chr2.0024165918 |
| chr3.0048935648 | chr16.0003148824 | chr12.0111902693 |
| chr19.0054407970 | chr15.0101877370 | chr13.0041773854 |
| chr20.0061143247 | chr6.0157855779 | chr14.0077681144 |
| chr16.0050318815 | chr15.0060904614 | chr12.0001400446 |
| chr11.0124767781 | chr20.0034464349 | chr13.0057743067 |
| chr12.0058158604 | chr10.0124361495 | chr20.0019192507 |
| chr7.0105621308 | chr12.0131170616 | chr12.0053678199 |
| chr13.0028499179 | chr11.0118788893 | chr5.0053421823 |
| chr20.0034982498 | chr13.0020711105 | chr12.0066049238 |
| chr3.0147107783 | chr16.0018487990 | chr11.0066364146 |
| chr3.0051741520 | chr2.0024308827 | chr3.0123550508 |
| chr3.0085099538 | chr5.0088124219 | chr3.0068791660 |
| chr13.0028431960 | chr9.0075217586 | chr17.0046221813 |
| chr11.0107660794 | chr20.0022548751 | chr5.0022752463 |
| chr22.0043321825 | chr13.0048978197 | chr16.0069662747 |
| chr16.0081482070 | chr12.0108955777 | chr6.0026856434 |
| chr6.0029531351 | chr22.0049100588 | chr14.0089721594 |
| chr3.0156193747 | chr22.0042912017 | chr14.0104178670 |
| chr20.0014983291 | chr8.0143881060 | chr12.0003241735 |
| chr17.0019028901 | chr22.0024169281 | chr17.0043356139 |
| chr6.0138825229 | chr5.0079789558 | chr7.0147581299 |
| chr12.0116987790 | chr5.0073668732 | chr16.0083606272 |
| chr17.0034059388 | chr15.0031862272 | chr9.0139651444 |
| chr15.0094015466 | chr8.0003254696 | chr18.0056146980 |
| chr17.0039968547 | chr18.0011995660 | chr6.0102097023 |
| chr3.0032570450 | chr9.0129263152 | chr19.0035611941 |
| chr5.0112629019 | chr16.0015006658 | chr15.0057403882 |
| chr20.0062786564 | chr2.0152855523 | chr15.0039802950 |
| chr8.0041655751 | chr8.0143292052 | chr11.0003144685 |
| chr16.0068569850 | chr6.0130173399 | chr17.0016182623 |
| chr10.0023633690 | chr20.0060724089 | chr8.0086556290 |
| chr22.0043442768 | chr18.0074914519 | chr16.0087698990 |
| chr3.0128201657 | chr9.0138848831 | chr15.0044284871 |
| chr9.0132022062 | chr2.0134023445 | chr13.0051846552 |
| chr17.0034493838 | chr15.0043976544 | chr17.0006001954 |
| chr6.0058181382 | chr2.0211527868 | chr14.0100291764 |
| chr2.0110012891 | chr17.0040260704 | chr15.0073631717 |
| chr9.0095650688 | chr19.0010765660 | chr3.0047116517 |
| chr14.0088177556 | chr13.0101203394 | chr16.0027852176 |
| chr14.0038069872 | chr6.0038176362 | chr11.0111316435 |
| chr3.0197387682 | chr2.0235936653 | chr17.0020909292 |
| chr11.0069831442 | chr20.0062150117 | chr12.0123135330 |
| chr17.0002241533 | chr17.0013849430 | chr14.0023201418 |
| chr15.0032394508 | chr11.0078920341 | chr3.0014041701 |
| chr16.0021546728 | chr19.0019248569 | chr18.0056064648 |
| chr3.0195628069 | chr6.0157892958 | chr9.0134184484 |
| chr14.0078227825 | chr18.0077629407 | chr18.0012466006 |
| chr8.0001250451 | chr2.0010092655 | chr10.0080956875 |
| chr14.0101722707 | chr17.0015902161 | chr14.0077523673 |
| chr6.0041236624 | chr5.0140735246 | chr6.0034463322 |
| chr20.0061146919 | chr8.0066955958 | chr14.0071251384 |
| chr3.0167966552 | chr17.0017881005 | chr20.0031129482 |
| chr13.0108411649 | chr13.0110920747 | chr16.0058790064 |
| chr5.0087975812 | chr6.0161066903 | chr20.0060061483 |
| chr20.0003761969 | chr11.0068925167 | chr9.0134177501 |
| chr20.0031124002 | chr9.0133327442 | chr13.0036104053 |
| chr2.0105472738 | chr1.0228744889 | chr19.0013136364 |
| chr3.0013729876 | chr5.0145583807 | chr16.0010906798 |
| chr18.0043956019 | chr22.0021049955 | chr3.0126713940 |
| chr12.0021591368 | chr9.0130291176 | chr15.0091398642 |
| chr11.0124400388 | chr14.0101016626 | chr8.0067351838 |
| chr3.0182818648 | chr3.0193775849 | chr18.0040084697 |
| chr6.0029690766 | chr20.0058840198 | chr18.0003580666 |
| chr19.0046992651 | chr20.0058421070 | chr11.0020039824 |
| chr20.0002539227 | chr2.0240145464 | chr18.0056046953 |
| chr13.0024839413 | chr11.0069259330 | chr12.0083248478 |
| chr12.0003145799 | chr17.0031836661 | chr18.0034431276 |
| chr5.0016593335 | chr16.0033108204 | chr12.0006650910 |
| chr12.0133100772 | chr18.0010740861 | chr17.0043631042 |
| chr12.0096429846 | chr14.0075535293 | chr11.0132717823 |
| chr3.0134203994 | chr6.0080816434 | chr20.0033566149 |
| chr18.0072120483 | chr18.0019559123 | chr3.0043677568 |
| chr18.0069578038 | chr17.0015690046 | chr2.0102623275 |
| chr15.0063746873 | chr6.0032937819 | chr17.0027056417 |
| chr13.0019579068 | chr17.0003416652 | chr13.0042769050 |
| chr3.0047314564 | chr15.0093552495 | chr12.0123753032 |
| chr13.0031383575 | chr15.0050720608 | chr20.0055066923 |
| chr20.0017949564 | chr8.0001595915 | chr11.0001245363 |
| chr16.0046940916 | chr9.0024542255 | chr12.0120996668 |
| chr5.0050674729 | chr14.0019948447 | chr18.0046102929 |
| chr11.0064761978 | chr17.0015546785 | chr6.0033643123 |
| chr3.0021666073 | chrX.0153167693 | chr20.0006759498 |
| chr8.0052796882 | chr2.0063278108 | chr12.0001312523 |
| chr2.0233351810 | chr20.0056185671 | chr7.0143885174 |
| chr3.0125692978 | chr18.0034034835 | chr16.0029296720 |
| chr15.0023301221 | chr9.0112760119 | chr5.0010571406 |
| chr11.0003945589 | chr19.0049932161 | chr12.0067087331 |
| chr14.0099984633 | chr15.0087274357 | chr17.0036059462 |
| chr19.0000847361 | chr5.0164550214 | chr12.0001414241 |
| chr11.0120622887 | chr5.0139488638 | chr17.0020940382 |
| chr2.0232393103 | chr22.0031255145 | chr13.0026508624 |
| chr20.0044987164 | chr16.0018945440 | chr17.0016744139 |
| chr11.0000309045 | chr9.0138603854 | chr9.0085110033 |
| chr16.0048468530 | chr15.0062542489 | chr18.0029303612 |
| chr11.0018814074 | chr3.0005227732 | chr12.0054366343 |

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of detecting cerebral palsy in a subject, the method comprising:
determining a methylation state for each of a plurality of cytosine-guanine dinucleotide (CpG) sites in a sample obtained from the subject,
calculating a differential methylation level based on the methylation states of the plurality of CpG sites, and
comparing the differential methylation level to a predetermined reference level, wherein when the differential methylation level deviates from the predetermined reference level, the presence of cerebral palsy is indicated in the patient.

2. The method of claim 1 wherein the plurality of CpG sites comprises at least one selected from the CpG sites listed in Table 4, Table 5, Table 6 or Table 7.

3. The method of claim 1 wherein the plurality of CpG sites comprises at least five selected from the CpG sites listed in Table 4, Table 5, Table 6 or Table 7.

4. The method of claim 1 wherein the plurality of CpG sites comprises at least ten selected from the CpG sites listed in Table 4, Table 5, Table 6 or Table 7.

5. The method of claim 1 wherein the plurality of CpG sites comprises at least m % selected from the top n most predictive CpG sites listed in Table 4 or Table 5, wherein:
m is selected from the group consisting of: 50, 60, 70, 80, 90, 95, and 99; and
n is selected from the group consisting of 100, 500, 1,000, 2,000, and 2,500.

6. The method of claim 1, further comprising:
providing treatment for cerebral palsy to the patient when cerebral palsy is indicated.

7. The method of claim 6 wherein treatment for cerebral palsy comprises the administration of medication, therapy or surgery.

8. The method of claim 1 wherein the plurality of CpG sites comprises:
a plurality of up sites, and
a plurality of down sites,
wherein:
methylation at the up sites indicates that the likelihood the patient has cerebral palsy is increased, and
methylation at the down sites indicates that the likelihood the patient has cerebral palsy is decreased, and
wherein calculating a differential methylation level comprises adding in a linear weighted summation values based on the methylation states of the up sites from a value based on the methylation states of the down sites.

9. The method of claim 1, wherein the sample is selected from the group consisting of a peripheral blood mononuclear cell sample, a blood sample, a muscle sample or a satellite cell sample.
